SEQUENCE LISTING (62 sequences)

SEQ ID NO: 1 - protein, 4 aa, Artificial Sequence
  Synthetic
LIGK

SEQ ID NO: 2 - protein, 5 aa, Artificial Sequence
  Synthetic
LIGKV

SEQ ID NO: 3 - protein, 4 aa, Artificial Sequence
  Synthetic
KGIL

SEQ ID NO: 4 - protein, 3 aa, Artificial Sequence
  Synthetic
KGI

SEQ ID NO: 5 - protein, 3 aa, Artificial Sequence
  Synthetic
AGI

SEQ ID NO: 6 - protein, 3 aa, Artificial Sequence
  Synthetic
IGA

SEQ ID NO: 7 - protein, 3 aa, Artificial Sequence
  Synthetic
KGA

SEQ ID NO: 8 - protein, 3 aa, Artificial Sequence
  Synthetic
KGA

SEQ ID NO: 9 - protein, 3 aa, Artificial Sequence
  Synthetic
KAI

SEQ ID NO: 10 - protein, 3 aa, Artificial Sequence
  Synthetic
IAK

SEQ ID NO: 11 - protein, 3 aa, Artificial Sequence
  Synthetic
RGI

SEQ ID NO: 12 - protein, 3 aa, Artificial Sequence
  Synthetic
IGR

SEQ ID NO: 13 - protein, 3 aa, Artificial Sequence
  Synthetic
XGI

SEQ ID NO: 14 - protein, 3 aa, Artificial Sequence
  Synthetic
XGI

SEQ ID NO: 15 - protein, 3 aa, Artificial Sequence
  Synthetic
IGX

SEQ ID NO: 16 - protein, 3 aa, Artificial Sequence
  Synthetic
IGX

SEQ ID NO: 17 - protein, 4 aa, Artificial Sequence
  Synthetic
LIGX

SEQ ID NO: 18 - protein, 4 aa, Artificial Sequence
  Synthetic
XGIL

SEQ ID NO: 19 - protein, 4 aa, Artificial Sequence
  Synthetic
LIGX

SEQ ID NO: 20 - protein, 4 aa, Artificial Sequence
  Synthetic
XGIL

SEQ ID NO: 21 - protein, 4 aa, Artificial Sequence
  Synthetic
LIGX

SEQ ID NO: 22 - protein, 4 aa, Artificial Sequence
  Synthetic
XGIL

SEQ ID NO: 23 - protein, 3 aa, Artificial Sequence
  Synthetic
XGI

SEQ ID NO: 24 - protein, 3 aa, Artificial Sequence
  Synthetic
IGX

SEQ ID NO: 25 - protein, 4 aa, Artificial Sequence
  Synthetic
LIGX

SEQ ID NO: 26 - protein, 4 aa, Artificial Sequence
  Synthetic
LIGX

SEQ ID NO: 27 - protein, 4 aa, Artificial Sequence
  Synthetic
XXXX

SEQ ID NO: 28 - protein, 3 aa, Artificial Sequence
  Synthetic
XXX

SEQ ID NO: 29 - protein, 4 aa, Artificial Sequence
  Synthetic
LIGX

SEQ ID NO: 30 - protein, 3 aa, Artificial Sequence
  Synthetic
IGX

SEQ ID NO: 31 - protein, 3 aa, Artificial Sequence
  Synthetic
IGX

SEQ ID NO: 32 - protein, 4 aa, Artificial Sequence
  Synthetic
LIGR

SEQ ID NO: 33 - protein, 4 aa, Artificial Sequence
  Synthetic
LIGD

SEQ ID NO: 34 - protein, 4 aa, Artificial Sequence
  Synthetic
LIGE

SEQ ID NO: 35 - protein, 4 aa, Artificial Sequence
  Synthetic
LIGN

SEQ ID NO: 36 - protein, 4 aa, Artificial Sequence
  Synthetic
LIGQ

SEQ ID NO: 37 - protein, 4 aa, Artificial Sequence
  Synthetic
LIGS

SEQ ID NO: 38 - protein, 4 aa, Artificial Sequence
  Synthetic
LIGT

SEQ ID NO: 39 - protein, 4 aa, Artificial Sequence
  Synthetic
LIGY

SEQ ID NO: 40 - protein, 4 aa, Artificial Sequence
  Synthetic
LIPK

SEQ ID NO: 41 - protein, 4 aa, Artificial Sequence
  Synthetic
LPGK

SEQ ID NO: 42 - protein, 4 aa, Artificial Sequence
  Synthetic
LIGH

SEQ ID NO: 43 - protein, 3 aa, Artificial Sequence
  Synthetic
LXK

SEQ ID NO: 44 - protein, 4 aa, Artificial Sequence
  Synthetic
LXGK

SEQ ID NO: 45 - protein, 3 aa, Artificial Sequence
  Synthetic
LXK

SEQ ID NO: 46 - protein, 4 aa, Artificial Sequence
  Synthetic
LXGK

SEQ ID NO: 47 - protein, 3 aa, Artificial Sequence
  Synthetic
LXK

SEQ ID NO: 48 - protein, 4 aa, Artificial Sequence
  Synthetic
LXGK

SEQ ID NO: 49 - protein, 3 aa, Artificial Sequence
  Synthetic
LXK

SEQ ID NO: 50 - protein, 4 aa, Artificial Sequence
  Synthetic
LXGK

SEQ ID NO: 51 - protein, 4 aa, Artificial Sequence
  Synthetic
LIGM

SEQ ID NO: 52 - protein, 6 aa, Artificial Sequence
  Synthetic
SLIGKV

SEQ ID NO: 53 - protein, 6 aa, Murinae sp.
  MOD_RES (6)..(6): AMIDATION
SLIGRL

SEQ ID NO: 54 - protein, 6 aa, Artificial Sequence
  Synthetic
SLIAKV

SEQ ID NO: 55 - protein, 6 aa, Artificial Sequence
  Synthetic
SLIGKA

SEQ ID NO: 56 - protein, 6 aa, Artificial Sequence
  Synthetic
SFLLRN

SEQ ID NO: 57 - protein, 5 aa, Artificial Sequence
  Synthetic
SLIGK

SEQ ID NO: 58 - protein, 6 aa, Artificial Sequence
  Synthetic
ALIGKV

SEQ ID NO: 59 - protein, 6 aa, Artificial Sequence
  Synthetic
SAIGKV

SEQ ID NO: 60 - protein, 6 aa, Artificial Sequence
  Synthetic
SLAGKV

SEQ ID NO: 61 - protein, 6 aa, Artificial Sequence
  Synthetic
SLIGAV

SEQ ID NO: 62 - protein, 4 aa, Artificial Sequence
  Synthetic
LIGK

(19) United States
(12) Patent Application Publication
(10) Pub. No.: US 2006/0063930 A1
(43) Pub. Date: Mar. 23, 2006

(54) Compositions and methods comprising proteinase activated receptor antagonists

(76) Inventors: Gregory E. Agoston, Rockville, MD (US); Todd A. Hembrough, Damascus, MD (US); Theresa M. LaVallee, Rockville, MD (US); Jamshed H. Shah, Brookeville, MD (US); Lita Suwandi, Alexandria, VA (US); Anthony M. Treston, Rockville, MD (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/208,460

(22) Filed: Aug. 19, 2005

Related U.S. Application Data
(60) Provisional application No. 60/603,307, filed on Aug. 20, 2004.
(60) Provisional application No. 60/644,710, filed on Jan. 18, 2005.

(51) Int. Cl. C07D 261/18; C07D 235/30; C07D 403/02

(52) U.S. Cl. 544/162; 568/840; 544/387; 544/360; 544/370; 548/307.4; 548/248; 548/371.7

(57) ABSTRACT

Compositions and methods comprising proteinase activated receptor antagonists are provided. More particularly, the present invention relates to the use of proteins, peptides and molecules that bind to proteinase activated receptor 2, and inhibit the processes associated with the activation of that receptor. More specifically, the present invention provides novel compositions and methods for the treatment of disorders and diseases such as those associated with abnormal cellular proliferation, angiogenesis, inflammation and cancer.

### CROSS-REFERENCE TO RELATED APPLICATIONS

This application claims the benefit of U.S. Provisional Application No. 60/603,307, filed Aug. 20, 2004, and U.S. Provisional Application No. 60/644,710, filed Jan. 18, 2005, both of which are incorporated herein by reference in their entirety.

### FIELD OF THE INVENTION

The present invention relates to compositions and methods comprising proteinase activated receptor antagonists. More particularly, the present invention relates to the use of proteins, peptides and non-peptide molecules that bind to proteinase activated receptors, and inhibit the processes associated with the activation of that receptor. More specifically, the present invention provides novel compositions and methods for the treatment of disorders and diseases such as those associated with abnormal cellular proliferation, angiogenesis, inflammation and cancer.

### BACKGROUND OF THE INVENTION

Cellular proliferation is a normal ongoing process in all living organisms and is one that involves numerous factors and signals that are delicately balanced to maintain regular cellular cycles. The general process of cell division is one that consists of two sequential processes: nuclear division (mitosis), and cytoplasmic division (cytokinesis). Because organisms are continually growing and replacing cells, cellular proliferation is a central process that is vital to the normal functioning of almost all biological processes. Whether or not mammalian cells will grow and divide is determined by a variety of feedback control mechanisms, which include the availability of space in which a cell can grow, and the secretion of specific stimulatory and inhibitory factors in the immediate environment.

When normal cellular proliferation is disturbed or somehow disrupted, the results can affect an array of biological functions. Disruption of proliferation could be due to a myriad of factors such as the absence or overabundance of various signaling chemicals or presence of altered environments. Some disorders characterized by abnormal cellular proliferation include cancer, abnormal development of embryos, improper formation of the corpus luteum, difficulty in wound healing as well as malfunctioning of inflammatory and immune responses.

Cancer is characterized by abnormal cellular proliferation. Cancer cells exhibit a number of properties that make them dangerous to the host, often including an ability to invade other tissues and to induce capillary ingrowth, which assures that the proliferating cancer cells have an adequate supply of blood. One of the defining features of cancer cells is that they respond abnormally to control mechanisms that regulate the division of normal cells and continue to divide in a relatively uncontrolled fashion until they kill the host.

Angiogenesis and angiogenesis related diseases are closely affected by cellular proliferation. As used herein, the term “angiogenesis” means the generation of new blood vessels into a tissue or organ. Under normal physiological conditions, humans or animals undergo angiogenesis only in very specific restricted situations. For example, angiogenesis is normally observed in wound healing, fetal and embryonic development and formation of the corpus luteum, endometrium and placenta. The term “endothelium” is defined herein as a thin layer of flat cells that lines serous cavities, lymph vessels, and blood vessels. These cells are defined herein as “endothelial cells”. The term “endothelial inhibiting activity” means the capability of a molecule to inhibit angiogenesis in general. The inhibition of endothelial cell proliferation also results in an inhibition of angiogenesis.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Persistent, unregulated angiogenesis occurs in a multiplicity of disease states, tumor metastasis and abnormal growth by endothelial cells and supports the pathological damage seen in these conditions. The diverse pathological disease states in which unregulated angiogenesis is present have been grouped together and named, “angiogenic-dependent”, “angiogenic-associated”, or “angiogenic-related” diseases. These diseases are a result of abnormal or undesirable cell proliferation, particularly endothelial cell proliferation.

The hypothesis that tumor growth is angiogenesis-dependent was first proposed in 1971 by Judah Folkman (N. Engl. Jour. Med. 285:1182 1186, 1971). In its simplest terms the hypothesis proposes that once tumor “take” has occurred, every increase in tumor cell population must be preceded by an increase in new capillaries converging on the tumor. Tumor “take” is currently understood to indicate a prevascular phase of tumor growth in which a population of tumor cells occupying a few cubic millimeters volume and not exceeding a few million cells, survives on existing host microvessels. Expansion of tumor volume beyond this phase requires the induction of new capillary blood vessels. For example, pulmonary micrometastases in the early prevascular phase would be undetectable except by high power microscopy on histological sections. Further indirect evidence supporting the concept that tumor growth is angiogenesis dependent is found in U.S. Pat. Nos. 5,639,725, 5,629,327, 5,792,845, 5,733,876, and 5,854,205, all of which are incorporated herein by reference.

Thus, it is clear that cellular proliferation, particularly endothelial cell proliferation, and most particularly angiogenesis, plays a major role in the metastasis of a cancer. If this abnormal or undesirable proliferation activity could be repressed, inhibited, or eliminated, then the tumor, although present, would not grow. In the disease state, prevention of abnormal or undesirable cellular proliferation and angiogenesis could avert the damage caused by the invasion of the new microvascular system. Therapies directed at control of the cellular proliferative processes could lead to the abrogation or mitigation of these diseases.

Recently, studies have been conducted that correlate abnormal proteinase activated receptor activity with certain disorders and diseases. Of particular interest is proteinase activated receptor-2 which has been discovered to be associated with disorders such as inflammation, angiogenesis, and sepsis. Although several attempts have been made, no effective antagonists of proteinase activated receptor-2 have been identified.

What is needed are compositions and methods that can inhibit abnormal or undesirable cellular function, especially functions that are associated with undesirable cellular proliferation, angiogenesis, inflammation and cancer. The compositions should comprise proteins, peptides or non-peptide molecules that overcome the activity of endogenous proteinase activated receptor ligands and prevent the activation of proteinase activated receptors thereby inhibiting the development of abnormal physiological states associated with inappropriate proteinase activated receptor activation. Finally, the compositions and methods for inhibiting proteinase activated receptor activation should preferably be non-toxic and produce few side effects.

### SUMMARY OF THE INVENTION

Compositions and methods are provided that are effective in inhibiting abnormal or undesirable cell function, particularly cellular activity and proliferation related to angiogenesis, neovascularization, inflammation, tumor growth, sepsis, neurogenic and inflammatory pain, asthma and post operative ileus. The compositions comprise a naturally occurring or synthetic protein, peptide, protein fragment or non-peptide molecule containing or mimicking the action of all or an active portion of a ligand that binds proteinase activated receptors, optionally combined with a pharmaceutically acceptable carrier.

Representative ligands or antagonists useful for the present invention comprise proteins, peptides and molecules that bind proteinase activated receptors, such as, but not limited to, proteinase activated receptor 1 (PAR-1), proteinase activated receptor 2 (PAR-2), proteinase activated receptor 3 (PAR-3), or proteinase activated receptor 4 (PAR-4). Preferred ligand compositions of the present invention, include, but are not limited to, peptides comprising LIGK (ENMD-1005) (SEQ ID NO:1), LIGKV (ENMD-1007) (SEQ ID NO:2), KGIL (SEQ ID NO:3), KGI (SEQ ID NO:4), AGI (SEQ ID NO:5), IGA (SEQ ID NO:6), KGA (SEQ ID NO:7), KGA (SEQ ID NO:8), KAI (SEQ ID NO:9), IAK (SEQ ID NO:10), RGI (SEQ ID NO:11), IGR (ENMD-1023) (SEQ ID NO:12), Dab-GI (Dab=diamino butanoic acid) (SEQ ID NO:13), Dap-GI (Dap=diamino proprionic acid) (SEQ ID NO:14), IG-Dab (ENMD-1024) (SEQ ID NO:15), IG-Dap (ENMD-1025) (SEQ ID NO:16), LIG-Dab (ENMD-1026) (SEQ ID NO:17), Dab-GIL (SEQ ID NO:18), LIG-Dap (ENMD-1027) (SEQ ID NO:19), Dap-GIL (SEQ ID NO:20), LIG-Orn (Orn=ornithine, ENMD-1028) (SEQ ID NO:21), Orn-GIL (SEQ ID NO:22), Orn-GI (SEQ ID NO:23), IG-Orn (ENMD-1029) (SEQ ID NO:24), LIG (4-amino-phenylalanine) (ENMD-1030) (SEQ ID NO:25), LIG (2-amino-glycine) (ENMD-1031) (SEQ ID NO:26), dL-dI-dG-dK (d=D-amino acids) (ENMD-1032) (SEQ ID NO:27), dI-dG-dK (ENMD-1383) (SEQ ID NO:28), LIG-dK (ENMD-1384) (SEQ ID NO:29), IG-dK (ENMD-1087) (SEQ ID NO:30), IGK-amide (ENMD-1021) (SEQ ID NO:31), LIGR (ENMD-1022) (SEQ ID NO:32), LIGD (ENMD-1045) (SEQ ID NO:33), LIGE (ENMD-1046) (SEQ ID NO:34), LIGN (ENMD-1047) (SEQ ID NO:35), LIGQ (ENMD-1048) (SEQ ID NO:36), LIGS (ENMD-1049) (SEQ ID NO:37), LIGT (ENMD-1050) (SEQ ID NO:38), LIGY (ENMD-1051) (SEQ ID NO:39), LIPK (ENMD-1052) (SEQ ID NO:40), LPGK (ENMD-1053) (SEQ ID NO:41), LIGH (ENMD-1054) (SEQ ID NO:42), L-Statine-K (ENMD-1056) (SEQ ID NO:43), L-Statine-GK (ENMD-1057) (SEQ ID NO:44), L-Nipecotic acid-K (ENMD-1058) (SEQ ID NO:45), L-Nipecotic acid-GK (ENMD-1059) (SEQ ID NO:46), L-Hydroxypiperidine-K (ENMD-1060) (SEQ ID NO:47), L-Hydroxypiperidine-GK (ENMD-1061) (SEQ ID NO:48), L-Imidazolidine-K (ENMD-1062) (SEQ ID NO:49), L-Imidazolidine-GK (ENMD-1063) (SEQ ID NO:50), and LIGM (ENMD-1064) (SEQ ID NO:51), and various molecules and described below. Also contemplated within the scope of this invention are ligands and antagonists that comprise functional and structural derivatives and equivalents of the above-listed molecules.

Preferably, the protein, peptide, protein fragment or molecule of the present invention contains or mimics the action of all or an active portion of the above identified ligands and antagonists. The term “active portion”, as used herein, means a portion of a protein, peptide or molecule that inhibits proteinase activated receptor activation. Also included in the present invention are homologs, peptides, protein fragments, or combinations thereof of the above-identified ligands and antagonists, that inhibit proteinase activated receptor activity.

It is believed that by inhibiting proteinase activated receptor activity, the methods and compositions described herein are useful for inhibiting diseases and disorders associated with abnormal proteinase activated receptor activity. The methods provided herein for treating diseases and processes mediated by proteinase activated receptors, such as inflammation and cancer, involve administering to a human or animal the composition described herein in a dosage sufficient to inhibit proteinase activated receptor activity, particularly PAR-2 activity. The methods are especially useful for treating or repressing the growth of tumors, particularly by inhibiting angiogenesis and for reducing inflammation and inflammatory responses.

Accordingly, it is an object of the present invention to provide methods and compositions for treating diseases and processes that are mediated by abnormal or undesirable proteinase activated receptor activity.

Another object of the present invention is to provide methods and compositions for inhibiting abnormal or undesirable cell function, cellular activity and proliferation particularly related to angiogenesis, neovascularization, inflammation, conditions related to inflammation, tumor growth, tumor metastasis, sepsis, neurogenic and inflammatory pain, asthma and post operative ileus.

It is another object of the present invention to provide methods and compositions for treating or repressing the growth of a cancer or a tumor metastasis.

It is yet another object of the present invention to provide methods and compositions for therapy of cancer that has minimal side effects.

It is another object of the present invention to provide methods and compositions for treating diseases and processes that are mediated by angiogenesis.

It is another object of the present invention to provide methods and compositions for treating or repressing inflammation, inflammatory responses and inflammatory diseases.

It is yet another object of the present invention to provide methods and compositions for therapy of inflammation that has minimal side effects.

It is another object of the present invention to provide methods and compositions for treating diseases and processes that are mediated by inflammation or inflammatory responses, including, but not limited to, acute inflammation, chronic inflammation, rheumatoid arthritis, dermatitis, inflammatory bowel disease, inflammatory bowel syndrome, asthma, sepsis, neurogenic pain, and dermatitis.

Yet another object of the present invention is to provide methods and compositions comprising the use of proteins, peptides, molecules, active fragments and homologs thereof that inhibit proteinase activated receptor activity.

Another object of the present invention is to provide methods and compositions for treating diseases and processes that are mediated by angiogenesis by administrating antiangiogenic compounds comprising ligands that bind proteinase activated receptor activity.

It is a further object of the present invention to provide methods and compositions for treating diseases and processes that are mediated by abnormal proteinase activated receptor activity.

It is another object of the present invention to provide methods and compositions for diagnosing diseases and disorders by measuring abnormal proteinase activated receptor activity.

It is still another object of the present invention to provide compositions comprising ligands that bind proteinase activated receptors wherein the compositions further comprise pharmaceutically acceptable carriers.

Yet another object of the present invention is to provide methods and compositions comprising ligands that bind proteinase activated receptors wherein the compositions further comprise pharmaceutically acceptable carriers that may be administered intranasal, intramuscularly, intravenously, transdermally, orally, topically, vaginally, rectally, or subcutaneously.

It is yet another object of the present invention to provide compositions and methods for treating diseases and processes that are mediated by angiogenesis including, but not limited to, hemangioma, solid tumors, blood borne tumors, leukemia, tumor metastasis, telangiectasia, psoriasis, scleroderma, pyogenic granuloma, myocardial angiogenesis, atherosclerosis, Crohn's disease, plaque neovascularization, arteriovenous malformations, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, endometriosis, ovulation, menstruation, placentation, and cat scratch fever.

These and other objects, features and advantages of the present invention will become apparent after a review of the following detailed description of the disclosed embodiment and the appended claims.

### BRIEF DESCRIPTION OF THE FIGURES

FIG. 1 provides a schematic showing a proposed interaction of an antagonist with activated PAR receptor.

FIG. 2A shows calcium mobilization curves of the PAR-2 agonist peptide (also referred to as AP2, or P2AP, or P2P) SLIGKV (ENMD-1003) (SEQ ID NO:52) compared with two truncated molecules LIGK (ENMD-1005) (SEQ ID NO:1) and LIGKV (ENMD-1007) (SEQ ID NO:2). FIG. 2B shows the results of an in vitro assay demonstrating PAR-2 Ca²⁺ signaling in response to PAR-2 activating peptide and several alanine-substituted analogs. FIG. 2C shows the results of an in vitro Ca²⁺ assay demonstrating PAR-2 signaling in response to AP2 and its truncated forms and alanine substituted analogs.

FIG. 3 shows a representative dosing study where increasing concentrations of LIGK (ENMD-1005) (SEQ ID NO:1) were used to block PAR-2 agonist peptide (AP2) signaling.

FIG. 4 provides a graph showing the results of an in vitro Ca²⁺ signaling inhibition study of AP2 stimulated HT29 cells in the presence of LIGK (ENMD-1005) (SEQ ID NO:1) or LIGKV (ENMD-1007) (SEQ ID NO:2).

FIG. 5 provides a graph showing the effect of LIGK (ENMD-1005) (SEQ ID NO:1) on PAR-2 (AP2), PAR-1 (AP1) and ATP signaling.

FIG. 6 provides the effect of LIGK (ENMD-1005) (SEQ ID NO:1) on a PAR-2 murine footpad edema model.

FIG. 7 shows the results of an in vivo Matrigel angiogenesis assay demonstrating the inhibitory effect of LIGK (ENMD-1005) (SEQ ID NO:1).

FIG. 8 provides a graph showing a decrease in AP2 stimulated Ca²⁺ signaling in the presence of ENMD-1068 in vitro.

FIG. 9 shows the effect of ENMD-1068 on ATP and AP2 Ca²⁺ signaling in vitro.

FIG. 10 provides a flow chart showing an example of a peptidomimetic design approach.

FIG. 11 shows attenuation of arthritis in the presence of LIGK (ENMD-1005) (SEQ ID NO:1), and ENMD-1068 in a mouse model.

FIG. 12 shows prevention of weight loss in mice in the presence of LIGK (ENMD-1005) (SEQ ID NO:1) in this same arthritis model.

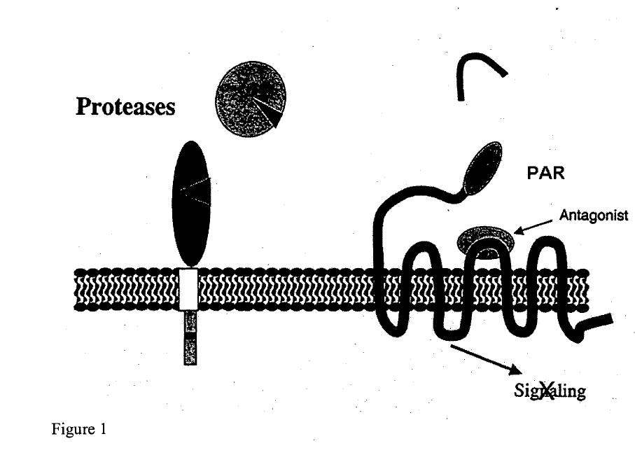

### DETAILED DESCRIPTION

The following description includes the best presently contemplated mode of carrying out the invention. This description is made for the purpose of illustrating the general principles of the inventions and should not be taken in a limiting sense. The entire text of the references mentioned herein are hereby incorporated in their entireties by reference, including U.S. Provisional Application Ser. No. 60/391,655 filed Jun. 26, 2002, U.S. Provisional Application Ser. No. 60/398,662 filed Jul. 26, 2002, U.S. Provisional Application Ser. No. 60/458,095 filed Mar. 27, 2003 and U.S. Provisional Application Ser. No. 60/466,296 filed Apr. 29, 2003, as well as U.S. application Ser. No. 10/608,886 filed Jun. 26, 2003 and Ser. No. 10/833,252 filed Apr. 27, 2004.

Proteinase activated receptor-2 (PAR-2) is a seven transmembrane G-protein coupled receptor (GPCR) which signals in response to the proteolytic activity of trypsin, tryptase, matriptase, the tissue factor (TF)/factor VIIa (fVIIa) complex and other proteases, including, but not limited to, neutrophil protease-3. Proteolytic cleavage of the amino terminus results in the unveiling of a new amino terminus that activates the receptor through a tethered peptide ligand mechanism; essentially the new terminus becomes the ligand which inserts into the ligand binding pocket of the receptor. The short synthetic activating peptide (known variously as AP2 or P2AP or P2P), SLIGKV (ENMD-1003) (SEQ ID NO:52) (human), SLIGRL-NH₂ (mouse) (SEQ ID NO:53)) activates the receptor. Upon binding of the ligand, there is an increase in intracellular calcium concentration indicating activation of the receptor.

Several studies have demonstrated that PAR-2 is involved in angiogenesis, neovascularization and inflammation. PAR-2 has also been associated with pain transmission, tissue injury and regulation of cardiovascular function. For example, Milia et al. discuss the wide expression of PAR-2 in the cardiovascular system, mediation of endothelial cell mitogenesis in vitro by PAR-2, and promotion of vasodilation and microvascular permeability in vivo by PAR-2: all of these steps are regarded as essential steps in angiogenesis. (Milia et al. Circulation Research Vol. 91 (4) 2002 pp. 346-352, which is incorporated herein by reference in its entirety). Milia et al. further discuss upregulation of PAR-2 expression by cytokines, including tumor necrosis factor-a, interleukin-b, and lipopolysaccharide, all thought to be involved in inflammation (ibid).

In addition, recent studies have shown that PAR-2 activation mediates neurogenic inflammation and nociception, illustrating that in some cases, activation of PAR-2 on neurons leads to the generation of proinflammatory cytokines, and a panoply of inflammatory signals. PAR-2 has also been shown to play an essential role in the onset of chronic inflammatory diseases such as rheumatoid arthritis.

Based on the current knowledge of PAR activity in abnormal physiological states, it is believed that PAR activity and in particular PAR-2 activity is associated with numerous disorders and diseases including, but not limited to, angiogenesis, neovascularization, inflammation, tumor growth, sepsis, neurogenic and inflammatory pain, asthma and post operative ileus.

We have previously shown that the proteolytic activity of the PAR-2 agonist TF/fVIIa promotes tumor growth and angiogenesis independently of its role in coagulation (Hembrough et al, Blood 103:3374-3380). Further characterization and analysis of the role of PAR-2 and its involvement in disease has been difficult, because until now, no specific antagonists of PAR-2 had been identified. Here we describe specific antagonists of PAR-2 signaling. In vivo, these PAR-2 antagonists are inhibitors of angiogenesis, tumor growth and inflammatory diseases. Since previous studies by the inventors suggested a possible role for PAR-2 in tumor growth and angiogenesis, these inhibitors were further assessed to determine if they could inhibit tumor growth or angiogenesis. In vivo treatment with these PAR-2 inhibitors results in inhibition of both angiogenesis and tumor growth. Thus, these inhibitor studies demonstrate that PAR-2 activity plays a role in regulating angiogenesis and tumor growth. These data describing potent and specific antagonists of PAR-2 signaling promise to be powerful tools for the study of PAR-2 physiology in normal and pathological processes and for amelioration of disease processes mediated by PAR-2.

The studies described herein provide the first identification of PAR-2 antagonists. Numerous reports have been published demonstrating important physiological functions of PAR-2. These activities include nociception, acute and chronic inflammation, dermatitis, rheumatoid arthritis, asthma, and neurogenic pain. In each of these studies mention is made of the need for specific PAR-2 antagonists and their great value in the future characterization of this receptor.

Although other studies claim to describe methods that involve inhibiting PAR-2 activity, none of them actually identify specific antagonists. For example, one such study focuses instead on blocking proteolytic cleavage of the PAR-2 amino terminal by trypsin, tryptase, matriptase or the tissue factor (TF)/factor VIIa (fVIIa) complex (see for example WO 01/52883 A1). Such studies acknowledge the need for PAR-2 antagonists, but fail to define any specific inhibitors or provide any guidance with regard to potential structures for such peptides, proteins or molecules. The present inventors have successfully identified specific inhibitors of PAR-2, as well as certain protein/peptide structures that enable the design and elucidation of PAR-2 antagonists.

As discussed above, PARs are a family of GPCRs that function as sensors of thrombotic or inflammatory proteinase activity. Knockout mice lacking the PAR-2 receptor demonstrated little joint swelling or tissue damage in an adjuvant monoarthritis model of chronic inflammation, thereby confirming the role of PAR-2 in inflammation. In another experiment, the inventors showed that the tissue factor coagulation pathway was required for the growth of both primary and metastatic tumors. This required the activity of TF/fVIIa complex, but not fXa, which is the normal, physiological target of TF/fVIIa activity. Accordingly, though not wishing to be bound by the following theory, it is believed that in abnormal physiological states, the TF/fVIIa complex is targeting something other than fXa, and based on the studies herein, the inventors believe that the target is PAR-2.

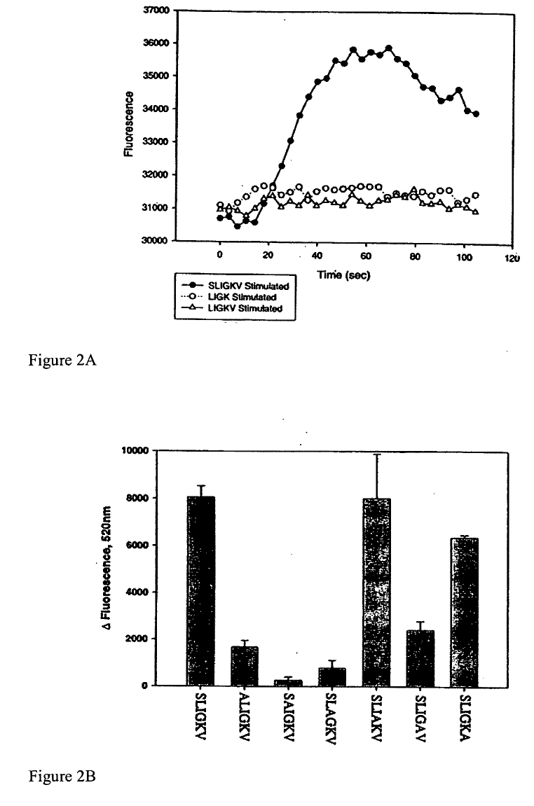

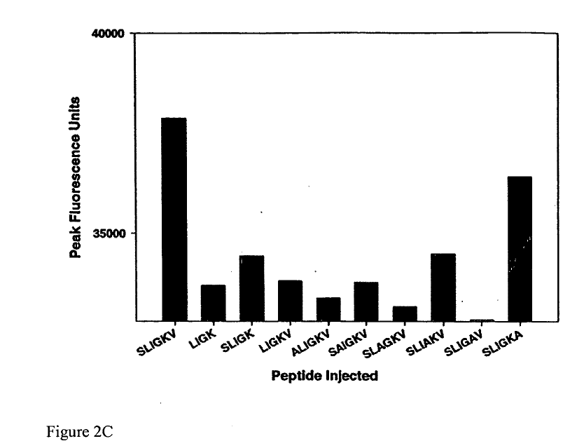

In order to design a peptide antagonist for PAR-2, the inventors first mapped the signaling activity of the agonist peptide, SLIGKV (ENMD-1003) (SEQ ID NO:52) (this signaling peptide is also variously known as P2AP or 2AP or AP2 in the scientific literature) which was either truncated or monosubstituted with alanine. This was done in order to exclude those peptides that retained signaling activity, and would desensitize cells in inhibition studies. FIG. 2A shows calcium mobilization curves of the PAR-2 agonist SLIGKV (ENMD-1003) (SEQ ID NO:52) compared with two truncated molecules LIGK (ENMD-1005) (SEQ ID NO:1) and LIGKV (ENMD-1007) (SEQ ID NO:2). Neither truncated molecule was able to induce calcium mobilization, in contrast with SLIGKV (ENMD-1003) (SEQ ID NO:52), which demonstrates the typical spike of calcium release followed by degradation of signal. Similar studies were performed on alanine substituted SLIGKV (ENMD-1003) (SEQ ID NO:52) peptides (FIGS. 2B and 2C). It was found that substitution of SLIGKV (SEQ ID NO:52) at S, L, I, or K abrogated or significantly diminished signaling activity, while two substituted peptides, SLIAKV (ENMD-1011) (SEQ ID NO:54) and SLIGKA (ENMD-1013) (SEQ ID NO:55) demonstrated robust signaling activity.

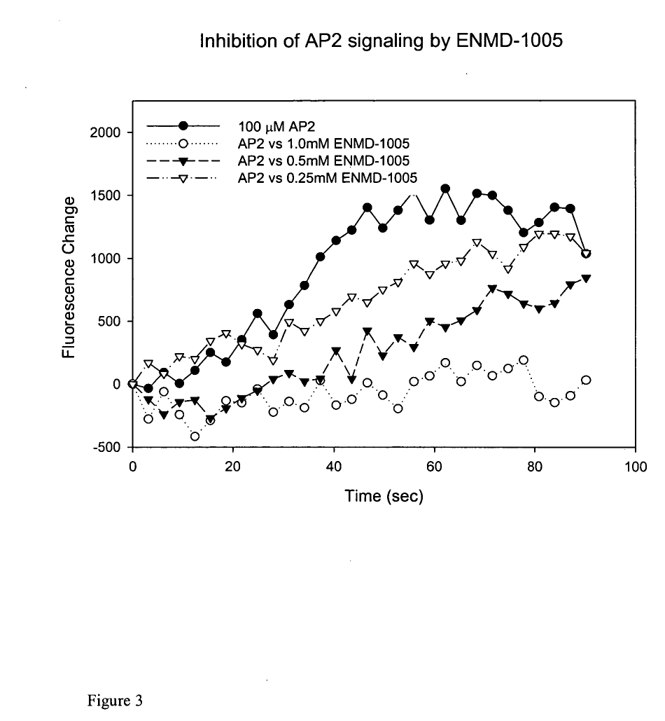

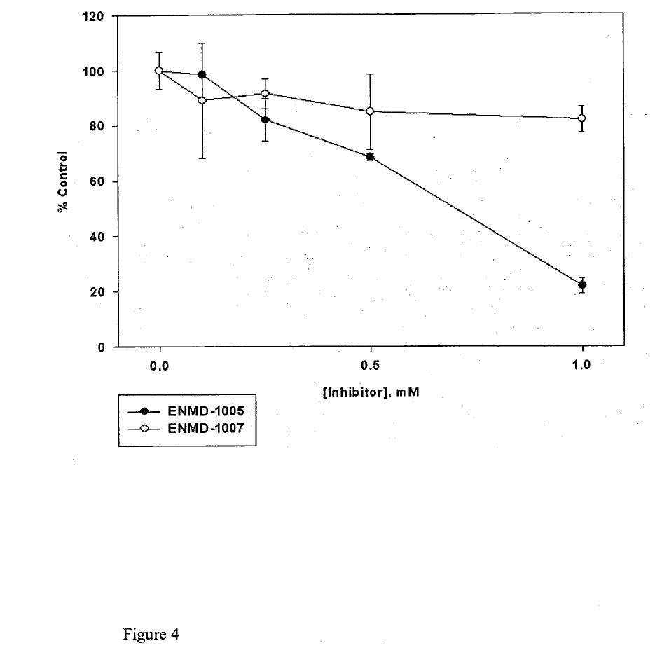

The inventors hypothesized that one of these peptides which lack PAR-2 signaling activity might function instead as a PAR-2 antagonist, since it could retain the ability to bind to the PAR-2 receptor while lacking the ability to signal. In this way, such a peptide might function as a competitive inhibitor, since it could block or displace the endogenous agonist peptide from binding and signaling. In order to assess the potential of these peptides to block PAR-2 signaling, cells were pretreated with potential antagonist peptides for a predetermined amount of time and were subsequently treated with P2AP. Two of the SLIGKV (ENMD-1003) (SEQ ID NO:52) derived peptides demonstrated antagonist activity, LIGK (ENMD-1005) (SEQ ID NO:1) and LIGKV (ENMD-1007) (SEQ ID NO:2). FIG. 3 shows a representative antagonist study where LIGK (ENMD-1005) (SEQ ID NO:1) was used to block AP2 signaling. In this study, a concentration of 1 mM LIGK (ENMD-1005) (SEQ ID NO:1) completely blocked the signaling of 100 uM SLIGKV (ENMD-1003) (SEQ ID NO:52). In similar studies comparing the activity of LIGK (ENMD-1005) (SEQ ID NO:1) with LIGKV (ENMD-1007) (SEQ ID NO:2) it was found that the LIGK (ENMD-1005) (SEQ ID NO:1) peptide is a more potent inhibitor of PAR-2 signaling (IC50<0.5 mM), compared to LIGKV (ENMD-1007) (SEQ ID NO:2) (FIG. 4).

Additional peptides having PAR antagonist activity include, but are not limited to, KGIL (SEQ ID NO:3), KGI (SEQ ID NO:4), AGI (SEQ ID NO:5), IGA (SEQ ID NO:6), KGA (SEQ ID NO:7), KGA (SEQ ID NO:8), KAI (SEQ ID NO:9), IAK (SEQ ID NO:10), RGI (SEQ ID NO:11), IGR (ENMD-1023) (SEQ ID NO:12), Dab-GI (Dab=diamino butanoic acid) (SEQ ID NO:13), Dap-GI (Dap=diamino proprionic acid) (SEQ ID NO:14), IG-Dab (SEQ ID NO:15), IG-Dap (ENMD-1025) (SEQ ID NO:16), LIG-Dab (ENMD-1026) (SEQ ID NO:17), Dab-GIL (SEQ ID NO:18), LIG-Dap (ENMD-1027) (SEQ ID NO:19), Dap-GIL (SEQ ID NO:20), LIG-Orn (Orn=ornithine, ENMD-1028) (SEQ ID NO:21), Orn-GIL (SEQ ID: 22), Orn-GI (SEQ ID NO:23); IG-Orn (ENMD-1029) (SEQ ID NO:24), LIG (4-amino-phenylalanine) (ENMD-1030) (SEQ ID NO:25), LIG (2-amino-glycine) (ENMD-1031) (SEQ ID NO:26), dL-dI-G-dK (d=D-amino acids) (ENMD-1032) (SEQ ID NO:27), dI-G-dK (ENMD-1383) (SEQ ID NO:28), LIG-dK (ENMD-1384) (SEQ ID NO:29), IG-dK (ENMD-1087) (SEQ ID NO:30), IGK-amide (ENMD-1021) (SEQ ID NO:31), LIGR (ENMD-1022) (SEQ ID NO:32), LIGD (ENMD-1045) (SEQ ID NO:33), LIGE (ENMD-1046) (SEQ ID NO:34), LIGN (ENMD-1047) (SEQ ID NO:35), LIGQ (ENMD-1048) (SEQ ID NO:36), LIGS (ENMD-1049) (SEQ ID NO:37), LIGT (ENMD-1050) (SEQ ID NO:38), LIGY (ENMD-1051) (SEQ ID NO:39), LIPK (ENMD-1052) (SEQ ID NO:40), LPGK (ENMD-1053) (SEQ ID NO:41), LIGH (ENMD-1054) (SEQ ID NO:42), L-Statine-K (ENMD-1056) (SEQ ID NO:43), L-Statine-GK (ENMD-1057) (SEQ ID NO:44), L-Nipecotic Acid-K (ENMD-1058) (SEQ ID NO:45), L-Nipecotic acid-GK (ENMD-1059) (SEQ ID NO:46), L-Hydroxypiperidine-K (ENMD-1060) (SEQ ID NO:47), L-Hydroxypiperidine-GK (ENMD-1061) (SEQ ID NO:48), L-Imidazolidine-K (ENMD-1062) (SEQ ID NO:49), L-Imidazolidine-GK (ENMD-1063) (SEQ ID NO:50), and LIGM (ENMD-1064) (SEQ ID NO:51).

Additional molecules which show PAR antagonist activity include, but are not limited to: ENMD-1033, ENMD-1034, ENMD-1035, ENMD-1036, ENMD-1037, ENMD-1038, ENMD-1039, ENMD-1040, ENMD-1041, ENMD-1065, ENMD-1070, ENMD-1075, ENMD-1066, ENMD-1071, ENMD-1076, ENMD-1067, ENMD-1072, ENMD-1077, ENMD-1068, ENMD-1073, ENMD-1078, ENMD-1069, ENMD-1074 and ENMD-1079.

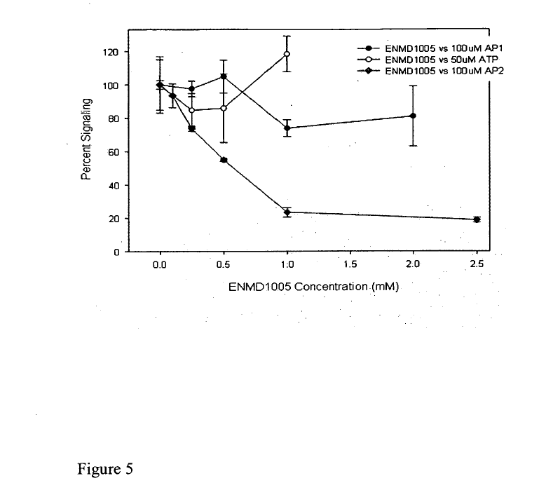

In order to demonstrate that LIGK (ENMD-1005) (SEQ ID NO:1) is a specific inhibitor of PAR-2 signaling, activation studies were performed with ATP and the PAR-1 activation peptide, SFLLRN (ENMD-1014) (SEQ ID NO:56), on cells that were pretreated with LIGK (ENMD-1005) (SEQ ID NO:1). Both of these molecules signal through GPCRs, and PAR-1 is highly homologous to PAR-2, to the degree that the PAR-1 agonist peptide can signal through PAR-2 at high concentrations. In both cases, the PAR-2 antagonist LIGK (ENMD-1005) (SEQ ID NO:1) had no inhibitory effect on either PAR-1 or ATP signaling (FIG. 5).

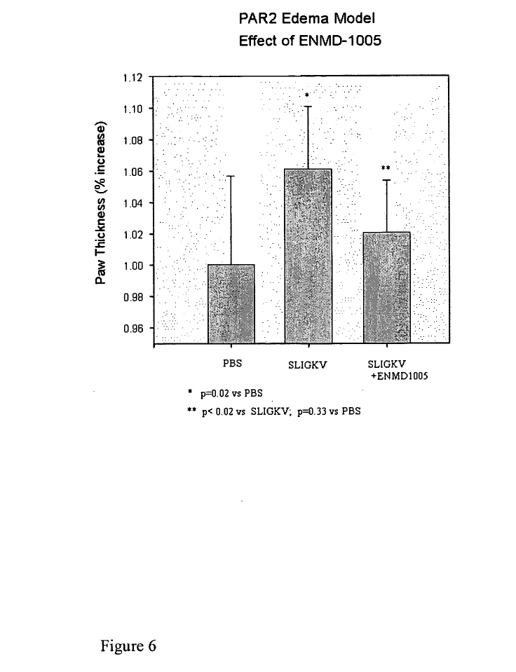

The inventors next assessed whether the LIGK (ENMD-1005) (SEQ ID NO:1) peptide had in vivo PAR-2 antagonistic activity. This was studied using a mouse edema model where vascular permeability was induced by the PAR-2 agonist peptide. In this model, the PAR-2 activating peptide induces severe edema as previously reported (FIG. 6). This vascular response was blocked by co-treatment with the PAR-2 antagonist LIGK (ENMD-1005) (SEQ ID NO:1) (FIG. 6). Thus, LIGK (ENMD-1005) (SEQ ID NO:1) functions in vivo to block PAR-2 signaling.

In order to confirm the role of PAR-2 in tumor angiogenesis and inflammation physiology and to develop new agents for inhibition of PAR-2 and other PARs, the present inventors designed and synthesized novel antagonists based on the structure of the LIGK antagonist peptide, generally comprising structures that have a basic or other polar or hydrogen-bonding portion in one region of the molecule (for example a chemical moiety mimicking lysine) and a linker attaching that moiety to a hydrophobic moiety on another portion of the molecule (for example a chemical moiety mimicking leucine). The general criteria for each component is as follows. The hydrophobic moiety can be either substituted or unsubstituted, straight or branched, aliphatic and may contain carbocyclic or heteroatom-containing rings such as listed below and may be saturated or unsaturated. The polar or hydrophilic moiety would preferably have as a hydrophilic or polar residue a moiety including, but not limited to, alcohol, amine, acid, guanine, ester or amide functional groups, and can include linear or branched, saturated or unsaturated, carbocyclic or heterocyclic rings.

The linker can comprise any chemical moiety which structurally, spatially, chemically and/or electronically generally mimics the spacing provided by the Ile and Gly residues in LIGK (ENMD-1005) (SEQ ID NO:1). Examples of possible linkers include, but are not limited to, saturated, unsaturated or aromatic ring systems, linear or branched unsaturated or saturated hydrocarbon chains, sugars, nucleotides or nucleosides, single or multiple ring unsaturated or saturated carbocycles or heterocycles. Linkers could include one or more heteroatoms (including, but not limited to, halides, nitrogen, oxygen, sulfur, silicon, selenium, or phosphorous), linkers could be non-cyclic, the terminal R groups could be bound to any position on the linker, linkers could have heteroatom-containing substituent groups (including, but not limited to, imidazoles, aminos, arginyls, aminophenyls, pyridyls, thiols, alcohols, acids, esters, halides or amides), and linkers can have aliphatic groups other than simple linear or branched hydrocarbon chains. A partial list of possible linkers includes, but is not limited to, substituted or unsubstituted phenyls, bi-aryls (such as bi-phenyls), azetidines, benzyls, saturated or unsaturated, branched or linear, hydrocarbons (including alkanes, alkenes, or alkynes), sugars (including glucuronic acids, glucosamines, and glucoses), polyols polyamines, phosphates, sulfates, sulfonates, phosphoramides, cyclopropanes, cyclobutanes, cyclopentanes, cyclohexanes, cycloheptanes, furans, thiophenes, 2H-pyrroles, pyrroles, 2-pyrrolines, 3-pyrrolines, pyrrolidines, 1,3-dioxanes, oxazoles, oxazolines, imidazoles, 1-imidazolines, imidazolidines, pyrazoles, 2-pyrazolines, 3-pyrazolines, pyrazolidines, isoxazoles, isothiazoles, 1,2,3-oxadiazoles, 1,2,3-triazoles, 1,3,4-thiadiazoles, 2H-pyrans, thiazolidines, 4H-pyrans, pyridines, piperidines, 1,2-dioxanes, 1,4-dioxanes, 1,2-morpholines, 1,3-morpholines, 1,4-morpholines, 1,2-dithianes, 1,3-dithianes, 1,4-dithianes, 1,2-thiomorpholines, 1,3-thiomorpholines, 1,4-thiomorpholines, pyridazine, pyrimidines, pyrazines, 1,2-piperazines, 1,3-piperazines, 1,4-piperazines, sultams, thiazoles, 1,3,5-triazines, triazoles, tetrazoles, 1,3,5-trithianes, 1,2,3,4-tetrahydro-1,3-diazines, indolizines, indoles, isoindoles, 3H-indoles, indolines, benzo[b]furans, benzo[b]thiophenes, 1H-indazoles, benzimidazoles, benzimidazolones, benzthiazoles, benzthioxoles, purines, 4H-quinolizines, quinolines, isoquinolines, cinnolines, phthalazines, quinazolines, quinoxalines, 1,8-naphthyridines, pteridines, quinuclidines, carbazoles, acridines, phenazines, phenothiazines, phenoxazines, indenes, naphthalenes, azulenes, fluorenes, anthracenes, norboranes, adamantanes, b-carbolines, perimidines, furazans, phenanthridines, phenanthrolines, phenarsazines, chromans, and isochromans. The hydrophobic and polar moieties can be attached to the linker either through heteroatoms or any of the carbon atoms on the linker where chemically possible.

The hydrophobic and the polar moieties and the linker moieties can be further substituted. Such substitutions can be made for reasons including to enhance binding to or affinity for the PAR agonist or antagonist binding region, to enhance or modify specificity for an individual PAR compared to other receptors or other binding proteins, to modify metabolic characteristics, to modify pharmacological properties, to modify physicochemical properties (including, but not limited to, water solubility, partition coefficients, membrane permeability, polar surface area, and regional polarity or electronic or hydrophobic or surface area parameters), to modify metabolism (for example substitution of metabolically labile protons by halogen atoms), to modify absorption characteristics for the chosen route of administration (including, but not limited to, oral, systemic, nasal, inhalation, buccal, rectal, vaginal, topical, and transdermal), to improve chemical and biological stability, to improve the ability of the molecule to be formulated for the desired route of administration, to modify the ability of the molecule to act as a substrate for enzymes involved with drug metabolism and excretion (including, but not limited to, cytochromes including CYPs, transferases including UDPGTs and GSTs, and transporters including MDR), to modify the biodistribution, clearance or half-life of the molecule, to modify toxicities, and to modify the targeting of the molecule to desired sites of action.

The processes by which these changes are made to molecules of interest are well known to those skilled in the fields of medicinal chemistry, drug discovery, and drug development, and include, but are not limited to, combinatorial and parallel chemistry, medicinal chemistry, in silico modeling, computer-aided drug design, in silico modeling of absorption, distribution, metabolism, elimination or toxicology, and modeling using predictive techniques including, but not limited to, in silico pharmacophores, QSAR and CoMFA.

The methods by which improvements in or modifications to properties of molecules are measured or tested are well known to those skilled in the fields of medicinal chemistry, drug design, drug development, toxicology, physiology and pharmacology. Examples of these methods include, but are not limited to, PAMPA or CaCo2 assessment of permeability; in vivo, in vitro, and ex vivo testing of pharmacology including, but not limited to, receptor activation and/or signaling, reduction in angiogenesis, tumor growth, tumor metastasis, or inflammation; in vivo, in vitro or ex vivo testing of binding; in vivo, in vitro, and ex vivo assessment of toxicology; in vitro metabolism assays using cells, cell extracts, or isolated drug metabolism enzymes; in vivo determinations of absorption, distribution, metabolism, elimination or toxicology; cardiac toxicity testing using hERG ion channel assays; formulation studies; and pre-clinical and clinical evaluations in humans or other animal species.

The moieties or components of the PAR antagonists can be assembled using a number of synthetic approaches using appropriate protecting groups. Approaches for linking moieties or components include but are not limited to amides, amines, C—C bonds, ethers, and esters. These approaches are given as examples only, and are not limiting. These and other approaches are well known to those skilled in the art of organic chemistry, medicinal chemistry or drug design. For example, where the components are linked by an amide functionality, peptide or amide coupling reactions can be used. Such coupling reagents include, but are not limited to, 1,3-dicyclohexyl carbodiimide, 1-ethyl-3-(3-dimethylaminopropyl)-carbo-diimide, 1-hydroxy-benzotriazole and N,N-diisopropylethyl amine or carbonyl diimidizole. Attachments to carbocyclic or heterocyclic rings can be accomplished by use of enolate or Wittig type chemistry using the appropriate carbonyl precursors. Heterocycles including pyrazoles can be formed with desired substitutions in place through cyclization reactions such as described by Stauffer et al., in Bioorganic and Medicinal Chemistry, volume 9, pages 141-150 (2001) which is incorporated herein by reference in its entirety. Several of the heterocycles can be synthesized by coupling the appropriately substituted precursors to generate the heterocyclic ring (March and Smith, Advanced Organic Chemistry, Wiley Interscience, New York, N.Y., 2001; Sainsbury, Malcom, Heterocyclic Chemistry, Royal Society of Chemistry, Cambridge, UK, 2001; Davies, D, Aromatic Heterocyclic Chemistry, Oxford University Press, Oxford, UK, 2004; Jie Jack Lie, Ed., Name Reactions in Heterocyclic Chemistry, Wiley, New York, N.Y., 2004), all of which are incorporated herein by reference in their entirety. These and other texts and the chemical literature can also be used to aid in functionalizing existing carbocyclic or heterocyclic rings. Grignard or lithium reagents can be prepared to couple components together via halogen substituted moieties. Aromatic halogens can also undergo Friedel-Crafts acylations or alkylations to give coupled heterocycles. Many name reactions that can be used to couple the individual components are known to those skilled in the art and are listed in texts such as: March and Smith, Advanced Organic Chemistry, Wiley Interscience, New York, N.Y., 2001; Carey and Sundburg, Advanced Organic Chemistry, Part B. Reactions and Synthesis, Fourth Ed., Kluwer Academic/Plenum Publishing, New York, N.Y. 2001; Jie Jack Li, Name Reactions, Springer, New York, N.Y., 2002; Hassner and Stumer, Organic Synthesis Based on Name Reactions, Second Ed., Pergamon Press, New York, N.Y., 2002; and Mundy and Ellerd, Name Reactions and Reagents in Organic Synthesis, Wiley Interscience, New York, N.Y., 1988, all of which are incorporated herein by reference in their entirety. Those skilled in the art understand that various protection groups can be used to ensure the synthesis of the desired product. Protection groups commonly used include, but are not limited to, ester, amide, carbamate, benzyl, t-Boc, trityl, and Cbz groups and are described in texts including Greene and Wuts, Protective Groups in Organic Synthesis; 3^{rd} Ed. Wiley Interscience, New York, N.Y., 1999, and Kocienski, Protective Groups, 3^{rd} Ed. Verlag, NY, N.Y. 2003, all of which are incorporated herein by reference in their entirety. It is well understood by those skilled in the art that acids and bases can be prepared either as salts or in un-ionized forms (conjugate acids or bases). A variety of pharmacologically and pharmaceutically known and accepted salts can be prepared and are envisioned by this invention.

The preferred compositions generally comprise molecules containing a linker, with the molecules having the general structure of: 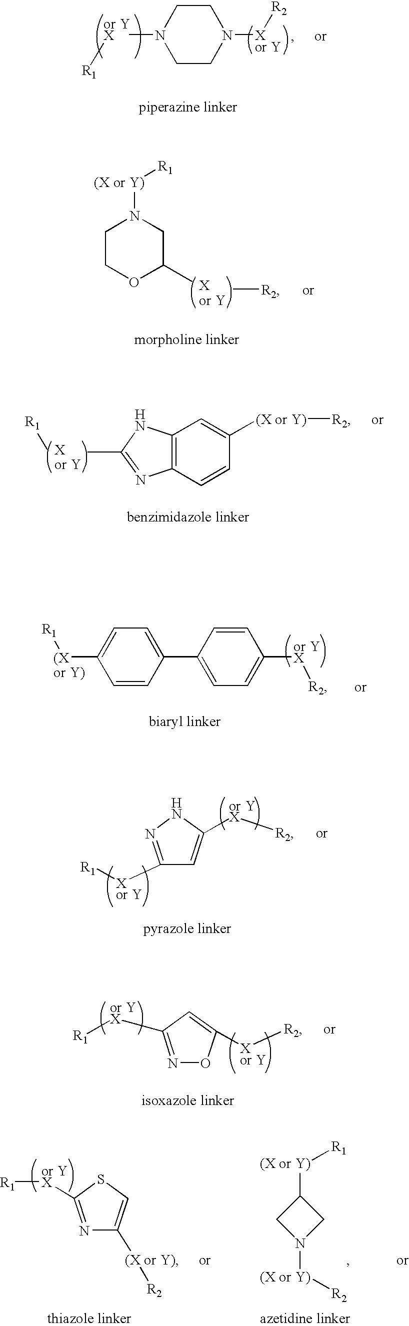 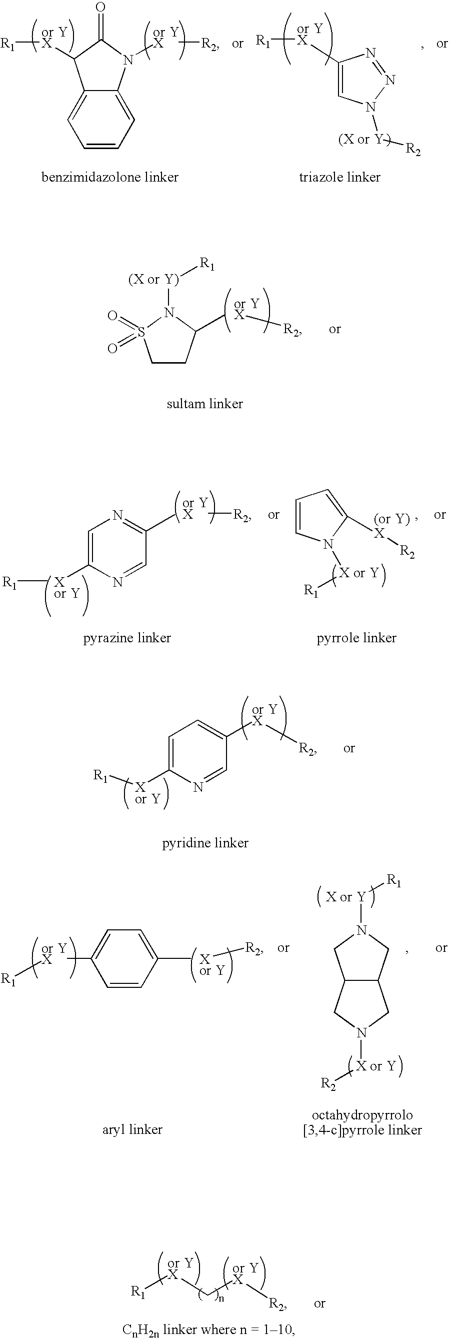 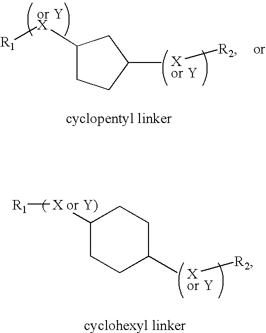
where each of these linkers is drawn with specific positional substitutions, it is recognized that alternate positional isomers are possible (for instance 1,2 or 1,3 or 1,4 substitution on a phenyl ring);
where the coupling group X or Y to a C of the linker can be independently —CₙH₂ₙ (n=1 to 4)-, —O—, —NH—, —CH2NH—, —CH═CH—, —CH₂CH═CH—, —C≡C—, —CH₂C≡C—, —C(═O)—, —CH₂—C(═O)—, —C(═O)O—, —CH₂C(═O)O, —O—C(═O), —C(═O)NH—, —CH₂C(═O)NH—, —NH—C(═O)—, —CH2NH—C(═O)—, —NH—C(═O)—NH—, —CH2NH—C(O)—NH—, —O—C(═O)—NH—, —CH2O—C(═O)—NH—, —CH2NH—C(═O)—O—, or —NH—C(═O)—O—;
where the coupling group X or Y to an N of the linker can be independently —CₙH₂ₙ (n=1 to 4), —CH═CH—, —CH₂CH═CH—, —C≡C—, —CH₂—C≡C—, —C(═O)—, —CH₂—C(═O)—, —C(═O)O—, —CH₂C(═O)O— —C(═O)NH—, or —CH₂C(═O)NH—; and
where R₁ or R₂ can be independently either a hydrophobic or a hydrophilic substituent, where the hydrophobic substituent can be straight or branched or cyclic aliphatic chains of 1-10 carbons, and may be saturated or unsaturated or aromatic; and the hydrophilic substituent can be −2-morpholine, -3-morpholine, -4-morpholine, -2-thiomorpholine, -3-thiomorpholine, -4-thiomorpholine, -2-pyridine, -3-pyridine, -4-pyridine, -2-cyclohexylamine, -3-cyclohexylamine, -4-cyclohexylamine, -2-cyclopentylamine, -3-cyclopentylamine, -2-cyclobutylamine, -3-cyclobutylamine, -2-piperidine, -3-piperidine, -4-piperidine, -2-piperazine, -3-piperazine, -2-pyrrolidine, -3-pyrrolidine, -2-pyrrole, -3-pyrrole, -3-pyrazole, -4-pyrazole, -5-pyrazole, -2-imidazole, -4-imidazole, -5-imidazole, -2-azetidine, or -3-azetidine; or
—CₙH₂ₙ—NR₃R₄ where n=2-8 and R₃ and R₄ are independently hydrogen, methyl, ethyl, propyl or iso-propyl; or
—CₙH₂ₙ—NHC(═NH)NH₂ where n=2-8; or
—C₆H₄—NR₃R₄ where R₃ and R₄ are independently hydrogen, methyl, ethyl, propyl or iso-propyl; or
—CₙH₂ₙ—OH where n=2-8; or
CₙH₂ₙ—COOR₃ where n=2-8 and R₃ is independently hydrogen, methyl, ethyl, propyl or iso-propyl; or
—CₙH₂ₙ—CONR₃R₄ where n=2-8 and R₃ and R₄ are independently hydrogen, methyl, ethyl, propyl, iso-propyl.

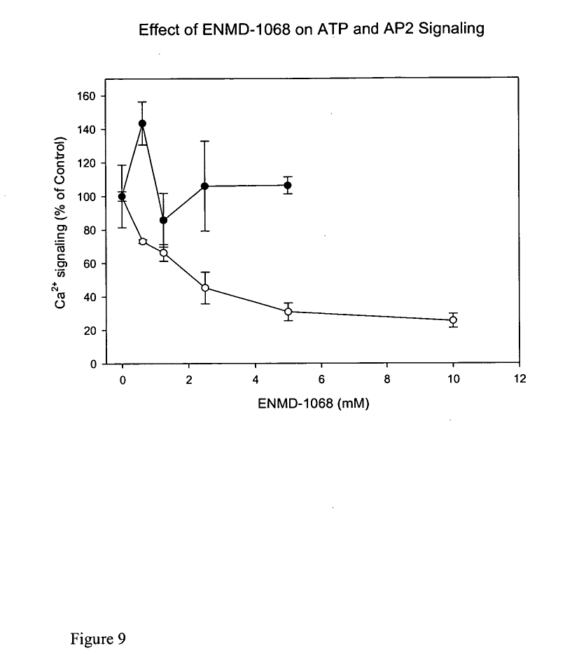

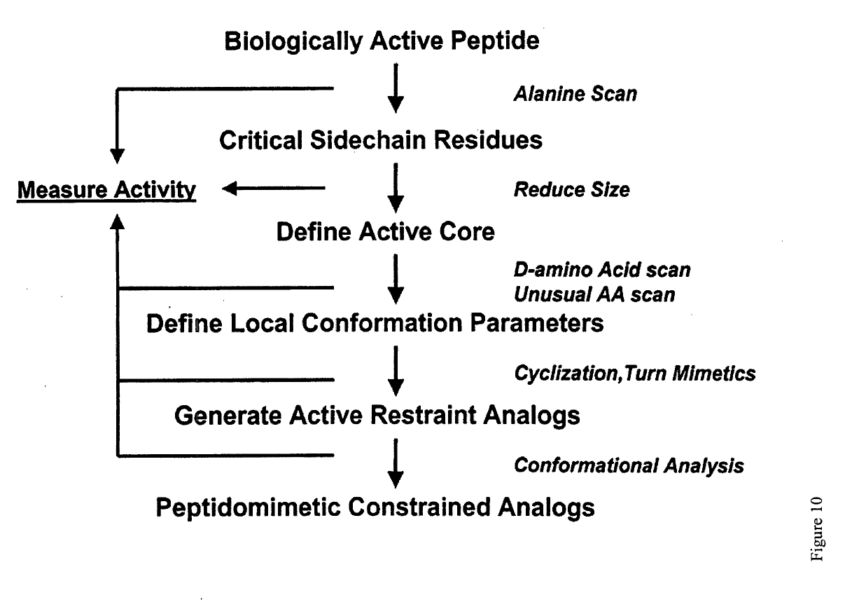

One mimetic of the LIGK (ENMD-1005) (SEQ ID NO:1) antagonist peptide of particular interest is ENMD-1068. The structure of ENMD-1068 comprises a piperazine linker to which a polar 6-amino-hexanoic acid moiety is attached via a heteroatom of the linker, and a hydrophobic isovaleric acid moiety is attached to the other linker heteroatom (Scheme 1). ENMD-1068 was discovered to be an inhibitor of PAR-2 signaling in vitro (FIG. 9). ENMD-1068 has no inhibitory effects on signaling by ATP (FIG. 9). This molecule, due to its enhanced activity, may provide insight into the design and synthesis of other PAR-2 antagonist molecules.

These studies, taken together, demonstrate that PAR-2 plays a very important role in the promotion of angiogenesis and the regulation of inflammation. Furthermore, the inventors demonstrate a way in which activation of coagulation-related pathways may promote tumor growth or angiogenesis through a process that is independent of coagulation. Though not wishing to be bound by the theory, it is possible that the TF/fVIIa complex may be responsible for activating PAR-2 in angiogenic and tumor models. However, several other proteinases can activate PAR-2, and may promote these PAR-2 activities. The present inhibitors inhibit activation of PAR-2 independent of the proteinase which activates it. The most relevant enzymes for these processes are mast cell tryptase, trypsin and matriptase. Thus, the TF/fVIIa—PAR-2 pathway is a very strong candidate for the proangiogenic and protumor activities described here and in earlier applications by these inventors. Specific inhibitors of the TF/fVIIa signaling complex as well as specific inhibitors of the signaling receptor also have antitumor and antiangiogenic activity. Recent studies on TF demonstrate that this molecule is an immediate early gene that is expressed on angiogenic endothelium. Thus, this PAR-2 activator is upregulated and present at the site of angiogenesis. The present studies demonstrating an antiangiogenic activity for LIGK (ENMD-1005) (SEQ ID NO:1), and the predicted antitumor activity this antiangiogenic activity might induce, does not exclude a direct antitumor activity.

In accordance with the methods of the present invention, the compositions described herein, containing a protein, peptide, protein fragment, or molecule including all or an active portion of a ligand that inhibits PARs, optionally in a pharmaceutically acceptable carrier, is administered to a human or animal in an amount sufficient to inhibit undesirable cell proliferation, particularly endothelial cell proliferation, angiogenesis or an angiogenesis-related disease, such as cancer, inflammation, inflammatory processes or inflammatory diseases.

Definitions

The terms “a”, “an” and “the” as used herein are defined to mean one or more and include the plural unless the context is inappropriate.

As used herein, the phrase “proteinase activated receptor” is defined to encompass all proteinase activated receptors (PARs), including, but not limited to, PAR-1, PAR-2, PAR-3 and PAR-4.

The term “antagonist” is used herein to define a protein, peptide or molecule that inhibits proteinase activated receptor activity.

The term “active portion” is defined herein as the portion of a ligand or molecule necessary for inhibiting the activity of proteinase activated receptors. The active portion has the ability to inhibit proteinase activated receptors as determined by in vivo or in vitro assays or other known techniques.

The term “mimetic” is generally defined as a compound that mimics a biological material in its structure or function.

The term peptidomimetic is generally defined as a compound containing non-peptidic structural elements that is capable of mimicking or antagonizing the biological action(s) of a natural parent peptide.

The term “peptides” describes chains of amino acids (typically L-amino acids) whose alpha carbons are linked through peptide bonds formed by a condensation reaction between the carboxyl group of the alpha carbon of one amino acid and the amino group of the alpha carbon of another amino acid. In naturally occurring peptides, in most cases, the terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the peptide, or to the alpha-amino group (amido group when participating in a peptide bond) of an amino acid at any other location within the peptide. Similarly, the term “carboxy terminus” (abbreviated C-terminus) refers to the free carboxyl group on the amino acid at the carboxy terminus of a peptide, or to the carboxyl group of an amino acid at any other location within the peptide.

Typically, the amino acids making up a peptide are numbered in order, starting at the amino terminal and increasing in the direction toward the carboxy terminal of the peptide. Thus, when one amino acid is said to “follow” another, that amino acid is positioned closer to the carboxy terminal of the peptide than the preceding amino acid. Here, naturally occurring amino acids are represented in the text by the commonly used one letter codes (e.g. G=glycine).

The term “residue” is used herein to refer to an amino acid (D or L enantiomer) that is incorporated into a peptide by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

Furthermore, one skilled in the art will recognize that, as mentioned above, individual substitutions, deletions or additions which alter, add or delete a single amino acid or several amino acids in a sequence are conservatively modified variations where the alterations result in the substitution of an amino acid with a chemically similar amino acid. Conservative substitution tables providing functionally similar amino acids are well known in the art. The following six groups each contain examples of amino acids that are frequently considered as conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K); Glutamine (Q);
5) Isoleucine (I), Leucine (L), Methionine (M), Valine (V);
6) Phenylalanine (F), Tyrosine (Y), Tryptophan (W).

Typically, the isolated, antiproliferative peptides described herein are at least about 80% pure, usually at least about 90%, and preferably at least about 95% as measured by HPLC.

When peptides are relatively short in length (i.e., less than about 50 amino acids), they are often synthesized using chemical peptide synthesis techniques. Solid phase synthesis is a method in which the C-terminal amino acid of the sequence is attached to an insoluble support followed by sequential addition of the remaining amino acids in the sequence. This is a preferred method for the chemical synthesis of the peptides described herein. Techniques for solid phase synthesis are known to those skilled in the art.

Short peptides and related amides can also by synthesized efficiently by solution phase coupling chemistry. Amino acids and related molecules, with the appropriate protection groups, are coupled in solution to yield amides and peptides. Coupling reagents for forming amide bonds include, but are not limited to, 1,3-dicyclohexyl carbodiimide, 1-hydroxybenzotriazole and N,N-diisopropylethyl amine or carbonyl diimidizole.

As employed herein, the phrase “biological activity” refers to the functionality, reactivity, and specificity of compounds that are derived from biological systems or those compounds that are reactive to them, or other compounds that mimic the functionality, reactivity, and specificity of these compounds. Examples of suitable biologically active compounds include, but are not limited to, enzymes, antibodies, antigens and proteins.

The term “bodily fluid,” as used herein, includes, but is not limited to, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, and nasal secretions.

The inhibitory proteins and peptides of proteinase activated receptors of the present invention may be isolated from body fluids including, but not limited to, serum, urine, and ascites, or may be synthesized by chemical or biological methods, such as cell culture, recombinant gene expression, and peptide synthesis. Recombinant techniques include gene amplification from DNA sources using the polymerase chain reaction (PCR), and gene amplification from RNA sources using reverse transcriptase/PCR. Ligands of interest can be extracted from body fluids by known protein extraction methods, particularly the method described by Novotny, W. F., et al., J. Biol. Chem. 264:18832-18837 (1989).

Peptides or Protein Fragments

Peptides or protein fragments comprising PAR antagonists can be produced as described above and tested for inhibitory activity using techniques and methods known to those skilled in the art. Full length proteins can be cleaved into individual domains or digested using various methods such as, for example, the method described by Enjyoji et al. (Biochemistry 34:5725-5735 (1995)), which is incorporated herein by reference in its entirety.

Alternatively, fragments are prepared by digesting the entire protein, or large fragments thereof exhibiting anti-proliferative activity, to remove one amino acid at a time. Each progressively shorter fragment is then tested for anti-proliferative activity. Similarly, fragments of various lengths may be synthesized and tested for inhibitory activity. By increasing or decreasing the length of a fragment, one skilled in the art may determine the exact number, identity, and sequence of amino acids within the protein that are required for inhibitory activity using routine digestion, synthesis, and screening procedures known to those skilled in the art.

Inhibitory activity is evaluated in situ by testing the ability of the proteins, molecules and peptides to inhibit the activation of PAR. Suitable assays are well known to skilled in the art and several examples of such are provided below in the Examples. Antiangiogenic activity may be assessed using the mouse Matrigel plug assay, described by Kibbey, M. C. et al. (1992) J. Natl. Cancer Inst. 84, 1633-8, which is incorporated herein by reference in its entirety. The Matrigel assay is briefly described as follows. Groups of 10 animals were injected with 0.5 ml of Matrigel (Collaborative Research) to which FGF-2 (final concentration 2 ug/ml) was added. This mixture was then injected subcutaneously at the ventral midline, posterior to the xiphiod process. Animals were treated daily with compound or control buffer intraperitoneally. After 6 days, animals were euthanized with CO₂. The Matrigel plug was removed, and weighed, then 1 ml of water was added to the plug and frozen. Angiogenesis was quantified by measuring hemoglobin within the plug. First, the plug was homogenized, and centrifuged at 20,000 g for 20 minutes. The supernatant was retained and the amount of hemoglobin was quantified using the Sigma hemoglobin kit (527-A). Control animals were injected with Matrigel lacking bFGF. Another suitable assay is the HUVEC proliferation assay.

Also included in the present invention are peptides having conservatively modified variations in comparison to the claimed peptides, wherein the activity of the peptide is not significantly different from that of the claimed peptide.

Formulations

The naturally occurring or synthetic protein, molecule, peptide, or protein fragment, containing all or an active portion of a protein, peptide or molecule that may bind to a proteinase activated receptor can be prepared in a physiologically acceptable formulation, such as in a pharmaceutically acceptable carrier, using known techniques. For example, the protein, peptide, protein fragment or non-peptide molecule is combined with a pharmaceutically acceptable excipient to form a therapeutic composition.

Alternatively, the gene for the protein, peptide, or protein fragment, containing all or an active portion of a desired ligand, may be delivered in a vector for continuous administration using gene therapy techniques. The vector may be administered in a vehicle having specificity for a target site, such as a tumor.

The composition may be in the form of a solid, liquid or aerosol. Examples of solid compositions include pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously. Examples of liquid compositions include formulations adapted for injection subcutaneously, intravenously, intra-arterially, and formulations for topical and intraocular administration. Examples of aerosol formulations include inhaler formulations for administration to the lungs. Also envisioned are other compositions for administration including, but not limited to, suppositiories, transdermal, transbuccal, and ocular administration.

The composition may be administered by standard routes of administration. In general, the composition may be administered by topical, oral, rectal, nasal or parenteral (for example, intravenous, subcutaneous, or intermuscular) routes. In addition, the composition may be incorporated into sustained release matrices such as biodegradable polymers, the polymers being implanted in the vicinity of where delivery is desired, for example, at the site of a tumor or site of inflammation. The method includes administration of a single dose, administration of repeated doses at predetermined time intervals, and sustained administration for a predetermined period of time. Examples of biodegradable polymers and their use are described in detail in the January 2005 issue of Molecules, Volume 10, pages 1-180, which is incorporated herein by reference in its entirety.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials including, but not limited to, liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone.

The dosage of the composition will depend on the condition being treated, the particular composition used, and other clinical factors such as weight and condition of the patient, and the route of administration.

Further, the term “effective amount” refers to the amount of the composition which, when administered to a human or animal, inhibits proteinase activated receptor activity, particularly undesirable cell proliferation, causing a reduction in cancer or inhibition in the spread and proliferation of cancer or reduction of an inflammatory condition. The effective amount is readily determined by one of skill in the art following routine procedures.

For example, compositions of the present invention may be administered parenterally or orally in a range of approximately 1.0 μg to 1.0 g per dose, though this range is not intended to be limiting. The actual amount of composition required to elicit an appropriate response will vary for each individual patient depending on the potency of the composition administered and on the response of the individual. Consequently, the specific amount administered to an individual will be determined by routine experimentation and based upon the training and experience of one skilled in the art.

The composition may be administered in combination with other compositions and procedures for the treatment of diseases. For example, unwanted cell proliferation may be treated conventionally with surgery, radiation or chemotherapy in combination with the administration of the composition, and additional doses of the composition may be subsequently administered to the patient to stabilize and inhibit the growth of any residual unwanted cell proliferation.

Antibodies for Proteinase Activated Receptors The present invention further comprises antibodies of PAR antagonists that may be used for diagnostic as well as therapeutic purposes. The antibodies provided herein are monoclonal or polyclonal antibodies having binding specificity for desired ligands. The preferred antibodies are monoclonal antibodies, due to their higher specificity for the ligands. The preferred antibodies will exhibit minimal or no crossreactivity with other proteins or peptides. Preferably, the antibodies are specific for proteinase activated receptor ligands such as AP2 or the agonist sequence of the PAR proteins or for the ligand binding domains of the PAR protein, including, but not limited to, the extracellular loops of any PAR.

Monoclonal antibodies are prepared by immunizing an animal, such as a mouse or rabbit, with a whole or immunogenic portion of a desired peptide, such as SLIGKV (ENMD-1003) (SEQ ID NO:52) or a sequence from the ligand binding site of the PAR ligand, including, but not limited to, the extracellular loops. Spleen cells are harvested from the immunized animals and hybridomas generated by fusing sensitized spleen cells with a myeloma cell line, such as murine SP2/O myeloma cells (ATCC, Manassas, Va.). The cells are induced to fuse by the addition of polyethylene glycol. Hybridomas are chemically selected by plating the cells in a selection medium containing hypoxanthine, aminopterin and thymidine (HAT).

Hybridomas are subsequently screened for the ability to produce monoclonal antibodies against ligands. Hybridomas producing antibodies that bind to the ligands are cloned, expanded and stored frozen for future production. The preferred hybridoma produces a monoclonal antibody having the IgG isotype, more preferably the IgG1 isotype.

The polyclonal antibodies are prepared by immunizing animals, such as mice or rabbits, with a ligand such as antithrombin as described above. Blood sera is subsequently collected from the animals, and antibodies in the sera screened for binding reactivity against the ligand, preferably the antigens that are reactive with the monoclonal antibody described above.

Either the monoclonal antibodies or the polyclonal antibodies, or both may be labeled directly with a detectable label for identification and quantitation of ligands in a biological as described below. Labels for use in immunoassays are generally known to those skilled in the art and include enzymes, radioisotopes, and fluorescent, luminescent and chromogenic substances including colored particles, such as colloidal gold and latex beads. The antibodies may also be bound to a solid phase to facilitate separation of antibody-antigen complexes from non-reacted components in an immunoassay. Exemplary solid phase substances include, but are not limited to, microtiter plates, test tubes, magnetic, plastic or glass beads and slides. Methods for coupling antibodies to solid phases are well known to those skilled in the art.

Alternatively, the antibodies may be labeled indirectly by reaction with labeled substances that have an affinity for immunoglobulin, such as protein A or G or second antibodies. The antibodies may be conjugated with a second substance and detected with a labeled third substance having an affinity for the second substance conjugated to the antibody. For example, the antibodies may be conjugated to biotin and the antibody-biotin conjugate detected using labeled avidin or streptavidin. Similarly, the antibodies may be conjugated to a hapten and the antibody-hapten conjugate detected using labeled anti-hapten antibody. These and other methods of labeling antibodies and assay conjugates are well known to those skilled in the art.

Sensitive immunoassays employing one or more of the antibodies described above are provided by the present invention. The immunoassays are useful for detecting the presence or amount of ligands in a variety of samples, particularly biological samples, such as human or animal biological fluids. The samples may be obtained from any source in which the ligands may exist. For example, the sample may include, but is not limited to, blood, saliva, semen, tears, and urine.

The antibody-antigen complexes formed in the immunoassays of the present invention are detected using immunoassay methods known to those skilled in the art, including sandwich immunoassays and competitive immunoassays. The antibody-antigen complexes are exposed to antibodies similar to those used to capture the antigen, but which have been labeled with a detectable label. Suitable labels include, but are not limited to: chemiluminescent labels, such as horseradish peroxidase; electrochemiluminescent labels, such as ruthenium and aequorin; bioluminescent labels, such as luciferase; fluorescent labels such as FITC; and enzymatic labels such as alkaline phosphatase, β-galactosidase, and horseradish peroxidase.

The labeled complex is then detected using a detection technique or instrument specific for detection of the label employed. Soluble antigen or antigens may also be incubated with magnetic beads coated with non-specific antibodies in an identical assay format to determine the background values of samples analyzed in the assay.

Diseases and Conditions To Be Treated

The methods and compositions described herein are useful for treating human and animal diseases and processes mediated by abnormal or undesirable cellular proliferation, particularly abnormal or undesirable endothelial cell proliferation, including, but not limited to, hemangioma, solid tumors, leukemia, tumor metastasis, telangiectasia, psoriasis scleroderma, pyogenic granuloma, myocardial angiogenesis, plaque neovascularization, coronary collaterals, atherosclerosis, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, fractures, keloids, vasculogenesis, hematopoiesis, endometriosis, ovulation, menstruation, and placentation. The methods and compositions are particularly useful for treating angiogenesis-related disorders and diseases by inhibiting angiogenesis and inflammation.

The methods and compositions described herein are particularly useful for treating cancer, arthritis, macular degeneration, and diabetic retinopathy. Administration of the compositions to a human or animal having prevascularized metastasized tumors is useful for preventing the growth or expansion of such tumors and metastases.

The methods and compositions of this invention are useful for treating the following diseases and conditions and the symptoms associated with the following diseases and conditions: abnormal growth by endothelial cells, acne rosacea, acoustic neuroma, adhesions, angiofibroma, arteriovenous malformations, artery occlusion, arthritis, asthma, capillary proliferation within plaques, atherosclerotic plaques, atopic keratitis, bacterial ulcers, bartonelosis, benign tumors (such as hemangiomas, acoustic neuromas, neurofibromas, trachomas, pyogenic granulomas), benign, premalignant and malignant vulvar lesions, Best's disease, bladder cancers, block implantation of a blastula, block menstruation (induce amenorrhea), block ovulation, blood-borne tumors (including leukemias, and neoplastic diseases of the bone marrow), bone marrow abnormalities including any of various acute or chronic neoplastic diseases of the bone marrow in which unrestrained proliferation of white blood cells occurs including multiple myeloma, bone growth and repair, breast cancer, burns, hypertrophy following cancer (including solid tumors: rhabdomyosarcomas, retinoblastoma, Ewing's sarcoma, neuroblastoma, osteosarcoma, blood-borne tumors, leukemias, neoplastic diseases of the bone marrow, multiple myeloma diseases and hemangiomas), carotid obstructive disease, central nervous system malignancy, certain immune reactions (for example immune disorders/reactions), cervical cancers, chemical burns, cholesteatoma especially of the middle ear, choroidal neovascularization, choroiditis, chronic or acute inflammation, chronically exercised muscle, cirrhotic liver, contact lens overwear, corneal diseases, corneal graft neovasularization, corneal graft rejection, corneal neovascularization diseases (including, but not limited to, epidemic keratoconjunctivitis, Vitamin A deficiency, contact lens overwear, atopic keratitis, superior limbic keratitis, and pterygium keratitis sicca), corpus luteum formation, Crohn's disease, delayed wound healing, diabetes, diabetic (proliferative) retinopathy, diseases caused by the abnormal proliferation of fibrovascular or fibrous tissue including all forms of prolific vitreoretinopathy (PVR), Eales disease, empyema of the thorax, endometriosis, endometrium, epidemic keratoconjuctivitis, excessive or abnormal stimulation of endothelial cells (such as atherosclerosis), eye-related diseases (including rubeosis (neovascularization of the angle), female reproductive system conditions (including neovascularization of ovarian follicles, corpus luteum, maternal decidua, repair of endometrial vessels, angiogenesis in embryonic implantation sites (ovarian hyperstimulation syndromes), embryonic development, folliculogenesis, luteogenesis, normal menstruating endometrium), fibrinolysis, fibroplasias (retrolental and excessive repair in wound healing), fibrosing alveolitis, fungal ulcers, gastrointestinal infections, peptic ulcer, ulcerative colitis, inflammed polyps, intestinal graft-vs-host reaction, neoplastic tumors, mastocytosis, intestinal ischemia, neovascular glaucoma, gout or gouty arthritis, graft versus host rejection (also chronic and acute rejection), granulation tissue of healing wounds, burn granulations, haemangiomatoses (systemic forms of hemangiomas), hand foot and mouth disease, hair growth, hemangioma, hemophiliac joints, hereditary diseases (including Osler-Weber-Rendu disease), Herpes simplex, Herpes zoster, HHT (hereditary hemorrhagic telangiectasia), hypertrophic scars, hypertrophy following surgery, burns and injury, hyperviscosity syndromes, immune disorders, immune reactions, implantation of embryo (2-8 weeks), infections causing retinitis, infectious diseases caused by microorganisms, inflammation, inflammatory disorders, immune and non-immune inflammatory reactions, inflammed joints, Kaposi's sarcoma, leprosy, leukemias, lipid degeneration (lipid keratopathy), lipoma, lung cancer, lupus (lupus erythematosis, systemic lupus erythematosis), Lyme disease, age-related macular degeneration (subretinal neovascularization), marginal keratolysis, melanoma, meningiomas, mesothelioma, metastasis of tumors, Mooren's ulcer, mycobacteria diseases, myeloma, multiple myeloma diseases, myopia, neoplasias, neoplastic diseases of the bone marrow (any of various acute or chronic diseases in which unrestrained proliferation of white blood cells occurs which are blood-borne tumors, including leukemias), neovascular glaucoma, neovascularization of the angle, neuroblastoma, neurofibroma, neurofibromatosis, neurofibrosarcoma, non-union fractures, ocular angiogenic diseases (including diabetic retinopathy, retinopathy of prematurity, and retrolental fibroplasia, macular degeneration, corneal graft rejection, neovascular glaucoma, and Osler Weber syndrome (Osler-Weber-Rendu disease), ocular histoplasmosis, ocular neovascular disease, ocular tumors, optic pits, oral cancers, osteoarthritis, osteomyelitis, osteosarcoma, Paget's disease (osteitis deformans), parasitic diseases, pars planitis, pemphigoid, phlyctenulosis, polyarteritis, post-laser complications, proliferation of white blood cells (such as any of various acute or chronic neoplastic diseases of the bone marrow, in which unrestrained proliferation of white blood cells occurs), prostate cancer, protozoan infections, pseudoxanthoma elasticum, psoriasis, pterygium (keratitis sicca), pulmonary fibrosis, pyogenic granuloma, radial keratotomy, chronic and acute rejection, retinal detachment, retinitis, retinoblastoma, retinopathy of prematurity, retrolental fibroplasia, rhabdomyosarcomas, rheumatoid arthritis, rheumatoid synovial hypertrophy (arthritis), rosacea (acne rosacea), rubeosis, sarcoidosis, scleritis, scleroderma, sicca (including pterygium (keratitis sicca) and Sjogren's (sicca) syndrome), sickle cell anemia, skin disease (including melanoma, pyogenic granulomas, psoriasis, hemangioma, skin warts, and HPV type 2 (human papillomavirus)), solid tumors (including rhabdomyosarcomas, retinoblastoma, neuroblastoma, osteosarcoma), Stargard's disease, Stevens-Johnson's disease, superior limbic keratitis (superior limbic keratoconjuctivitis, SLK), hypertrophic scars, wound granulation and vascular adhesions, syphilis, systemic lupus, systemic lupus erythematosis, Terrien's marginal degeneration, toxoplasmosis, trachoma, trauma, tuberculosis, ulcerative colitis, ulcers (including fungal, Mooren's, peptic and bacterial), undesired angiogenesis in normal processes (including wound healing, female reproductive functions, bone repair, hair growth, chronic uveitis, and vascular malfunction), vascular tumors, vein occlusion, vitamin A deficiency, chronic vitritis, Wegener's sarcoidosis, white blood cells diseases (including any acute or chronic neoplastic diseases of the bone marrow in which unrestrained proliferation of white blood cells occurs), wound healing and inappropriate wound healing, delayed wound healing (for instance in angiofibroma, arteriovenous malformations, arthritis, atherosclerotic plaques, corneal graft neovascularization, diabetic retinopathy, hemangioma, hemophilic joints, hypertrophic scars, neovascular glaucoma, non-union fractures, pyogenic granuloma, retrolental fibroplasias, scleroderma, solid tumors, trachoma, corpus luteum formations, chronically exercised muscle, rheumatoid arthritis, solid tumors, and chronic inflammatory diseases, inflamed joints, rheumatoid synovial hypertrophy (arthritis), metastasis, oral cancers, cervical cancers, bladder and breast cancers, melanomas, pyogenic granulomas, haemangiomatoses, Kaposi's sarcoma, adhesions, acute and/or chronic inflammation and inflammatory reactions, and chronic and acute rejection).

In addition, the methods and compositions of this invention are also useful for treating the following diseases and the symptoms associated with asthma, bronchogenic carcinoma, sarcoidosis, ankylosing spondylosis, chronic obstructive pulmonary disease, thyroiditis (including subacute, acute and chronic thyroiditis, granulomatous (or DeQuervain's thyroiditis) lymphocytic thyroiditis (Hashimoto's thyroiditis), invasive fibrous (Riedel's) thyroiditis, pyogenic or suppurative thyroiditis), dermatitis (including psoriasis, eczema, dermatitis, seborrheic dermatitis, contact dermatitis, atopic dermatitis, nummular dermatitis, chronic dermatitis, lichen simplex chronicus, stasis dermatitis, generalized exfoliative dermatitis and Behçet's Syndrome), adenomatous polyposis coli, Alagille syndrome, appendicitis, Barrett esophagus, biliary atresia, biliary tract diseases, Caroli disease, celiac disease, cholangitis, cholecystitis, cholelithiasis, ulcerative colitis, Crohn's disease, digestive system diseases, duodenal ulcer, dysentery, pseudomembranous enterocolitis, esophageal achalasia, esophageal atresia, esophagitis, fatty liver, gastritis, hypertrophic gastritis, gastroenteritis, gastroesophageal reflux, gastroparesis, hepatitis, chronic hepatitis, Hirschsprung disease, inflammatory bowel diseases, intestinal neoplasms, intestinal neuronal dysplasia, liver cirrhosis, Meckel diverticulum, pancreatic diseases (including pancreatic insufficiency, pancreatic neoplasms, and pancreatitis), peptic ulcer, Peutz-Jeghers syndrome, proctitis, Whipple disease, Zollinger-Ellison syndrome, multiple sclerosis, neuritis, Alzheimer's disease and other neurological diseases, bronchiolitis obliterans organising pneumonia, bronchiectasis, pulmonary fibrosis, chronic obstructive pulmonary syndrome, systemic sclerosis, pleural inflammation, seronegative spondylarthropathies, septic arthritis, prolonged pulmonary eosinophilia, simple pulmonary eosinophilia, Löffler's syndrome, pulmonary eosinophilia with asthma, polyarteritis nodosa, chronic eosinophilic pneumonia, acute eosinophilic pneumonia, idiopathic hypereosinophilic syndrome, allergic bronchopulmonary aspergillosis, bronchocentric granulomatosis, allergic angiitis and granulomatosis (Churg-Strauss Syndrome), idiopathic pulmonary fibrosis, Langerhan's cell granulomatosis (Eosinophilic Granuloma), chronic bronchitis, emphysema, interstitial pneumonia, cutaneous mastocytoma, urticaria pigmentosa, telangiectasia macularis eruptiva perstans (TMEP), systemic mast cell disease, mast cell leukemia, eosinophilic fasciitis, eosinophilic gastroenteritis, eosinophilia myalgia syndrome, systemic mastocytosis, mastocytosis, reactive mastocytosis, neuritis, vestibular neuritis, optic neuritis, lupus nephritis, nephritis, and Parkinson's diseases.

The compositions and methods are further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

The following experiments were conducted using methods and protocols well known to those skilled in the art. Details regarding the procedures used are found throughout the scientific literature and also for example in United States patents.

### EXAMPLES

### Example 1

### PAR Signalling Activity

Confluent HUVECs, Lewis lung carcinoma cells or U87-MG glioma cells or HT29 colon carcinoma cells were loaded for 30-60 minutes with the fluorescent dye Fluo-4. Final concentration 4 uM Fluo-4, 0.02% pluronic acid in physiological buffer. Cells were then washed with assay buffer, (HBSS containing 1 mM CaCl₂, 1 mM MgSO₄, and 2.5 mM probenecid). Cells were stimulated with various doses of PAR-2 activating peptide, PAR-1 activating peptide or ATP. Fluorescence was monitored using a Wallac 1470 fluorescent plate reader. (See Al-ani et. al Journal of Pharmacology and Experimental Therapeutics 290:2, 753-760)

Calcium mobilization curves of the PAR-2 agonist SLIGKV (ENMD-1003) (SEQ ID NO:52) compared with two truncated molecules LIGK (ENMD-1005) (SEQ ID NO:1) and LIGKV (ENMD-1007) (SEQ ID NO:2) are provided in FIG. 2A. Neither truncated molecule was able to induce calcium mobilization, in contrast with SLIGKV (ENMD-1003) (SEQ ID NO:52), which demonstrates the typical spike of calcium release followed by degradation of signal. Similar studies were performed on alanine substituted SLIGKV (ENMD-1003) (SEQ ID NO:52) peptides (FIGS. 2B and 2C). It was found that substitution of SLIGKV (ENMD-1003) (SEQ ID NO:52) at S, L, I, or K abrogated or significantly diminished signaling activity, while two substituted peptides, SLIAKV (ENMD-1011) (SEQ ID NO:54) and SLIGKA (ENMD-1013) (SEQ ID NO:55) demonstrated robust signaling activity.

TABLE 1

| | | ENMD ID | Activate | Inhibit |
|---|---|---|---|---|
| Peptide | SEQ ID | NO: | PAR-2 | PAR-2 |
| SLIGKV | SEQ ID NO: 52 | ENMD-1003 | ++++ | NA |
| SLIGK | SEQ ID NO: 57 | ENMD-1006 | ++ | NA |
| LIGKV | SEQ ID NO: 2 | ENMD-1007 | − | + |
| LIGK | SEQ ID NO: 1 | ENMD-1005 | − | ++++ |
| ALIGKV | SEQ ID NO: 58 | ENMD-1008 | − | − |
| SAIGKV | SEQ ID NO: 59 | ENMD-1009 | − | − |
| SLAGKV | SEQ ID NO: 60 | ENMD-1010 | − | − |
| SLIAKV | SEQ ID NO: 54 | ENMD-1011 | ++ | − |
| SLIGAV | SEQ ID NO: 61 | ENMD-1012 | +/− | − |
| SLIGKA | SEQ ID NO: 55 | ENMD-1013 | ++ | − |

### Example 2

### Identification and Testing of PAR-2 Antagonists

In order to assess the potential of peptides and molecules selected above to block PAR-2 signaling, cells were pretreated with potential antagonist peptides for a predetermined amount of time and were subsequently treated with various GPCR agonists. Confluent Lewis lung carcinoma (LLC) cells were loaded for 30-60 minutes with the fluorescent dye Fluo-4. Final concentration 4 uM Fluo-4, 0.02% pluronic acid in physiological buffer. Cells were then washed with assay buffer, (HBSS containing 1 mM CaCl₂, 1 mM MgSO₄, and 2.5 mM probenecid). Cells were stimulated with various doses of PAR-2 activating peptide, PAR-1 activating peptide or ATP. Fluorescence was monitored using a Wallac 1470 fluorescent plate reader. (See Al-ani et. al Journal of Pharmacology and Experimental Therapeutics 290:2, 753-760). Additional compound screening was performed using U87-MG human glioma cells. In this assay cells were labeled with FLIPR Calcium 3 dye (Component A), which was dissolved in 10 mls of Assay Buffer without Probenecid. Loading buffer was prepared by diluting Component A with an additional 90 mls of Assay Buffer without Probenecid, giving a total volume of a 100 ml. For plate loading, 11 mls of FLIPR Calcium 3 dye (Component A) is placed into a 15 ml conical tube+110 μl of 250 mM Probenecid, at a final in-well working volume of 2.5 mM. Finally, 50 μl of Calcium 3 dye was added wells containing 100 μl media, and incubated 1 hr, 37° C., 5% CO₂. Calcium signaling was then measured using a Flexstation II (Molecular Devices) following manufacturer's instructions.

Potential antagonist compounds were initially screened at 1 mM compound concentration. Those compounds with activity were then subjected to additional confirmatory testing. Activity is reported as treated/controlled (T/C) where the area under the curve (AUC) of calcium mobilization plots in treated wells is divided by the AUC of calcium mobilization plots in control wells to generate a measure of inhibition.

TABLE 2

| | | | PAR-2 AP2 |
|---|---|---|---|
| | | | Signaling |
| | | ENMD ID | Treated/Control |
| Peptide Sequence | SEQ ID NO: | number | (T/C) |
| LIGK-amide | SEQ ID NO: 62 | ENMD-1020 | 0.54 |
| IGK-amide | SEQ ID NO: 31 | ENMD-1021 | 0.67 |
| LIGR | SEQ ID NO: 32 | ENMD-1022 | 0.26 |
| IGR | SEQ ID NO: 12 | ENMD-1023 | 0.23 |
| IG-Dab | SEQ ID NO: 15 | ENMD-1024 | 0.27 |
| IG-Dap | SEQ ID NO: 16 | ENMD-1025 | 0.31 |
| LIG-Dab | SEQ ID NO: 17 | ENMD-1026 | 0.23 |
| LIG-Dap | SEQ ID NO: 19 | ENMD-1027 | 0.30 |
| LIG-Orn | SEQ ID NO: 21 | ENMD-1028 | 0.36 |
| IG-Orn | SEQ ID NO: 24 | ENMD-1029 | 0.42 |
| LIG-(4-aminoF) | SEQ ID NO: 25 | ENMD-1030 | 0.05 |
| dL-dI-dG-dK | SEQ ID NO: 27 | ENMD-1032 | 0.44 |
| dI-dG-dK | SEQ ID NO: 28 | ENMD-1383 | 0.48 |
| LIG-dK | SEQ ID NO: 29 | ENMD-1384 | 0.16 |
| IG-dK | SEQ ID NO: 30 | ENMD-1087 | 0.33 |
| LIGD | SEQ ID NO: 33 | ENMD-1045 | 0.39 |
| LIGE | SEQ ID NO: 34 | ENMD-1046 | 0.34 |
| LIGN | SEQ ID NO: 35 | ENMD-1047 | 0.88 |
| LIGQ | SEQ ID NO: 36 | ENMD-1048 | 0.59 |
| LIGS | SEQ ID NO: 37 | ENMD-1049 | 0.64 |
| LIGT | SEQ ID NO: 38 | ENMD-1050 | 0.70 |
| LIGY | SEQ ID NO: 39 | ENMD-1051 | 0.83 |
| LIPK | SEQ ID NO: 40 | ENMD-1052 | 0.89 |
| LPGK | SEQ ID NO: 41 | ENMD-1053 | 0.89 |
| LIGH | SEQ ID NO: 42 | ENMD-1054 | 0.83 |
| L-Sta-K | SEQ ID NO: 43 | ENMD-1056 | 1.35 |
| L-Sta-GK | SEQ ID NO: 44 | ENMD-1057 | 1.40 |
| L-NiP-K | SEQ ID NO: 45 | ENMD-1058 | 1.58 |
| L-Nip-GK | SEQ ID NO: 46 | ENMD-1059 | 0.97 |
| L-HyP-K | SEQ ID NO: 47 | ENMD-1060 | 1.29 |
| L-HyP-GK | SEQ ID NO: 48 | ENMD-1061 | 1.16 |
| L-Imid-K | SEQ ID NO: 49 | ENMD-1062 | 0.94 |
| L-Imid-GK | SEQ ID NO: 50 | ENMD-1063 | 0.94 |
| LIGM | SEQ ID NO: 51 | ENMD-1064 | 1.19 |

TABLE 3
Table of PAR-2 Mimetic Antagonists

| | PAR-2 AP2 | | PAR-2 AP2 |
|---|---|---|---|
| PAR2 | Signaling | PAR2 | Signaling |
| Antagonist | Treated/Control | Antagonist | Treated/Control |
| 1033 | 0.28 | 1529 | 0.35 |
| 1034 | 0.39 | 1533 | 0.68 |
| 1035 | 0.32 | 1534 | 0.44 |
| 1036 | 0.55 | 1535 | 0.46 |
| 1037 | 0.55 | 1536 | 0.42 |
| 1038 | 0.35 | 1537 | 0.91 |
| 1039 | 1.34 | 1538 | 0.48 |
| 1040 | 1.04 | 1539 | 0.25 |
| 1041 | 0.17 | 1540 | 0.05 |
| 1065 | 0.34 | 1541 | 0.65 |
| 1066 | 0.20 | 1543 | 0.55 |
| 1067 | 0.77 | 1545 | 0.35 |
| 1068 | 0.58 | 1546 | 0.97 |
| 1069 | 0.94 | 1547 | 0.24 |
| 1070 | 0.05 | 1549 | 0.26 |
| 1071 | 0.16 | 1550 | 0.63 |
| 1072 | 0.56 | 1551 | 0.66 |
| 1073 | 0.56 | 1552 | 0.10 |
| 1074 | 0.35 | 1553 | 0.81 |
| 1075 | 0.71 | 1554 | 0.44 |
| 1076 | 0.14 | 1555 | 0.86 |
| 1077 | 0.80 | 1556 | 0.57 |
| 1078 | 0.42 | 1557 | 0.52 |
| 1079 | 0.90 | 1558 | 0.60 |
| 1391 | 0.01 | 1559 | 0.52 |
| 1393 | 0.81 | 1560 | 0.48 |
| 1397 | 0.15 | 1561 | 0.89 |
| 1402 | 0.64 | 1562 | 0.57 |
| 1405 | 0.13 | 1563 | 0.62 |
| 1406 | 0.24 | 1564 | 1.16 |
| 1408 | 0.35 | 1566 | 0.01 |
| 1409 | 0.45 | 1567 | 0.08 |
| 1410 | 0.55 | 1568 | 0.92 |
| 1411 | 0.43 | 1569 | 0.77 |
| 1416 | 0.48 | 1570 | 0.05 |
| 1417 | 0.38 | 1571 | 0.81 |
| 1418 | 0.49 | 1572 | 0.10 |
| 1504 | 0.71 | 1573 | 0.01 |
| 1505 | 0.19 | 1574 | 0.01 |
| 1509 | 0.71 | 1763 | 0.83 |
| 1511 | 0.13 | 1764 | 0.96 |
| 1513 | 0.97 | 1766 | 0.73 |
| 1514 | 0.53 | 1768 | 0.10 |
| 1515 | 0.65 | 1770 | 0.91 |
| 1516 | 0.87 | 1771 | 0.05 |
| 1517 | 0.52 | 1772 | 0.73 |
| 1518 | 0.58 | 1773 | 0.85 |
| 1519 | 0.65 | 1774 | 0.75 |
| 1520 | 0.36 | 1775 | 0.90 |
| 1521 | 1.01 | 1776 | 0.82 |
| 1522 | 1.00 | 1777 | 0.64 |
| 1523 | 0.54 | 1778 | 0.05 |
| 1524 | 0.73 | 1779 | 0.89 |
| 1525 | 0.52 | 1780 | 0.86 |
| 1526 | 0.64 | 1781 | 0.71 |
| 1527 | 0.64 | | |

### Example 3

### Activation Study for Assessing Inhibitory Activity of LIGK (ENMD-1005) (SEQ ID NO:1) using ATP and SFLLRN (ENMD-1014) (SEQ ID NO:56)

In order to demonstrate that LIGK (ENMD-1005) (SEQ ID NO:1) is a specific inhibitor of PAR-2 signaling, activation studies were performed with ATP and the PAR-1 activation peptide, SFLLRN (ENMD-1014) (SEQ ID NO:56), on cells that were pretreated with LIGK (ENMD-1005) (SEQ ID NO:1). Both of these molecules signal through GPCRs, and PAR-1 is highly homologous to PAR-2, to the degree that the PAR-1 agonist peptide can signal through PAR-2 at high concentrations. In both cases, the PAR-2 antagonist LIGK (ENMD-1005) (SEQ ID NO:1) had no inhibitory effect on signaling (FIG. 5).

### Example 4

### In Vivo Analysis of LIGK (ENMD-1005) (SEQ ID NO:1) Inhibitory Effect on PAR-2

C57BL6 mice were injected with 5-25 μg of SLIGKV (ENMD-1003) (SEQ ID NO:52) into their footpad, in the presence or absence of increasing amounts of various PAR-2 antagonists. One hour later, footpad (tarsus) thickness was measured to quantify inflammation (edema).

The inventors next assessed whether the LIGK (ENMD-1005) (SEQ ID NO:1) peptide had in vivo PAR-2 antagonistic activity. This was studied using an edema model where vascular permeability was induced by the PAR-2 agonist peptide. In this model, the PAR-2 peptide induces severe edema as previously reported (FIG. 6). This vascular response was blocked by co-treatment with the PAR-2 antagonist LIGK (ENMD-1005) (SEQ ID NO:1) (FIG. 6). Thus, LIGK (ENMD-1005) (SEQ ID NO:1) functions in vivo to block PAR-2 signaling.

### Example 5

### Inhibitory Activity of LIGK (ENMD-1005) (SEQ ID NO:1) in In Vivo Matrigel Angiogenesis Assay

C57/BL6 mice were injected subcutaneously with Matrigel containing 0.5 μg FGF-2. Treatment was started at day 1 with LIGK (ENMD-1005) (SEQ ID NO:1) administered subcutaneously daily for 6 days.

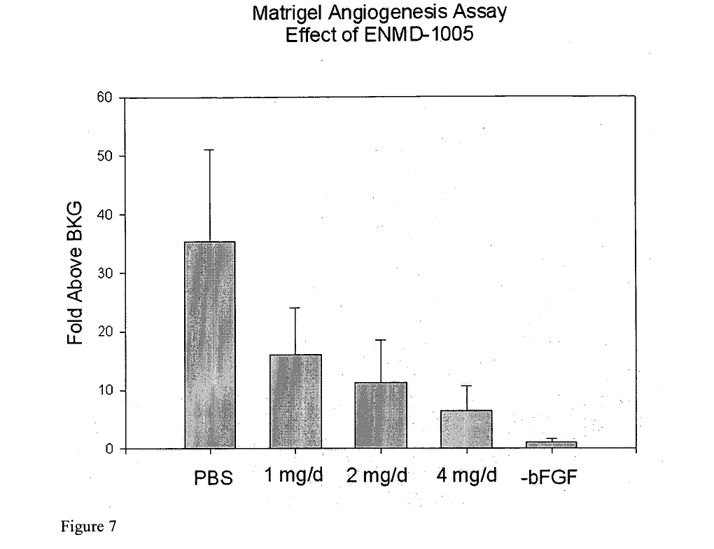

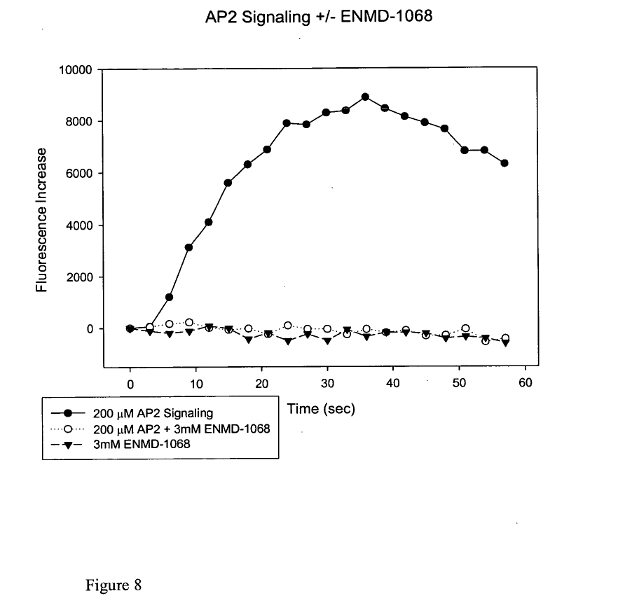

Matrigel plugs from animals treated with LIGK (ENMD-1005) (SEQ ID NO:1) demonstrated a dose dependent inhibition of angiogenesis, based upon hemoglobin content in the plug (FIG. 7). At the highest dose of LIGK (ENMD-1005) (SEQ ID NO:1), angiogenesis was inhibited by more than 80%. These data demonstrate that LIGK (ENMD-1005) (SEQ ID NO:1) has potent antiangiogenic activity, and further suggest a mechanism by which LIGK (ENMD-1005) (SEQ ID NO:1) could block tumor growth.

### Example 6

### Effect of LIGK (ENMD-1005) (SEQ ID NO:1) on Arthritis in Mice

On day 0, BALB/c mice were injected intravenously with the 1-2 mg 1B11 monoclonal anti-collagen II antibody. On day 1, animals were injected intraperitoneally with 20 μg LPS, and treatment with PAR-2 antagonists (200 mg/kg/day, intraperitoneally) for 7 days. After treatment was completed, disease is quantified by measuring the thickness (swelling) in both hind feet of the mouse. This was compared to untreated mice (p<0.05 vs. vehicle control).

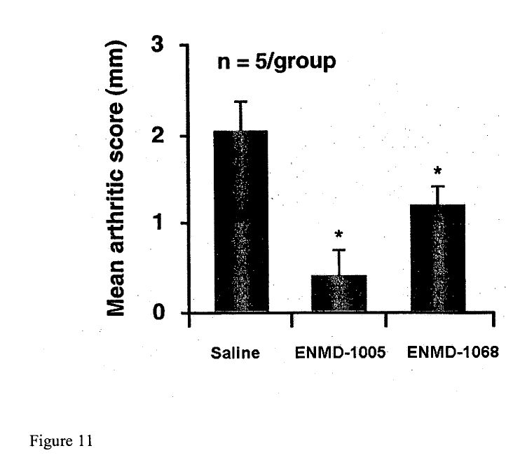

Both ENMD-1068 and LIGK (ENMD-1005) (SEQ ID NO:1) inhibited inflammation. FIG. 11 shows attenuation of arthritis in the presence of LIGK (ENMD-1005) (SEQ ID NO:1) and ENMD-1068.

### Example 7

### Prevention of Arthrogen-CIA Induced Body Weight Loss in Mice

On day 0, BALB/c mice were injected i.v. with the 1-2 mg 1B11 monoclonal anti-collagen II antibody. On day 1, animals were injected intraperitoneally with 20 μg LPS, and treatment with PAR-2 antagonists (200 mg/kg/day intraperitoneally) for 7 days. After treatment was completed, disease is quantified by measuring the thickness (swelling) in both hind feet of the mouse. This was compared to untreated mice. This model results in significant weight loss associated with the administration of LPS. Treatment of these mice with LIGK (ENMD-1005) (SEQ ID NO:1) abrogated this LPS induced weight loss.

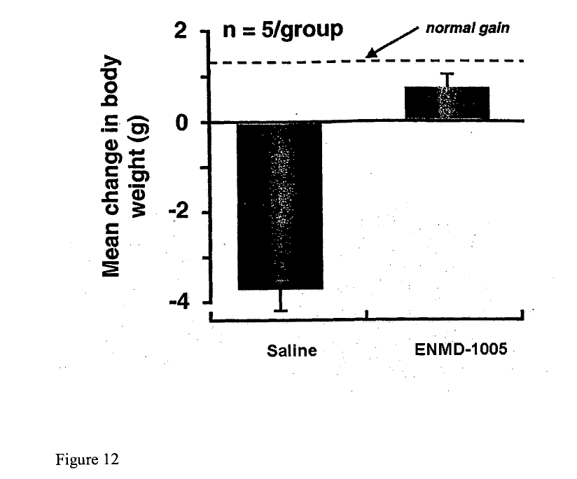

FIG. 12 shows prevention of weight loss in the presence of LIGK (ENMD-1005) (SEQ ID NO:1).

### Example 8

### In Vivo and in Vitro Activity of ENMD-1068

ENMD-1068 was discovered to be an inhibitor of PAR-2 signaling in vitro (FIG. 9). Like the LIGK (ENMD-1005) (SEQ ID NO:1) peptide, ENMD-1068 has no inhibitory effects on signaling by ATP (FIG. 9) or PAR-1 (not shown). Taken together, the identification of a second specific PAR-2 inhibitor, due to its enhanced activity, provides insight into the design and synthesis of other PAR-2 antagonist molecules.

### Example 9

### General Schemes for Synthesis of Piperazines

These products were obtained by coupling piperazine with the respective side chains using amide coupling reactions such as DCC/HOBT. The amine side chains were protected with either t-Boc or Cbz, and were removed after the coupling reactions using standard conditions.

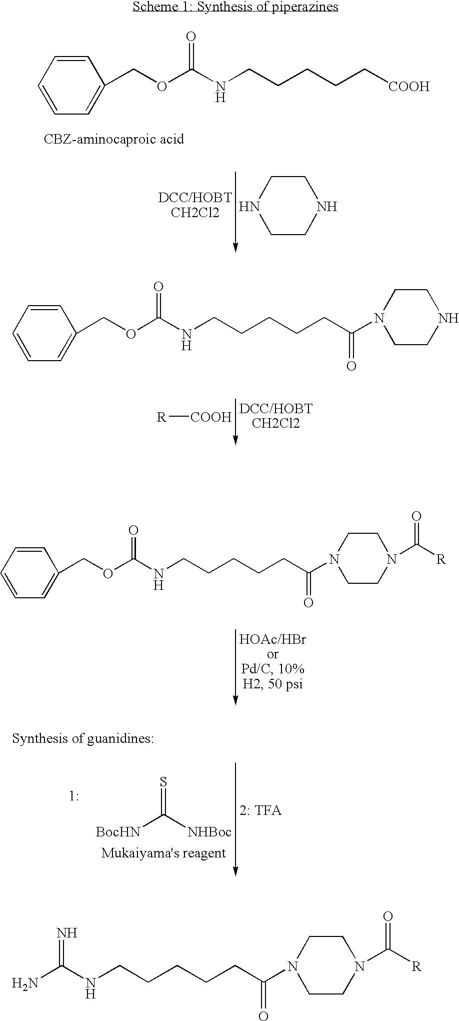

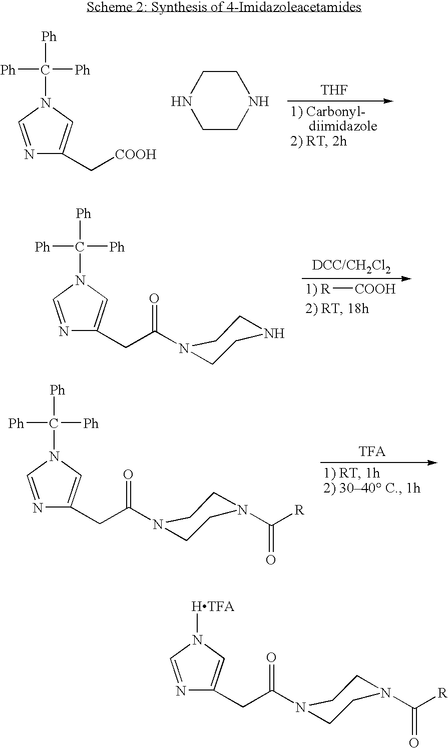

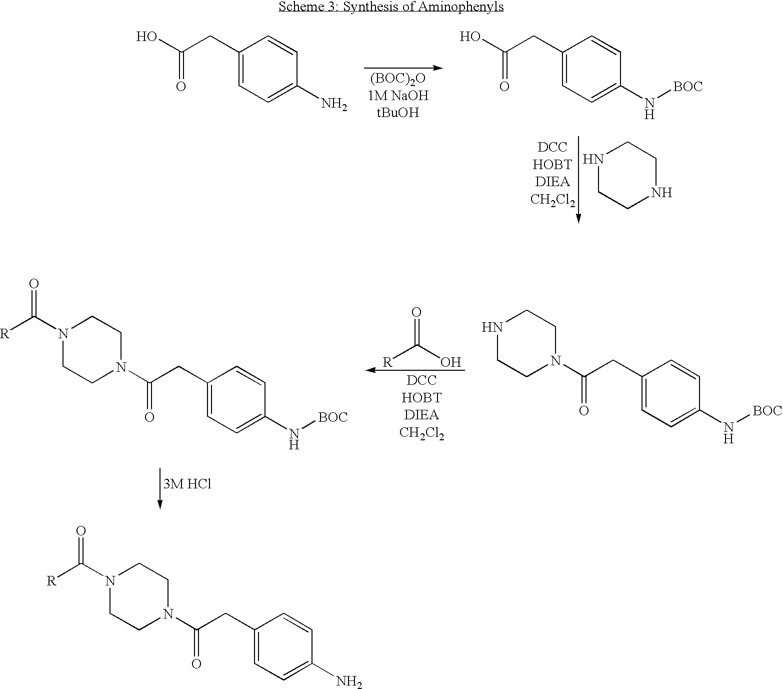

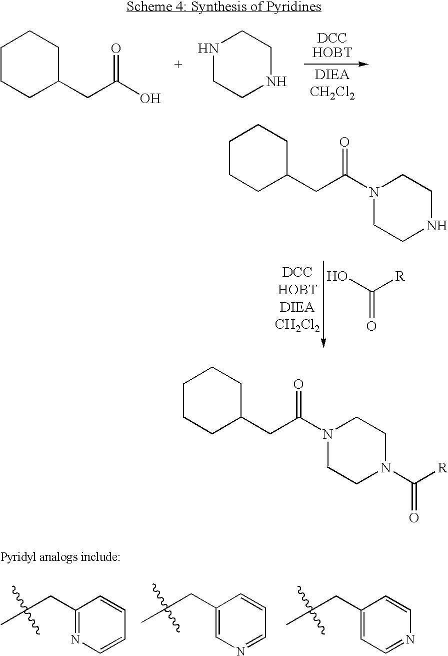

### Synthesis of ENMD-1033: N′-[S-2-methylbutanoyl]-N⁴-[6′-aminohexanoyl]-piperazine

Boc-6-aminohexanoyl-piperazine (obtained by reaction of piperazine with Boc-Aha using diisopropylcarbodiimide+HOBt) was reacted with S-2-methyl butanoic acid chloride. The Boc group was cleaved using TFA and product was converted into hydrochloride by treatment with HCl/THF and lyophilization of aqueous solution to yield the required compound.

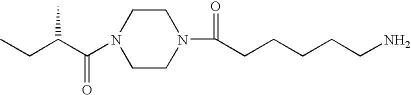

### Synthesis of ENMD-1034: N¹-[R-2-methylbutanoyl]-N⁴-[6′-aminohexanoyl]-piperazine

Boc-6-aminohexanoyl-piperazine was reacted with R-2-methyl butanoic acid chloride. The Boc group was cleaved using TFA and product was converted into hydrochloride by treatment with HCl/THF and lyophilization to yield the required compound.

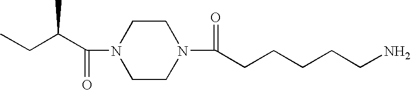

### Synthesis of ENMD-1035: N′-[2′-methylpropanoyl]-N⁴-[6′-aminohexanoyl]-piperazine

Boc-6-aminohexanoyl-piperazine was reacted with 2-methylpropanoic acid chloride. The Boc group was cleaved using TFA and product was converted into hydrochloride by treatment with HCl/THF and lyophilization to yield the required compound.

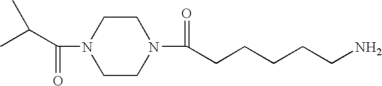

### Synthesis of ENMD-1036: N¹-butanoyl-N-4-[6′-aminohexanoyl]-piperazine

Boc-6-aminohexanoyl-piperazine was reacted with butanoic acid chloride. The Boc group was cleaved using TFA and product was converted into hydrochloride by treatment with HCl/THF and lyophilization to yield the required compound.

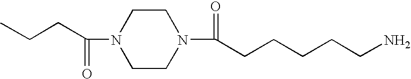

### Synthesis of ENMD-1037: N′-propanoyl-N-4-[6′-aminohexanoyl]-piperazine

Boc-6-aminohexanoyl-piperazine was reacted with propanoic acid chloride. The Boc group was cleaved using TFA and product was converted into hydrochloride by treatment with HCl/THF and lyophilization to yield the required compound.

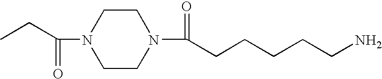

### Synthesis of ENMD-1038: N¹-[5′-methylhexanoyl]-N⁴-[6′-aminohexanoyl]-piperazine

Boc-6-aminohexanoyl-piperazine was reacted with 5-methylhexanoic acid chloride. The Boc group was cleaved using TFA and product was converted into hydrochloride by treatment with HCl/THF and lyophilization to yield the required compound.

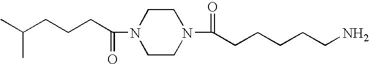

### Synthesis of ENMD-1039: N¹-hexanoyl-N⁴-[6-aminohexanoyl]-piperazine

Boc-6-aminohexanoyl-piperazine was reacted with hexanoic acid chloride. The Boc group was cleaved using TFA and product was converted into hydrochloride by treatment with HCl/THF and lyophilization to yield the required compound.

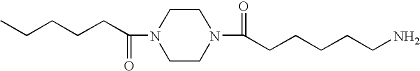

### Synthesis of ENMD-1040: N¹-pentanoyl-N-4-[6′-aminohexanoyl]-piperazine

Boc-6-aminohexanoyl-piperazine was reacted with pentanoic acid chloride. The Boc group was cleaved using TFA and product was converted into hydrochloride by treatment with HCl/THF and lyophilization to yield the required compound.

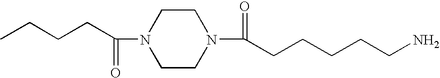

### Synthesis of ENMD-1041: N¹-[4′-methylpentanoyl]-N⁴-[6′-aminohexanoyl]-piperazine

Boc-6-aminohexanoyl-piperazine was reacted with 4-methyl pentanoic acid chloride. The Boc group was cleaved using TFA and product was converted into hydrochloride by treatment with HCl/THF and lyophilization to yield the required compound.

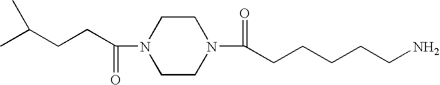

### Synthesis of ENMD-1065: N¹-3-methylbutyryl-N⁴-[2-(4-aminophenyl)-ethanoyl]-piperazine

Piperazine was coupled with t-Boc protected 4-aminophenylacetic acid using DCC/HOBT in CH₂Cl₂, and then coupled again with isovaleric acid with DCC/HOBT in CH₂Cl₂. Boc protection group was then removed using 3M HCl in EtOAc/MeOH to give product.

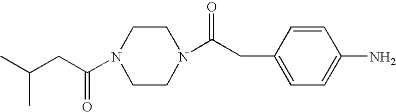

¹H NMR (300 MHz, CDCl₃) δ 7.04 (d, J=8.29 Hz, 2H), 6.66 (d, J=8.29 Hz, 2H), 3.69-3.53 (m, 6H), 3.52-3.48 (br s, 1H), 3.47-3.39 (m, 4H), 3.28-3.19 (br s, 1H), 2.26-2.03 (m, 3H), 0.97 (d, J=6.4 Hz, 6H).

### Synthesis of ENMD-1066: 1-(4-(2-(1H-imidazol-4-yl)acetyl)piperazin 1-yl)-3-methylbutan-1-amide hydrochloride

Synthesis according to the general Scheme 2 with 3-methylbutanoic acid to give 68% yield.

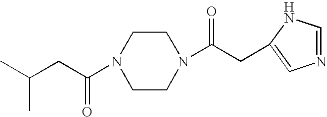

¹H NMR (300 MHz, DMSO-d₆) δ 14.05 (s, 2H, broad (amidazole-NH.HCl), 9.02 (s, 1H), 7.48 (s, 1H) 3.95 (s, 2H), 3.53 (m, 8H), 2.25 (m, 2H), 1.99 (m, 1H), 0.99-0.85 (d, 6H, J=6.6 Hz).

### Synthesis of ENMD-1067: 4-(3-methylbutanoyl)piperazin-1-(5′-carbamoyl-pentylguanidine)

Synthesis according to the general Scheme 1 with 3-methylbutyric acid, 6-cbz-amino-hexanoic acid, and boc-thiourea to give 65% yield.

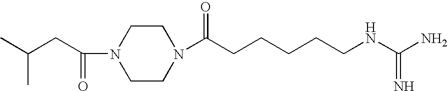

¹H NMR (300 MHz, methanol-d4) δ 3.70-3.53 (m, 8H), 3.18 (t, 2H, J=9 Hz), 2.49 (t, 2H, J=7.5 Hz), 2.35 (d, 2H, J=9 Hz), 2.15 (s, 4H), 2.05 (m, 1H), 1.70-1.55 (m, 4H), 1.50-1.34 (m, 2H), 1.00 (d, 6H, J=6.6 Hz).

### Synthesis of ENMD-1068: 1-(4-(3-methylbutanoyl)piperazin-1-yl)-(6′-aminohexan-1-amide) hydrobromide

Synthesis according to the general Scheme 1 with cbz-aminocaproic acid and 3-methylbutanoic acid to give 60% yield.

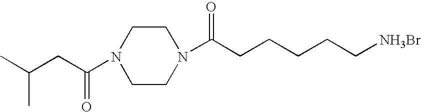

¹H NMR (300 MHz, DMSO-d₆) δ 7.63 (br s, 3H, —NH2.HBr), 3.52-3.36 (m, 8H), 2.85-2.69 (m, 2H), 2.32 (t, 2H, J=7.2 Hz), 2.21 (d, 2H, J=6.92 Hz), 1.98 (m, 1H), 1.160-1.44 (m, 4H), 1.38-1.25 (m, 2H), 0.90 (d, 6H, J=6.6 Hz).

### Synthesis of ENMD-1069: 1-(4-(2-cyclohexylacetyl)piperazin-1-yl)-2-(pyridin-2-yl)-acetaldehyde

Synthesis according to general Scheme 4 with pyrid-2-ylacetic acid.

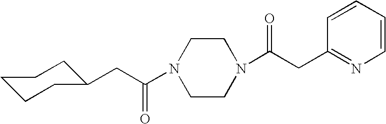

¹H NMR (300 MHz, CDCl₃) δ 8.44 (dq, J=5.1, 1.0 Hz, 1H), 7.58 (t, J=7 Hz, 1H), 7.32-7.22 (m, 1H), 7.11 (t, J=6 Hz, 1H), 3.87 (s, 2H), 3.63-3.25 (m, 8H), 2.18-2.06 (m, 2H), 1.81-1.48 (m, 5H), 1.30-0.73 (m, 6H).

### Synthesis of ENMD-1070: N¹-2-cyclohexylethanoyl-N⁴-[2-(4-aminophenyl)-ethanoyl]-piperazine

Piperazine was coupled with t-Boc protected 4-aminophenylacetic acid using DCC/HOBT in CH₂Cl₂, and then coupled again with 2-cyclohexylacetic acid with DCC/HOBT in CH₂Cl₂. Boc protection group was then removed using 3M HCl in EtOAc/MeOH to give product.

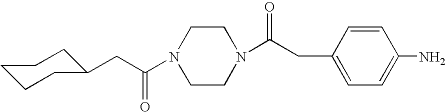

¹H NMR (300 MHz, CDCl₃) δ 7.03 (d, J=8.29 Hz, 2H), 6.65 (d, J=8.29 Hz, 2H), 3.69-3.54 (m, 7H), 3.47-3.38 (m, 4H), 3.28-3.19 (m, 1H), 2.25-2.12 (m, 2H), 1.85-1.59 (m, 6H), 1.37-0.83 (m, 5H).

### Synthesis of ENMD-1071: 1-(4-(2-(1H-imidazol-4-yl)acetyl)piperazin-1-yl)-1-cyclohexyl-acetamide hydrochloride

Synthesis according to the general Scheme 2 with cyclohexylacetic acid to give 68% yield.

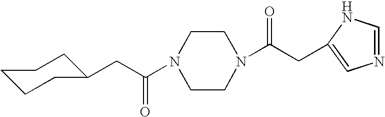

¹H NMR (300 MHz, DMSO-d₆) δ 14.06 (s, 2H, Broad—(imidazole-NH.HCl), 9.05 (s, 1H), 7.49 (s, 1H) 3.95 (d, 2H, J=3.2 Hz), 3.53 (m, 8H), 2.25 (m, 2H), 1.75-1.50 (m, 6H), 1.32-1.01 (m, 2H), 0.95-0.85 (t, 2H, J=6.2 Hz).

### Synthesis of ENMD-1072: 4-(1-cyclohexylacetyl)piperazin-1-(5′-carbamoyl-pentylguanidine)

Synthesis according to the general Scheme 1 with cyclohexylacetic acid, cbz-aminohexanoic acid, and boc-thiourea to give 55% yield.

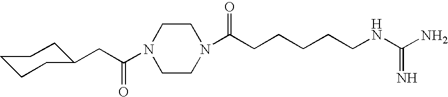

¹H NMR (300 MHz, methanol-d4) δ 3.68-3.53 (m, 8H), 3.36 (s, 4H), 3.19 (t, 2H, J=7 Hz), 2.47 (t, 2H, J=7 Hz), 2.32 (d, 2H, J=7 Hz), 1.82-1.56 (m, 6H), 1.50-0.94 (m, 5H).

### Synthesis of ENMD-1073: N′-(2-cyclohexylethanoyl)-N⁴-(6-aminohexanoyl)piperazine

Piperazine was coupled with cbz-6-aminocaproic acid using DCC/HOBT in CH₂Cl₂, and then coupled again with 2-cyclohexylacetic acid with DCC/HOBT in CH₂Cl₂. Cbz protection group was then removed with Pd—C (10%) in EtOAc at 50 psi of H₂ gas to give ENMD-1073 in 63% yield.

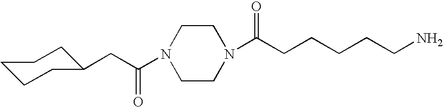

¹H NMR (300 MHz, CDCl₃) δ 3.71-3.59 (m, 4H), 3.55-3.42 (m, 4H), 2.74 (t, J=6.59 Hz, 2H), 2.37 (t, J=7.35 Hz, 2H), 2.25 (d, J=6.79 Hz, 2H), 1.88-0.88 (m, 17H).

### Synthesis of ENMD-1074: 1-(4-(2-cyclohexylacetyl)piperazin-1-yl)-2-(pyridin-3-yl)-acetaldehyde

Synthesis according to general Scheme 4 with pyrid-3-ylacetic acid.

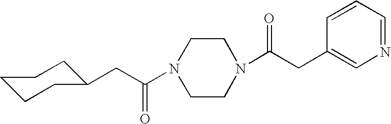

¹H NMR (300 MHz, CDCl₃) δ 8.58-8.49 (m, 2H), 7.69-7.60 (m, 1H), 7.35-7.24 (m, 1H), 3.76 (m, 2H), 3.72-3.35 (m, 8H), 2.29-2.16 (m, 2H), 1.90-1.59 (m, 5H), 1.40-0.86 (m, 6H).

### Synthesis of ENMD-1075: N¹-2-phenylethanoyl-N⁴-[2-(4-aminophenyl)-ethanoyl]-piperazine

Piperazine was coupled with t-Boc protected 4-aminophenylacetic acid using DCC/HOBT in CH₂Cl₂, and then coupled again with 2-phenylacetic acid with DCC/HOBT in CH₂Cl₂. Boc protection group was then removed using 3M HCl in EtOAc/MeOH to give product.

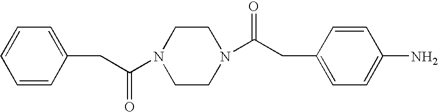

¹H NMR (300 MHz, CDCl₃) δ 7.38-7.19 (m, 5H), 7.02 (d, J=7.73 Hz, 2H), 6.65 (d, J=8.48 Hz, 2H), 3.78-3.69 (m, 2H), 3.65-3.57 (m, 2H), 3.49-3.36 (m, 6H), 3.24-3.15 (s, 2H), 1.69 (br s, 2H).

### Synthesis of ENMD-1076: 1-(4-(2-(1H-imidazol-4-yl)acetyl)piperazin-1-yl)-1-benzyl-amide

Synthesis according to the general Scheme 2 with phenylacetic acid to give 75% yield.

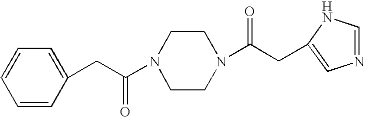

¹H NMR (300 MHz, CDCl₃) δ 7.065 (s, 1H), 7.35-7.15 (m, 5H), 6.95 (s, 1H), 3.82-3.68 (m, 4H), 3.58 (s, 4H), 3.35 (s, 4H).

### Synthesis of ENMD-1077: 4-phenylacetoyl-piperazin-1-(5′-carbamoyl-pentylguanidine)

Synthesis according to the general Scheme 1 with phenylacetic acid, cbz-aminocaproic acid, and boc-thiourea to give 68% yield.

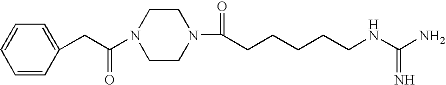

¹H NMR (300 MHz, methanol-d4) δ 7.38-7.23 (m, 5H), 3.83 (s, 2H), 3.70-3.50 (m, 5H), 3.50-3.38 (m, 2H), 3.36(s, 2H), 3.18 (t, 2H, J=7 Hz), 1.70-1.50 (m, 6H), 1.48-1.29 (m, 2H).

### Synthesis of ENMD-1078: N¹-(2-phenylethanoyl)-N⁴-(6-aminohexanoyl)piperazine

Piperazine was coupled with cbz-6-aminocaproic acid using DCC/HOBT in CH₂Cl₂, and then coupled again with 2-phenylacetic acid with DCC/HOBT in CH₂Cl₂. Cbz protection group was then removed with Pd—C (10%) in EtOAc at 50 psi of H₂ gas to give ENMD-1078 in 50% yield.

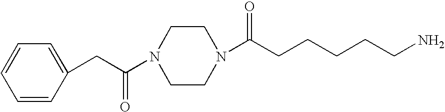

¹H NMR (300 MHz, CDCl₃) δ 7.40-7.22 (m, 5H), 3.78 (s, 2H), 3.72-3.54 (m, 4H), 3.48-3.38 (m, 5H), 3.28-3.17 (m, 1H), 2.74 (br s, 2H), 2.39-2.22 (m, 2H), 1.71-1.57 (m, 2H), 1.57-1.45 (m, 2H), 1.45-1.30 (m, 2H).

### Synthesis of ENMD-1079: 1-(4-(2-cyclohexylacetyl)piperazin-1-yl)-2-(pyridin-4-yl)-acetaldehyde

Synthesis according to general Scheme 4 with pyrid-4-ylacetic acid.

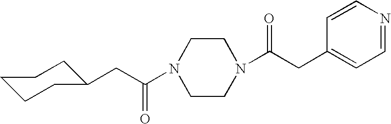

¹H NMR (300 MHz, CDCl₃) δ 8.59 (d, J=5.7 Hz, 2H), 7.21 (d, J=5.8 Hz, 2H), 3.76 (s, 2H), 3.72-3.33 (m, 8H), 2.28-2.14 (m, 2H), 1.88-1.58 (m, 5H), 1.38-0.86 (m, 6H).

### Synthesis of ENMD-1402: Methyl 6-(4-(3-methylbutanoyl)piperazin-1-yl)-6-oxohexanoate

5-(methoxycarbonyl)pentanoic acid was coupled to piperazine using DCC and HOBt. The resulting amide was coupled to isovaleric acid with DCC and HOBt.

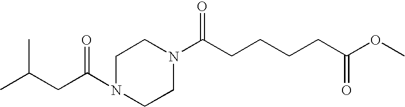

### Synthesis of ENMD 1403: 6-(4-(3-methylbutanoyl)piperazin-1-yl)-6-oxohexanoic acid

ENMD-1403 was prepared by hydrolysis of ENMD-1402 in methanolic KOH.

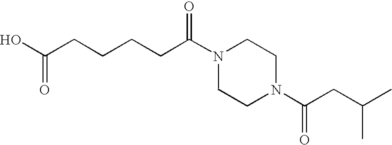

### Example 10

### General Schemes for Synthesis of Morpholines

General Procedure for EDC Coupling:

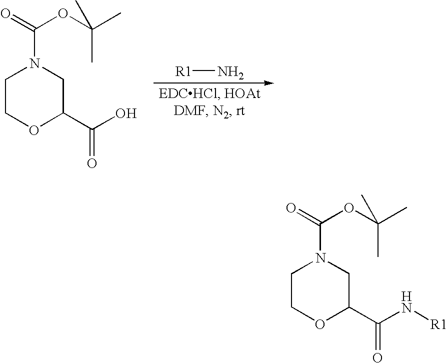

The amine (1 eq), the acid (1 eq), EDC.HCl (1.2 eq) and HOAt (1-hydroxy-7-azabenzotriazole, 1.2 eq) were dissolved in anhydrous DMF (20 vol) and stirred under N₂ at room temperature. The reaction was monitored both by TLC and LC-MS. Once the reaction was complete, water (25 vol) and ethyl acetate (15 vol) were added and both layers separated. The aqueous layer was extracted with ethyl acetate (3×15 vol) and the combined organic layers were washed with brine, dried over Na₂SO₄ and filtered. After solvent removal the crude was purified by column chromatography (mixtures ethyl acetate-heptane), affording the pure amides in yields ranging from 80% to quantitative.
General Procedure for t-Boc Deprotection:

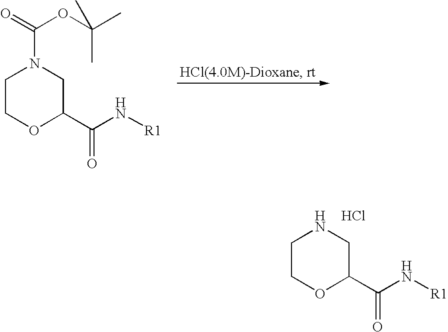

The N-Boc morpholine carboxamide was dissolved in 10 vol of anhydrous HCl in Dioxane (4.0M) and stirred at room temperature for a few hours. Once the reaction was complete, the solvents were removed under vacuo to afford the morpholine salt as a powdery solid in quantitative yields. The crude product was generally used without further purification for the next step.
General Procedure for Capping with Acid Chlorides:

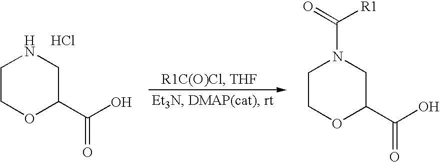

The acid chloride (1.1 eq) was added to a suspension of the starting material salt in THF-Et₃N (20 vol; 20:1), stirred at 0° C. under N₂. The ice bath was allowed to reach room temperature and the reaction monitored by TLC (mixtures ethyl acetate-Heptane) and/or LC-MS.

After completion the reaction mixture was poured into a saturated aqueous solution of NH₄Cl, both layers separated and the aqueous further extracted with DCM (3×). The combined organic layers were washed with brine solution (2×), dried over MgSO₄ and the solvent removed under vacuo. The crude was purified by column chromatography (mixtures ethyl acetate-Heptane and final flushing with MeOH-ethyl acetate) afforded the desired amide in yields typically ˜50%.
General Procedures for TBTU Coupling:

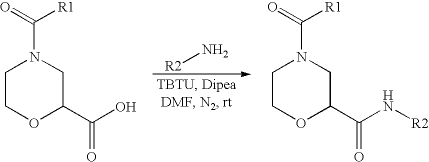

The starting material acid, TBTU (O-benzotriazole-1-yl-N,N,N′N′-tetramethyl uranium tetrafluoroborate, 1.0 eq), Dipea (1.0 eq) and the amine (1.0 eq) were dissolved in anhydrous DMF (20 vol) and stirred under N₂ at rt.

The reaction was monitored by LC-MS and once the reaction was complete, ethyl acetate-water was added (1:1, 30 vol) and both layers separated. The aqueous layer was further extracted with ethyl acetate (3×) and the combined organic layers were washed with brine (2×), dried over Na₂SO₄ and the solvent removed under vacuo. The crude was purified by column chromatography (mixtures ethyl acetate-heptane; final ethyl acetate-MeOH flush), afforded the amides in yields ranging from 80% to quantitative.

### Synthesis of ENMD-1521: 4-(2-Cyclohexyl-acetyl)-morpholine-2-carboxylic acid (6-amino-hexyl)-amide

Morpholine carboxylic acid amine salt was first coupled with 2-cyclohexylacetyl chloride. The acid was then coupled with mono-t-Boc-diaminohexane using TBTU and deprotected to yield ENMD-1521.

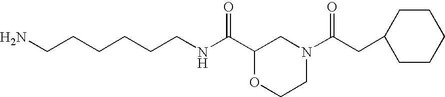

LCMS m/z 354(MH⁺). ¹H NMR (400 MHz, methanol-d4) δ ppm 4.68-4.76 (0.5H, m), 4.19-4.29 (0.5H, m), 3.95-4.17 (2H, m), 3.84-3.94 (1H, m), 3.54-3.66 (1H, m), 3.19-3.36 (3H, m), 2.90-3.04 (2.5H, m), 2.63-2.74 (0.5H, m), 2.27-2.42 (2H, m), 1.14-1.86 (17H, m), 0.95-1.12 (2H, m).

### Synthesis of ENMD-1522: 4-(3-Methyl-butyryl)-morpholine-2-carboxylic acid (6-amino-hexyl)-amide

Morpholine carboxylic acid amine salt was first coupled with isobutyl acid chloride. The acid was then coupled with mono-t-Boc-diaminohexane using TBTU and deprotected to yield ENMD-1522.

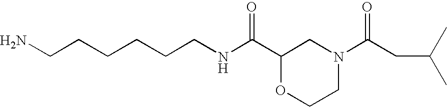

LCMS m/z 314(MH⁺). ¹H NMR (400 MHz, methanol-d4) δ ppm 4.69-4.77 (½H, m), 4.20-4.29 (½H, m), 3.95-4.16 (2H, m), 3.84-3.94 (1H, m), 3.54-3.67 (1H, m), 3.19-3.31 (4H, m), 2.89-3.03 (2H, m), 2.27-2.42 (2H, m), 2.03-2.16 (1H, m), 1.62-1.73 (2H, m), 1.52-1.61 (2H, m), 1.32-1.49 (4H,m), 0.94-1.05 (6H, m).

### Synthesis of ENMD-1523: 4-(6-Amino-hexanoyl)-morpholine-2-carboxylic acid cyclohexylmethyl-amide

Using the general schemes described, N-boc-morpholine carboxylic acid was coupled with 2-cyclohexyl aminoethane (cyclohexylmethylamine), deprotected, then coupled with N-boc-aminohexanoic acid. Final deprotection yielded ENMD-1523.

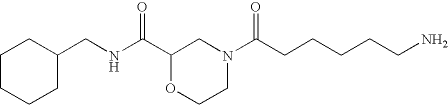

LCMS m/z 340(MH⁺). ¹H NMR (400 MHz, methanol-d4) δ ppm 4.66-4.76 (½H, m), 4.17-4.26 (½H, m), 3.97-4.12 (2H, m), 3.80-3.94 (1H, m), 3.55-3.69 (1H, m), 3.21-3.36 (2H, m), 2.92-3.16 (4H, m), 2.40-2.58 (2H, m), 1.61-1.81 (9H, m), 1.40-1.59 3H, m), 1.16-1.36 (3H, m), 0.88-1.03 (2H,m).

### Synthesis of ENMD-1524: 4-(6-Amino-hexanoyl)-morpholine-2-carboxylic acid isobutyl-amide

Using the general schemes described, N-boc-morpholine carboxylic acid was coupled with 2-methyl aminopropane (isobutylamine), deprotected, then coupled with N-boc-aminohexanoic acid. Final deprotection yielded ENMD-1524.

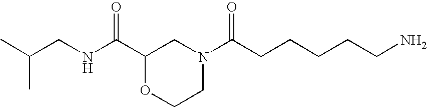

LCMS m/z 300(MH⁺). ¹H NMR (400 MHz, methanol-d4) δ ppm 4.51-4.59 (½H, m), 4.01-4.08 (½H, m), 3.82-3.97 (2H, m), 3.64-3.78 (1H, m), 3.39-3.53 (1H, m), 3.05-3.23 (2H, m), 2.75-2.94 (4H, m), 2.49-2.56 (1H, m), 2.24-2.41 (2H, m), 1.45-1.78 (5H, m), 1.24-1.36 (2H, m), 0.72-0.79 (6H, m).

### Example 11

### General Schemes for Synthesis of Benzimidazoles

General Procedure for Capping with Thioisocyanates:

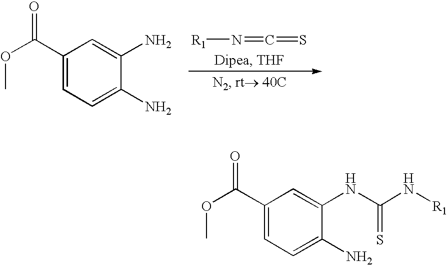

The thioisocyanate (1.1 eq) was added to a solution of the starting material diaminobenzene and Dipea (diisopropylethylamine, 1.5 eq) in dry THF (10 vol), stirred at 40° C. under N₂. The reaction was monitored by LC-MS. Once the reaction was complete, the mixture was cooled to rt and the excess of thioisocyanate scavenged with PAM-resin. Filtration and removal of the solvent under vacuo afforded the crude thioureas in quantitative yields.
General Method for Cyclization:

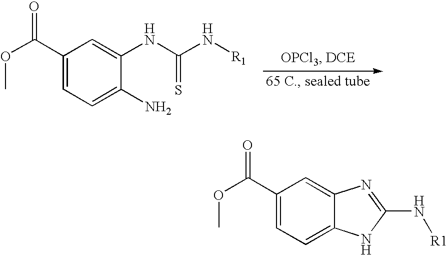

The thiourea and POCl₃ (3.0 eq) were dissolved in anhydrous dichloroethane (DCE, 20 vol) and the mixture stirred at rt for 5 min in a sealed tube. Then the reaction was heated up to 65° C. and its progress monitored by LC-MS. Once the reaction was complete, the mixture was poured into ice-water (7:3) and stirred vigorously. The acidic aqueous layer (pH˜3) was extracted with dichloromethane (DCM, 3×). Organic layers were dried over MgSO₄, filtered and solvent removed in vacuum. The crude was purified by column chromatography [ethyl acetate-Heptane, gradients from 3:7 to neat ethyl acetate; ethyl acetate-iPrOH and a final neat iPrOH flush] to afford the desired product in typical yields around 50%.
General Method for Saponification:

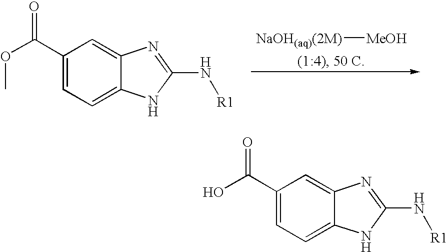

The methyl ester was dissolved in MeOH (4 vol), 1 vol of an aqueous solution of NaOH (2.0M) was then added and the mixture heated to 50° C. Once the hydrolysis was completed, the reaction mixture was cooled to rt, the pH adjusted to 6-7 with HCl (0.5N) and the MeOH removed in vacuum. The aqueous layer was extracted with DCM (3×), the combined organic layers were dried over MgSO₄ and filtered. After solvent removal the crude acids were obtained in moderate to good yields and in an average purity of 95% by UV.

TBTU Coupling:

See General Procedure from the Morpholine Scheme.

Boc Deprotection:

See General Procedure from the Morpholine Scheme.

### Synthesis of ENMD-1525: 2-Isobutylamino-3H-benzoimidazole-5-carboxylic acid (6-amino-hexyl)-amide

Using the general schemes provided, 1,2-diaminobenzene-4-carboxylic acid methyl ester was coupled with 2-methylpropane thioisocyanate, cyclized with POCl₃ and saponified. This intermediate was then coupled with mono-N-Boc-diaminohexane and deprotected to yield ENMD-1525.

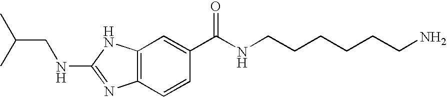

LCMS m/z 332 (MH⁺). ¹H NMR (400 MHz, methanol-d4) δ ppm 7.56 (1H, s), 7.39 (1H, d, J=8.3 Hz), 7.09 (1H, d, J=8.3 Hz), 3.44-3.69 (3H, m), 3.23-3.31 (3H, m), 3.09 (2H, d, J=7.0 Hz), 2.60 (1H, t, J=7.3 Hz), 1.74-1.94 (1H, m), 1.15-1.63 (9H, m), 0.90 (6H, d, J=6.7 Hz).

### Synthesis of ENMD-1526: 2-(Cyclohexylmethyl-amino)-3H-benzoimidazole-5-carboxylic acid (6-amino-hexyl)-amide

Using the general schemes provided, 1,2-diaminobenzene-4-carboxylic acid methyl ester was coupled with cyclohexylmethyl thioisocyanate, cyclized with POCl₃ and saponified. This intermediate was then coupled with mono-N-Boc-diaminohexane and deprotected to yield ENMD-1526.

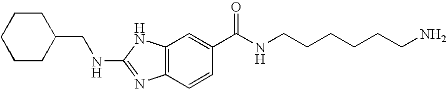

LCMS m/z 372 (MH⁺). ¹H NMR (400 MHz, methanol-d4) δ ppm 7.56 (1H, d, J=1.6 Hz), 7.39 (1H, dd, J=8.3, 1.6 Hz), 7.09 (1H, d, J=8.3 Hz), 3.41-3.70 (4H, m), 3.23-3.34 (4H, m), 3.11 (2H, d, J=7.0 Hz), 2.51-2.65 (1H, m), 1.75 (2H, d, J=13.0 Hz), 1.63-1.71 (2H, m), 1.48-1.63 (4H, m), 1.04-1.48 (9H, m), 0.77-0.98 (2H, m).

### Synthesis of ENMD-1553: 2-(6-Amino-hexylamino)-3H-benzoimidazole-5-carboxylic acid cyclohexylmethyl-amide

Using the general schemes provided, 1,2-diaminobenzene-4-carboxylic acid methyl ester was coupled with Boc-N-aminohexane thioisocyanate, cyclized with POCl₃ and saponified. This intermediate was then coupled with cyclohexylmethylamine and deprotected to yield ENMD-1553.

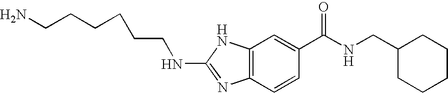

LCMS m/z 372 (MH⁺). ¹H NMR (400 MHz, methanol-d4) 8 ppm 6.63-8.12 (3H, m), 3.39 (2H, t, J=7.09 Hz), 3.21 (2H, d, J=7.34 Hz), 2.78-2.96 (2H, m), 1.55-1.89 (8H, m), 1.19-1.53 (9H, m), 0.76-1.12 (3H, m).

### Example 12

### General Schemes for Synthesis of Biaryls

Synthesis of Biphenyl Alkylchlorides:

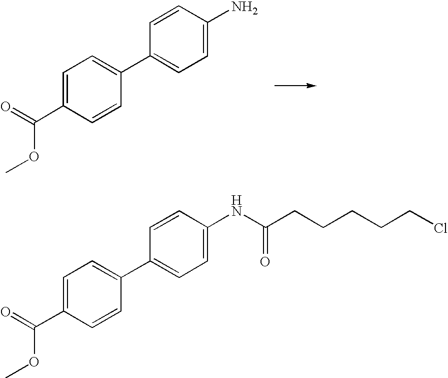

To a solution of 4′-amino-biphenyl-4-carboxylic acid methyl ester (200 mg, 0.88 mmol) and DIPEA (0.30 ml, 1.76 mmol, 2 eq.) in DCM (3 ml) was added dropwise a solution of chloro-acid chloride (156 mg, 0.92 mmol, 1.05 eq.) in DCM (1 ml). The reaction was left under stirring for 6 hours. LCMS analysis showed complete consumption of starting material and product observed as main peak. Water added, solution acidified with 1N HCl, DCM extraction (2×). Combined organic layers were washed with water, brine, dried over Na₂SO₄, filtered and concentrated. The residue was purified by column chromatography over silica eluted with DCM then from 1 to 2% MeOH/7N NH₃ in DCM to yield product as a beige solid (312 mg, 98% yield). LCMS m/z 360 (MH⁺).
General Procedure for Biaryl Capping Using HOAt and EDC:

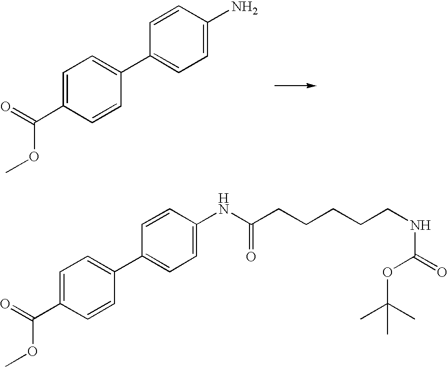

To solution of N-Boc amino hexanoic acid (370 mg, 1.58 mmol, 1.2 eq.), HOAt (220 mg, 1.58 mmol, 1.2 eq.), EDC.HCl (300 mg, 1.58 mmol, 1.2 eq.) in DMF (3 ml) was added a solution of 4′-Amino-biphenyl-4-carboxylic acid methyl ester (300 mg, 1.32 mmol) in DMF (2 ml). The reaction was left at room temperature overnight. Water was added and EtOAc extraction (2×). The combined organic layers were washed with water (2×), brine, dried over Na₂SO₄, filtered and concentrated. Purification over silica eluted with a gradient of DCM-MeOH/7N NH₃. From 1 to 3% of MeOH/7N NH₃ in DCM to yield product as a beige solid (437 mg, 75% yield).

LCMS m/z 341 (MH⁺-Boc group). ¹H NMR (400 MHz, CDCl₃) δ ppm 8.02 (2H, d, J=8.6 Hz), 7.50-7.60 (6H, m), 7.30-7.40 (1H, br), 4.45-4.60 (1H, br), 3.87 (3H, s, CH₃), 3.07 (2H, q, J=6.5 Hz), 2.32 (2H, t, J=7.6 Hz), 1.71 (2H, pent, J=7.5 Hz), 1.47 (2H, pent, J=7.3 Hz), 1.30-1.42 (11H, m).
General Procedure for the Hydrolysis of Methyl Esters with LiOH.H₂O:

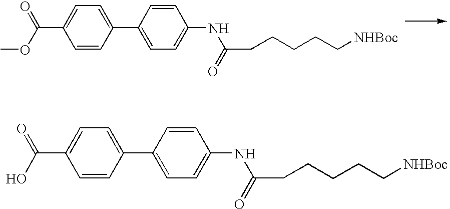

To a solution of 4′-(6-tert-Butoxycarbonylamino-hexanoylamino)-biphenyl-4-carboxylic acid methyl ester (400 mg, 0.91 mmol) in THF (20 ml) was added a solution of LiOH.H₂O (230 mg, 5.45 mmol, 6 eq) in water (20 ml). Reaction left for 4-6 hours. LCMS shows complete hydrolysis (MH⁺=427 at 1.39 min). The reaction mixture was slightly acidified to acid pH with 1N HCl. TBME extraction (tert-butyl methyl ether, 2×). Combined organic layers were washed with water, brine, dried over Na₂SO₄, filtered and concentrated to yield product in good purity as a yellowish solid (355 mg, 92% yield).

LCMS m/z 427 (MH⁺). ¹H NMR (400 MHz, methanol-d4) δ ppm 7.97 (2H, d, J=8.3 Hz), 7.62 (2H, d, J=8.6 Hz), 7.59 (2H, J=9.0 Hz), 7.55 (2H, d, J=9.0 Hz), 2.95 (2H, t, J=6.8 Hz), 2.30 (2H, t, J=7.4 Hz), 1.63 (2H, pent, J=7.5 Hz), 1.42 (2H, pent, J=7.3 Hz), 1.27-1.37 (11H, m).
General Procedure for Conversion of Alkylchloride to Amine:

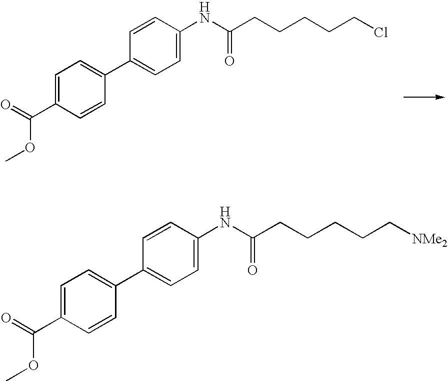

A solution 4′-(6-chloro-hexanoylamino)-biphenyl-4-carboxylic acid methyl ester (199 mg, 0.55 mmol), KI (370 mg, 2.21 mmol, 4 eq.), K₂CO₃ (306 mg, 2.21 mmol, 4 eq.) in dimethyl amine in THF (2.0 M, 10 ml) was sealed and heated to 90° C. overnight. Solution removed and residue taken in TBME and water. TBME extraction (2×). Combined organic layers were washed with water, brine, dried over Na₂SO₄, filtered and concentrated to yield product as a white solid in good purity without purification (196 mg, 96% yield).

LCMS m/z 369 (MH⁺). ¹H NMR (400 MHz, methanol-d4) 8 ppm 7.97 (2H, d, J=8.6 Hz), 7.64 (2H, d, J=8.6 Hz), 7.60 (2H, d, J=9.0 Hz), 7.56 (2H, d, J=9.0 Hz), 3.82 (3H, s, CH₃), 2.31 (2H, t, J=7.5 Hz), 2.25 (2H, t, J=7.8 Hz), 2.16 (6H, s, NMe₂), 1.65 (2H, pent, J=7.5 Hz), 1.47 (2H, pent, J=7.8 Hz), 1.27-1.37 (2H, m).
General Procedure for Conversion of Arylcarboxylic Acid to Amide:

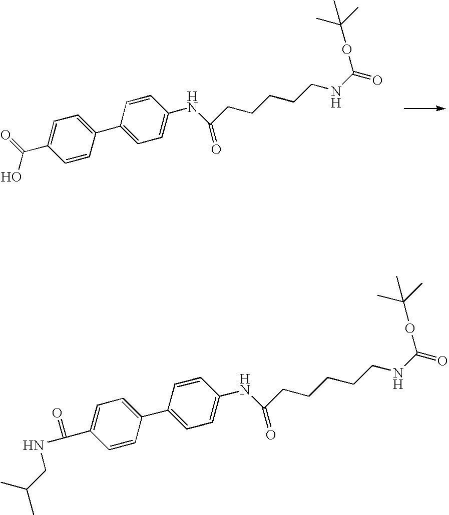

See general procedure for capping using HOAt and EDC. Materials used: 4′-(6-tert-butoxycarbonylamino-hexanoylamino)-biphenyl-4-carboxylic acid (150 mg, 0.35 mmol), HOAt (57.5 mg, 0.42 mmol, 1.2 eq.), EDC.HCl (81 mg, 0.42 mmol, 1.2 eq.), isobutylamine (35 μl, 0.35 mmol, 1.0 eq.) in DMF (3 ml). 159 mg (94%) of product isolated after column chromatography over silica eluted with DCM then from 4 to 15% MeOH in DCM. LCMS m/z 482 (MH⁺).

General Scheme for Boc Deprotection:

To a suspension of [5-(4′-isobutyl-carbamoyl-biphenyl-4-ylcarbamoyl)-pentyl]-carbamic acid tert-butyl ester (159 mg, 0.33 mmol) in dioxane (5.0 ml) was added hydrogen chloride in dioxane (4N, 5.0 ml). The reaction was left under stirring for 3-4 h. Solvent removed and residue taken in methanol and carbonate resin (˜10 fold) was added and mixture stirred for 3-4 hours. Filtration and concentration afforded 69.1 mg (55%) of product as a solid.

### Synthesis of ENMD-1527: 4′-(6-amino-hexanoylamino)-biphenyl-4-carboxylic acid isobutyl-amide

4′-Amino-biphenyl-4-carboxylic acid methyl ester was coupled to N-Boc-aminohexanoic acid. The ester was saponified and the resultant acid was coupled to isobutylamine using EDC. Deprotection of the Boc yielded ENMD-1527.

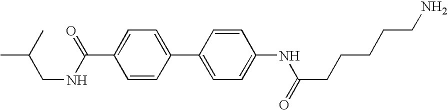

LCMS m/z 382 (MH⁺). ¹H NMR (400 MHz, methanol-d4) δ ppm 7.87 (2H, d, J=8.8 Hz), 7.58-7.76 (6H, m), 3.21 (2H, d, J=7.3 Hz), 2.64 (2H, t, J=7.1 Hz), 2.40 (2H, t, J=7.3 Hz), 1.85-2.04 (1H, m), 1.63-1.80 (2H, m), 1.35-1.61 (4H, m), 0.97 (6H, d, J=6.8 Hz).

### Synthesis of ENMD-1528: 4′-(6-amino-hexanoylamino)-biphenyl-4-carboxylic acid cyclohexylmethyl-amide

4′-Amino-biphenyl-4-carboxylic acid methyl ester was coupled to N-Boc-aminohexanoic acid. The ester was saponified and the resultant acid was coupled to cyclohexylmethylamine using EDC. Deprotection yielded ENMD-1528.

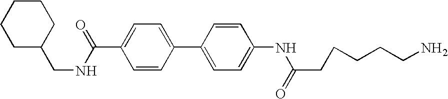

LCMS m/z 422 (MH⁺). ¹H NMR (400 MHz, MeOD) δ ppm 7.87 (2H, d, J=8.5 Hz), 7.58-7.74 (6H, m), 3.22 (2H, d, J=7.3 Hz), 2.59-2.72 (2H, t, J=7.1 Hz), 2.41 (2H, t,J=7.3 Hz), 1.60-1.87 (7H, m), 1.48-1.58 (2H, m), 1.37-1.47 (2H, m), 1.17-1.36 (4H, m), 1.02 (2H, m).

### Synthesis of ENMD-1529: 4′-(6-Dimethylamino-hexanoylamino)-biphenyl-4-carboxylic acid isobutyl-amide

4′-Amino-biphenyl-4-carboxylic acid methyl ester was coupled to 6-chloro-hexanoic acid chloride. The alkyl chloride was converted to the tertiary amine and the ester was saponified. The resultant acid was coupled to isobutylamine, and deprotection yielded ENMD-1529.

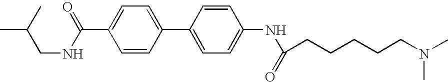

LCMS m/z 410 (MH⁺). ¹H NMR (400 MHz, MeOD) δ ppm 7.87 (2H, d, J=8.5 Hz), 7.54-7.75 (6H, m), 3.21 (2H, d, J=6.8 Hz), 2.30-2.44 (4H, m), 2.25 (6H, s), 1.92 (1H, hept, J=6.8 Hz), 1.75 (2H, quintet, J=7.5 Hz), 1.50-1.62 (2H, m), 1.32-1.46 (2H, m), 0.98 (6H, d, J=6.8 Hz).

### Synthesis of ENMD-1530: 4′-(6-Dimethylamino-hexanoylamino)-biphenyl-4-carboxylic acid cyclohexylmethyl-amide

4′-Amino-biphenyl-4-carboxylic acid methyl ester was coupled to 6-chloro-hexanoic acid chloride. The alkyl chloride was converted to the tertiary amine and the ester was saponified. The resultant acid was coupled to cyclohexylmethylamine and deprotected.

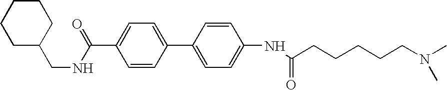

LCMS m/z 450 (MH⁺). ¹H NMR (400 MHz, MeOD) δ ppm 7.87 (2H, d, J=8.8 Hz), 7.56-7.77 (6H, m), 3.22 (2H, d, J=7.3 Hz), 2.28-2.47 (4H, m), 2.24 (6H, s), 1.60-1.87(8H, m), 1.50-1.61 (2H, m), 1.12-1.48 (5H, m), 0.90-1.11 (2H, m).

### Example 13

### General Schemes for Synthesis of Pyrazoles

General Amide Formation Via Acid Chloride:

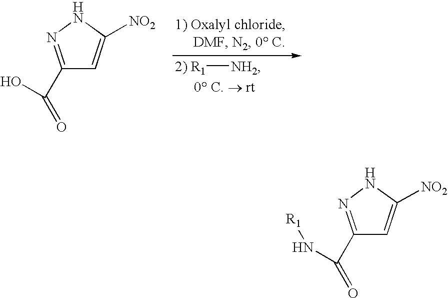

The starting material nitropyrazine carboxylic acid was dissolved in DMF and kept under N₂ at 0° C. Oxalyl chloride ((COCl)₂, 1.05 eq) was added and then, when the gas evolution ceased and the reaction mixture cleared, the amine (1.5 eq) was finally added. Once the reaction was complete, the mixture was poured onto aqueous saturated NH₄Cl solution and extracted with DCM (3×). The combined organic layers were washed with brine solution (2×), dried over MgSO₄ and the solvent removed in vacuum. The crude amides (typical yields above 70%) were used without further purification.

General Procedure for TBTU Coupling:

See Experimental Procedure for the Morpholines.
General Procedure for Nitro Reduction:

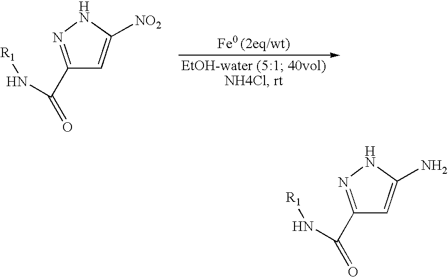

The nitropyrazole was dissolved in EtOH-water (5:1, 40 vol), Fe⁰ (2.0 eq) and ammonium chloride (1.0 eq) were then added. The resulting suspension was heated to 40° C. under N₂ and the reaction progress monitored by LC-MS. Once the reaction was complete (typically in a couple of hours), the mixture was filtered through celite while still warm and the cake washed thoroughly with EtOH (30 vol). The solvent was removed under vacuo and the crude residue was dissolved in ethyl acetate (30-50 vol), washed with water (3×15 vol) and brine (2×) and dried over Na₂SO₄. Removal of the solvent afforded the amino pyrazole in 50 to 94% yields, and were used without further purification.

General Procedure for Deprotection (i.e., Boc-Group Cleavage):

See General Procedure for the Morpholines.

### Synthesis of 1533: 5-(6-Amino-hexanoylamino)-1H-pyrazole-3-carboxylic acid isobutyl-amide

Nitropyrazine carboxylic acid was converted to an amide with oxalyl chloride and isobutylamine. The nitro was reduced and capped with N-Boc-aminohexanoic acid (N-t-Boc-6aminocaproic acid), and deprotection yielded ENMD-1533.

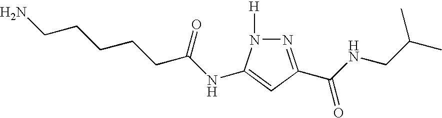

LCMS m/z 296 (MH⁺). ¹H NMR (400 MHz, DMSO d6) δ ppm 8.36-8.45 (1H, m), 7.04 (1H, s), 2.97-3.08 (2H, m), 2.47-2.56 (2H, m), 2.24-2.34 (2H, m), 1.74-1.87 (1H, m), 1.50-1.63 (2H, m), 1.18-1.42 (4H, m), 0.87 (6H, d, J=6.7 Hz).

### Synthesis of ENMD-1534: 5-(6-amino-hexanoylamino)-1H-pyrazole-3-carboxylic acid cyclohexylmethyl-amide

Nitropyrazine carboxylic acid was converted to an amide with oxalyl chloride and cyclohexylmethylamine. The nitro was reduced and capped with N-Boc-aminohexanoic acid, and deprotection yielded ENMD-1534.

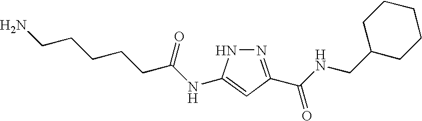

LCMS m/z 336 (MH⁺). ¹H NMR (400 MHz, DMSO d6) δ ppm 8.33-8.43 (1H, m), 7.04 (1H, s), 3.01-3.09 (2H, m), 2.46-2.57 (2H, m), 2.23-2.32 (2H, m), 1.43-1.73 (8H, m), 1.09-1.1.42 (7H, m), 0.82-0.97 (2H, m).

### Synthesis of ENMD-1550: 5-(3-methyl-butyrylamino)-1H-pyrazole-3-carboxylic acid (6-amino-hexyl)-amide

Nitropyrazine carboxylic acid was converted to an amide with oxalyl chloride and N-Boc-diaminohexane. The nitro was reduced and capped with isobutyric acid via TBTU, and deprotection yielded ENMD-1550.

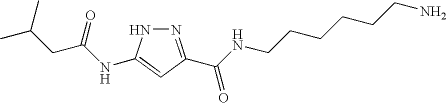

LCMS m/z 310 (MH⁺). ¹H NMR (400 MHz, MeOD) δ ppm 3.32-3.40 (4H, m), 2.86-2.95 (2H, m), 2.23-2.28 (2H, m), 2.07-2.22 (1H, m), 1.56-1.73 (4H, m), 1.36-1.50 (4H, m), 1.00 (6H, d, J=6.6 Hz).

### Synthesis of ENMD-1551: 5-(2-cyclohexyl-acetylamino)-1H-pyrazole-3-carboxylic acid (6-amino-hexyl)-amide

Nitropyrazine carboxylic acid was converted to an amide with oxalyl chloride and N-Boc-diaminohexane. The nitro was reduced and capped with cyclohexylacetic acid via TBTU, and deprotection yielded ENMD-1551.

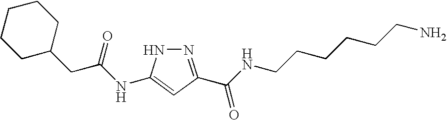

LCMS m/z 350 (MH⁺). ¹H NMR (400 MHz, MeOD) δ ppm 3.32-3.39 (3H, m), 2.88-2.94 (2H, m), 2.25 (2H, d, J=7.3 Hz), 1.79-1.90 (1H, m), 1.56-1.79 (9H, m), 1.39-1.49 (4H, m), 1.13-1.37 (3H, m), 0.93-1.12(2H, m).

### Example 14

### General Schemes for Synthesis of Isoxazoles

General Procedure for Synthesis of Azides:

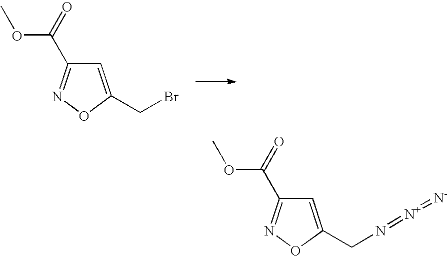

To a solution of 5-bromomethyl-isoxazole-3-carboxylic acid methyl ester (10.10 g, 5.0 mmol) in toluene (20 ml) was added TBAF (tetrabutylaminofluoride, 1.0 M, 0.5 ml, 10 mol %) and sodium azide (0.65 g, 10.0 mmol, 2 eq.). The reaction mixture was sealed and heated to 70° C. for 6 hours. TBME and water added to the cooled reaction mixture. TBME extraction (2×). Combined organic layers were washed with water (3×), brine, dried over Na₂SO₄, filtered and concentrated to yield 0.840 g (92%) of clean product. LCMS m/z 183 (MH⁺).
General Procedure for Saponification:

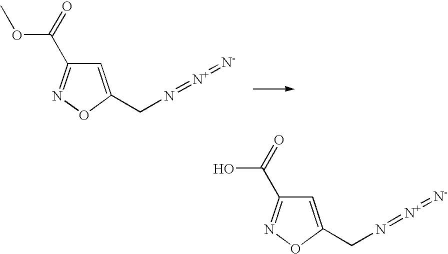

See general procedure for hydrolysis of methyl ester with LiOH.H₂O described for morpholines. Material used: 5-Azidomethyl-isoxazole-3-carboxylic acid methyl ester (0.84 g, 4.62 mmol), LiOH.H₂O (1.16 g, 27.7 mmol, 6 eq.), THF (10 ml), water (10 ml) to yield 0.775 g (100%) of product. LCMS m/z 214 (M+2Na⁺).
General Scheme for Amide Coupling:

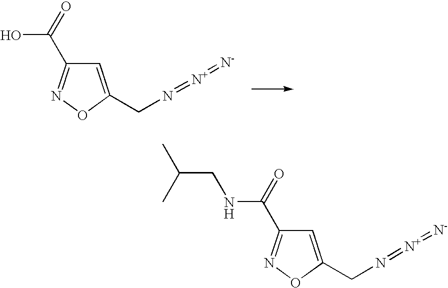

See general procedure for capping conditions using HOAt and EDC described for morpholines. Material used: 5-azidomethyl-isoxazole-3-carboxylic acid (150 mg, 0.89 mmol), HOAt (146 mg, 1.07 mmol, 1.2 eq.), EDC.HCl (205 mg, 1.07 mmol, 1.2 eq.), isobutyl amine (98 μl, 0.98 mmol, 1.1 eq.) in DMF (5 ml). 199 mg (100%) of product isolated after column chromatography over silica eluted with DCM then from 1:50 to 5:45 MeOH: DCM. LCMS m/z 224 (MH⁺).
General Procedure for the Catalytic Hydrogenation of the Azide Group:

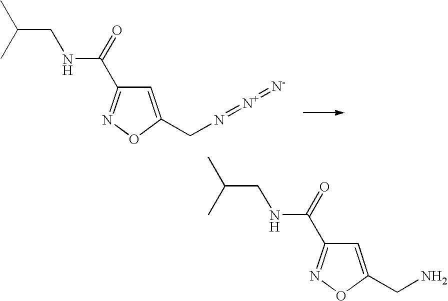

A solution of 5-Azidomethyl-isoxazole-3-carboxylic acid isobutyl-amide and Pd/C (10% w/wt, equal quantity of azide) in EtOH was hydrogenated at room temperature for 6 hours. Pd/C was filtered and washed with EtOH. The solution was concentrated to yield product as a solid. LCMS m/z 239 (MH+MeCN⁺).

### Synthesis of ENMD-1555: 5-[(5-amino-pentanoylamino)-methyl]-isoxazole-3-carboxylic acid isobutyl-amide

Following the general schemes provided, 5-bromomethyl-isoxazole-3-carboxylic acid was converted to an azide, saponified, coupled to isobutylamine, reduced to the amine, coupled to N-Boc-aminopentanoic acid, and deprotected.

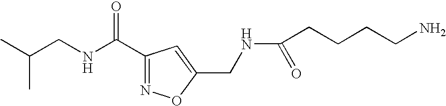

LCMS m/z 297(MH⁺). ¹H NMR (400 MHz, methanol-d4) δ ppm 6.57 (1H, s), 4.53 (2H, s), 3.17 (2H, d, J=6.8 Hz), 2.67 (2H, t, J=7.3 Hz), 2.28 (2H, t, J=7.3 Hz), 1.79-2.00 (1H, m), 1.43-1.75 (4H, m), 0.95 (6H, d, J=6.8 Hz).

### Synthesis of ENMD-1556: 5-[(3-methyl-butyrylamino)-methyl]-isoxazole-3-carboxylic acid (6-amino-hexyl)-amide

Following the general schemes provided, 5-bromomethyl-isoxazole-3-carboxylic acid was converted to an azide, saponified, coupled to mono-N-Boc-diaminohexane, reduced to the amine, coupled to 3-methylbutanoic acid, and deprotected.

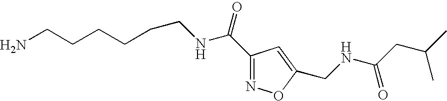

LCMS m/z 325(MH⁺). ¹H NMR (400 MHz, methanol-d4) δ ppm 6.58 (1H, s), 4.53 (2H, s), 3.37 (2H, t, J=7.1 Hz), 2.93 (2H, t, J=7.5 Hz), 1.98-2.17 (3H, m), 1.59-1.77 (4H, m), 1.35-1.52 (4H, m), 0.95 (6H, d, J=6.2 Hz).

### Synthesis of ENMD-1557: 5-[(2-Cyclohexyl-acetylamino)-methyl]-isoxazole-3-carboxylic acid (6-amino-hexyl)-amide

Following the general schemes provided, 5-bromomethyl-isoxazole-3-carboxylic acid was converted to an azide, saponified, coupled to mono-N-Boc-diaminohexane, reduced to the amine, coupled to cyclohexylacetic acid, and deprotected.

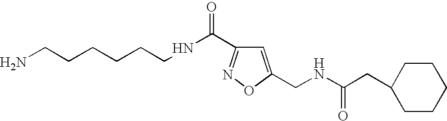

LCMS m/z 365(MH⁺). ¹H NMR (400 MHz, MeOD) δ ppm 6.56 (1H, s), 4.53 (2H, s), 3.37 (2H, t, J=7.1 Hz), 2.85-2.99 (2H, m), 2.12 (2H, d, J=7.3 Hz), 1.56-1.85 (10H, m), 1.37-1.54 (4H, m), 1.10-1.36 (3H, m), 0.85-1.07 (2H, m).

### Synthesis of ENMD-1558: 5-[(5-amino-pentanoylamino)-methyl]-isoxazole-3-carboxylic acid cyclohexylmethyl-amide

Following the general schemes provided, 5-bromomethyl-isoxazole-3-carboxylic acid was converted to an azide, saponified, coupled to cyclohexylmethylamine, reduced to the amine, coupled to N-Boc-aminopentanoic acid, and deprotected.

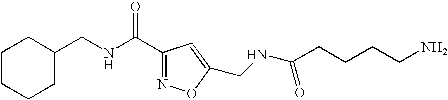

LCMS m/z 337 (MH⁺). ¹H NMR (400 MHz, MeOD) δ ppm 6.56 (1H, s), 4.53 (2H, s), 3.19 (2H, d, J=6.8 Hz), 2.70 (2H, t, J=7.1 Hz), 2.28 (2H, t, J=7.3 Hz), 1.46-1.84 (9H, m), 1.11-1.42 (4H, m), 0.86-1.08 (2H, m).

### Example 15

### General Schemes for Synthesis of Thiazoles

General Procedure for Capping of Amines:

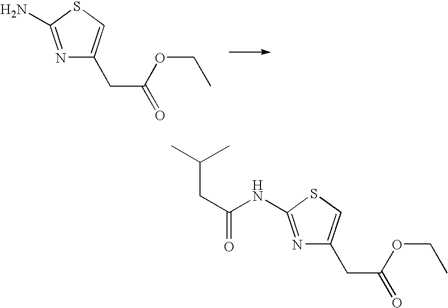

Same general procedure for capping conditions using HOAt and EDC as described for morpholines.

Material used: (2-amino-thiazol-4-yl)-acetic acid ethyl ester (160 mg, 0.86 mmol), HOAt (131 mg, 0.95 mmol, 1.1 eq.), EDC.HCl (185 mg, 0.95 mmol, 1.1 eq.), isovaleric acid (88 mg, 0.86 mmol, 1.0 eq.) in DMF (5 ml). 149 mg (64%) of product isolated after column chromatography over silica eluted with DCM then from 1:50 to 5:45 MeOH: DCM. LCMS m/z 271 (MH⁺).

General Procedure for Saponification:

See general procedure for hydrolysis of methyl ester with LiOH.H₂O as described for diaryls.

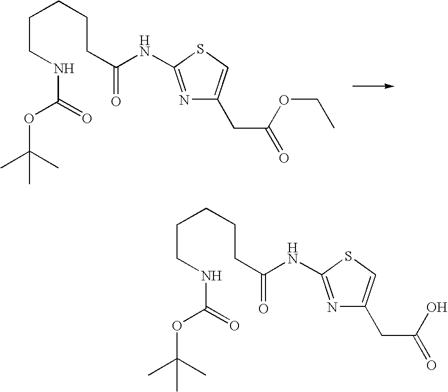

Material used: [2-(3-Methyl-butyrylamino)-thiazol-4-yl]-acetic acid ethyl ester (1.077 g, 2.70 mmol), LiOH.H₂O (0.68 g, 16.2 mmol, 6 eq.), THF (10 ml), water (10 ml) to yield 0.893 g (89%) of product. LCMS m/z 372 (MH⁺).

### Synthesis of ENMD-1561: N-{4-[(5-Amino-pentylcarbamoyl)-methyl]-thiazol-2-yl}-3-methyl-butyramide hydrochloride

Using the general schemes described, (2-amino-thiazol-4-yl)acetic acid ethyl ester was first coupled to 3-methylbutanoic acid using EDC, saponified, second coupled to mono-N-boc-diaminopentane, deprotected, and precipitated as the HCl salt.

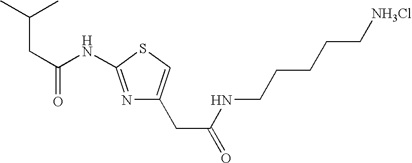

LCMS m/z 327.36 (MH⁺). ¹H-NMR 400 mHz d ppm 7.19 (1H, s), 3.78 (2H, s), 3.24 (2H, t, J=6.8 Hz), 2.93 (2H, t, J=7.7 Hz), 2.50 (2H, d, J=7.1 Hz), 2.14-2.28 (1H, m), 1.64-1.74 (2H, m), 1.52-1.64 (2H, m), 1.38-1.49 (2H, m), 1.02 (6H, d, J=6.6 Hz)

### Synthesis of ENMD-1549: N-{4-[(5-Amino-pentylcarbamoyl)-methyl]-thiazol-2-yl}-3-methyl-butyramide

Synthesis as for ENMD-1561, but prepared as the free base.

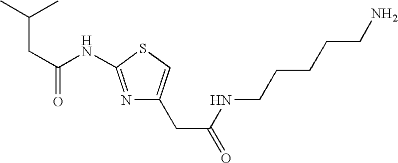

### Synthesis of ENMD-1554: 6-amino-hexanoic acid [4-(isobutylcarbamoyl-methyl)-thiazol-2-yl]-amide

Using the general schemes described, (2-amino-thiazol-4-yl)acetic acid ethyl ester was first coupled to N-Boc-aminohexanoic acid using EDC, saponified, second coupled to isobutylamine, and deprotected.

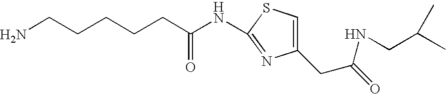

LCMS m/z 327 (MH⁺). ¹H NMR (400 MHz, MeOD) δ ppm 7.18 (1H, s), 3.78 (2H, s), 3.04 (2H, d, J=6.9 Hz), 2.87-3.00 (2H, m), 2.68 (2H, t, J=7.1 Hz), 1.64-1.89 (5H, m), 1.39-1.58 (2H, m), 0.92 (6H, d, J=6.9 Hz).

### Synthesis of ENMD-1559: N-(5-amino-pentyl)-2-[2-(2-cyclohexyl-acetyl-amino)-thiazol-4-yl]-acetamide

Using the general schemes described, (2-amino-thiazol-4-yl)acetic acid ethyl ester was first coupled to 2-cyclohexylacetic acid using EDC, saponified, second coupled to mono-N-boc-diaminopentane, and deprotected.

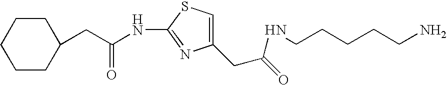

LCMS m/z 367 (MH⁺). ¹H NMR (400 MHz, methanol-d4) δ ppm 7.14 (1H, s), 3.75 (2H, s), 3.24 (2H, t, J=6.8 Hz), 2.93 (2H, t, J=7.3 Hz), 2.47 (2H, d, J=6.8 Hz), 1.82-1.96 (1H, m), 1.63-1.82 (7H, m), 1.51-1.63 (2H, m), 1.39-1.49 (2H, m), 1.17-1.38 (3H, m), 0.98-1.14 (2H, m).

### Synthesis of ENMD-1560: 6-amino-hexanoic acid {4-[(cyclohexylmethyl-carbamoyl)-methyl]-thiazol-2-yl}-amide

Using the general schemes described, (2-amino-thiazol-4-yl)acetic acid ethyl ester was first coupled to N-Boc-aminohexanoic acid using EDC, saponified, second coupled to cyclohexylmethylamine, and deprotected.

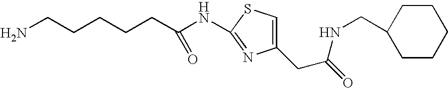

LCMS m/z 367 (MH⁺). ¹H NMR (400 MHz, DMSO-d₆) δ ppm 7.87 (1H, t, J=5.6 Hz), 6.82 (1H, s), 3.00-3.71 (4H, m), 2.76-3.02 (2H, m), 1.91-2.46 (1H, m), 1.01-1.81(15H, m), 0.61-0.97 (2H, m).

Benzimidazolones, azetidines, sultams, bicyclic amides, triazoles, pyrazines, pyrroles, pyridines, phenyls (diaminophenyls, hydroquinones or p-hydroxyphenols, phenyldicarboxylic acids, hydroxybenzoates, alkylbenzoates, carboxyanilines), alkanes, and alkynes were prepared using the using the illustrated synthetic schemes. Coupling conditions and synthetic and purification strategies were based on those shown above, using coupling reagents well known to those skilled in the art including CDI, EDC, and DCC. Side chain amines or other reactive groups were usually protected with t-Boc or Cbz or other appropriate protecting groups and were removed using standard conditions as shown or as described in the references.

### Example 16

### General Scheme for Synthesis of Azetidines

Using t-Boc protected azetidine, the first side chain was introduced by amine coupling with the appropriate acid chloride. The amide was deprotected with TFA followed by final coupling with a second acid chloride.

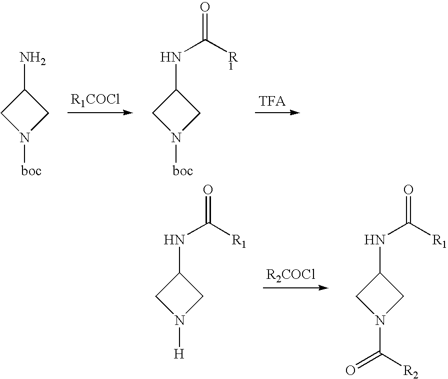

### Synthesis of ENMD-1513: N-[1-(2-cyclohexylacetyl)azetidin-3-yl]-6-aminohexanamide

Target was prepared by coupling 6-CBz-aminocaproic acid chloride with BOC protected azetidine, Boc removal with TFA and the second coupling using 2-cyclohexyl acetyl chloride. CBz deprotection using catalytic hydrogenation gave target.

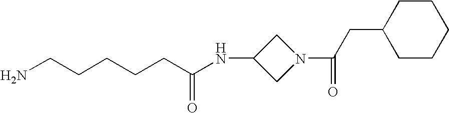

### Synthesis of ENMD-1514: 2-cyclohexylacetamid-N-(6-amino-1-hexanoyl azetidin-3-yl)

Target was prepared as ENMD-1513 except 2-cyclohexyl acetyl chloride was coupled to BOC protected azetidine and the second coupling was accomplished using 6-CBz-amino-caproic acid chloride.

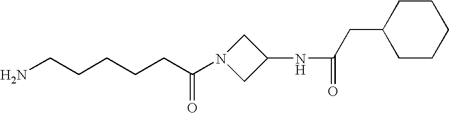

### Synthesis of ENMD-1515: N-[1-(3-methylbutanoyl)azetidin-3-yl]-6-aminohexanamide

Target was prepared using the same scheme as ENMD-1513 except isovaleric chloride was used as the second coupling reagent.

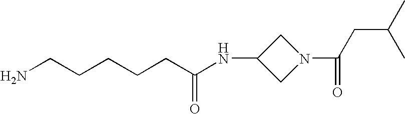

### Synthesis of ENMD-1516: N-(6-amino-1-hexanoylazetidin3-yl)-3-methylbutanamide

Target was prepared as ENMD-1513 except isovaleric chloride was the first coupling reagent and 6-CBz-amino-caproic acid chloride was the second coupling reagent.

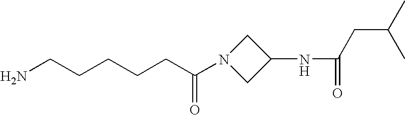

### Example 17

### General Scheme for Synthesis of Benzimidazolones

The side chains were introduced using potassium carbonate and the appropriate alkyl halide, followed by the second coupling with sodium hydride and the appropriate alkyl halide.

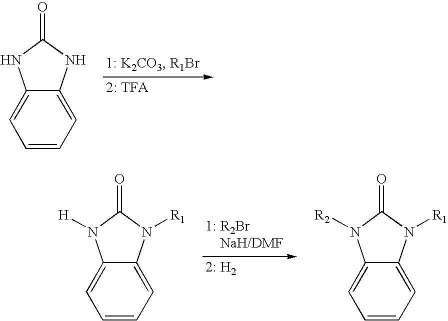

### Synthesis of ENMD-1517: 1-(6-aminohexyl)-3-[(3-methyl)butyl]benzimidazol(2)-one

Target was prepared by coupling N-Boc-benzimidazolone with isopentyl bromide and potassium carbonate, removal of the Boc protecting group with TFA and coupling with 6-CBz-aminohexyl bromide. Final deprotection of CBz by catalytic hydrogenation yielded ENMD-1517.

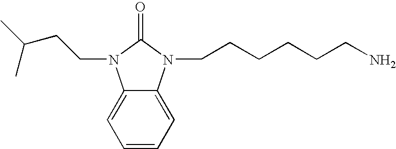

### Synthesis of ENMD-1518: 1-(6-aminohexyl)-3-[(2-cyclohexyl)ethyl]benzimidazol-(2)-one

Target was prepared using the same conditions as ENMD-1517 except 1-bromo-2-cyclohexyl ethane was used instead of isopentyl bromide.

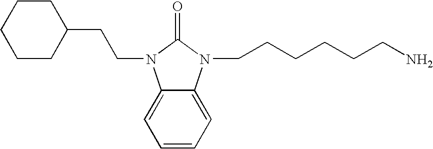

### Synthesis of ENMD-1563: 1-[4-(isopropylamine)-phenethyl]-3-isopentyl-1H-benzo[d]imidazole-2(3H)-one

Target was prepared during the attempted Pd/C reduction of the nitro precursor of ENMD-1573 in acetone.

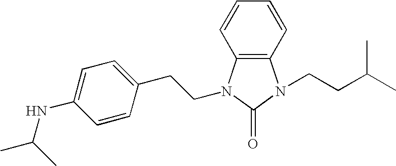

### Synthesis of ENMD-1564: 1-[4-(isopropylamine)-phenethyl]-3-(2-cyclo-hexylethyl)-1H-benzo[d]imidazole-2(3H)-one

Target was prepared using the attempted Pd/C reduction of the nitro precursor of ENMD-1574 in acetone.

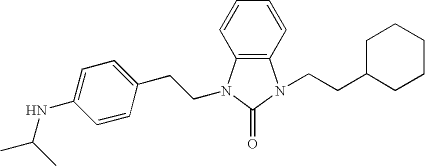

### Synthesis of ENMD-1573: 1-(4-aminophenethyl)-3-isopentyl-1H-benzo[d]imidazole-2(3H)-one

Target was prepared by coupling benzimidazolone with 2-(4-nitrophenylethyl)-bromide in the prescence of potassium carbonate. The second coupling was done using sodium hydride with isopentyl bromide and the nitro group was reduced using Pd/C in EtOH to give ENMD-1573.

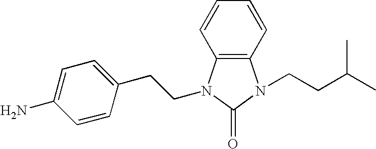

### Synthesis of ENMD-1574: 1-(4-aminophenethyl)-3-(2-cyclohexylethyl)-1H-benzo[d]imidazole-2(3H)-one

Target was prepared using the same conditions as ENMD-1573 except 1-bromo-2-cyclohexyl ethane was used for the second coupling.

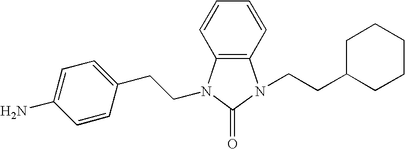

### Example 18

### General Scheme for Synthesis of Triazoles

Triazoles were prepared by a [3+2] Cycloaddition reaction using catalysis with Cu powder, followed by deprotection via catalytic hydrogenation as described.

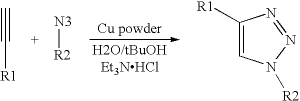

### Synthesis of ENMD-1519: 1-(6-aminohexyl)-4-(3-methylbutyl)-1,2,3-triazole

Target was prepared by Cu catalyzed (10 mol % catalyst) [3+2] cycloaddition between isohept-1-yne and 1-azido-6-(Cbz-amino)hexane followed by deprotection of the CBz.

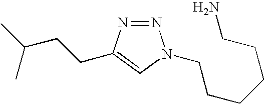

### Synthesis of ENMD-1520: 1-(6-aminohexyl)-4-(2-cyclohexylethyl)-1,2,3-triazole

Target was prepared by Cu catalyzed (10 mol % catalyst) [3+2] cycloaddition between 4-cyclohexylbut-1-yne and 1-azido-6-(Cbz-amino)hexane followed by deprotection of CBz.

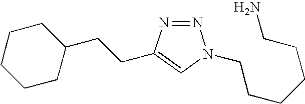

### Synthesis of ENMD-1542: 1-cyclohexylmethyl-4-(6-aminohexyl)1,2,3-triazole

Target was prepared by Cu catalyzed (10 mol % catalyst) [3+2] cycloaddition between 8-(N-Cbz-amino)oct-1-yne and azido-methylene-cyclohexane followed by deprotection of CBz.

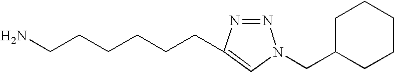

### Synthesis of ENMD-1544: 1-(2-methylpropyl)4-(6-aminohexyl)-1,2,3-triazole

Target was prepared by Cu catalyzed (10 mol % catalyst) [3+2] cycloaddition between between 8-(N-Cbz-amino)oct-1-yne and azido-isobutane followed by deprotection of the CBz.

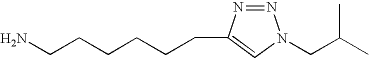

### Example 19

### General Scheme for Synthesis of Sultams

Sultams were prepared via an intramolecular cyclization reaction with Cl₂ following methods such as described in J. Chem. Soc. Perkin Trans. 1, (2001) pages 2022-2034 and J. Med. Chem., (2004) vol 47, pages 2981-2983).

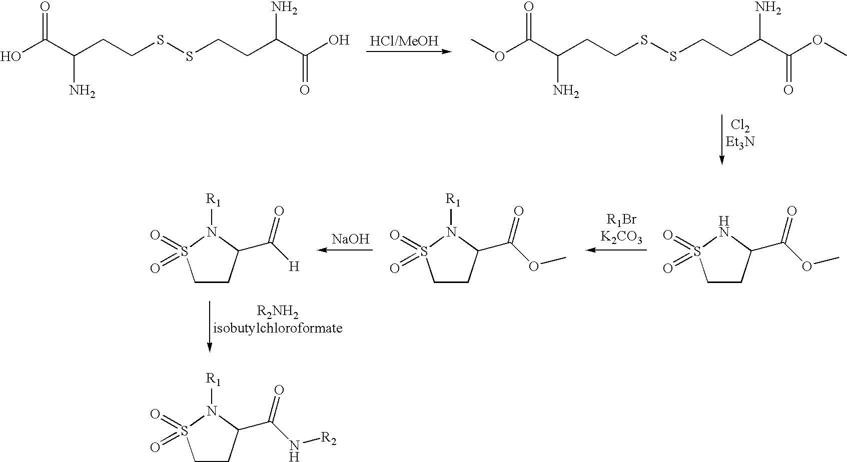

### Synthesis of ENMD-1539: N-(isoamyl)isothiasolidine-1,1-dioxide-3-carboxylic acid-6-aminohexylamide

Target was prepared by esterification of the disulfide followed by cyclization to the sultam with Cl₂. The nitrogen was alkylated with isopentyl bromide, ester hydrolysis and final coupling with 6-CBz-amino-1-aminohexane in the presence of isobutylchloroformate. Removal of the CBz group was accomplished by catalytic hydrogenation as described.

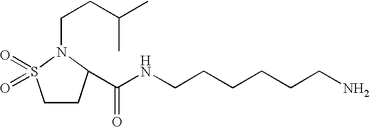

### Synthesis of ENMD-1545: N-(6-aminohexyl)isothiazolidine-1,1-dioxide-3-carboxylic acid (2-cyclohexyl)ethylamide

Target was prepared as ENMD-1539 except with 6-CBz-amino-1-bromohexane was the first coupling reagent and the second coupling was accomplished with 2-cyclohexyl-1-aminoethane. CBz was removed by catalytic hydrogenation.

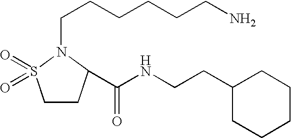

### Synthesis of ENMD-1546: N-(6-aminohexyl)isothiazolidine-1,1-dioxide-3-carboxylic acid isoamylamide

Target was prepared as ENMD-1545 except isopentyl amine was the second coupling reagent. CBz was removed by catalytic hydrogenation.

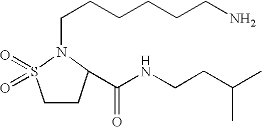

### Synthesis of ENMD-1547: N-(2-cyclohexyl)ethylisothiasolidine-1,1-dioxide-3-carboxylic acid-6-aminohexylamide

Target was prepared as ENMD-1539 except 2-cyclohexyl ethyl bromide was the first coupling reagent. CBz was removed by catalytic hydrogenation.

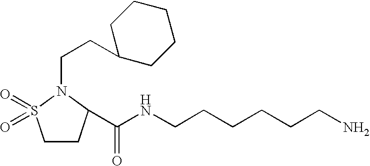

### Example 20

### General Scheme for Synthesis of Pyrazines

Pyrazines were prepared by coupling commercially available chloropyrazine with the appropriate alkyl amine under basic conditions. The resulting ester was hydrolyzed with LiOH and the acid was converted to an amide using EDCI activation.

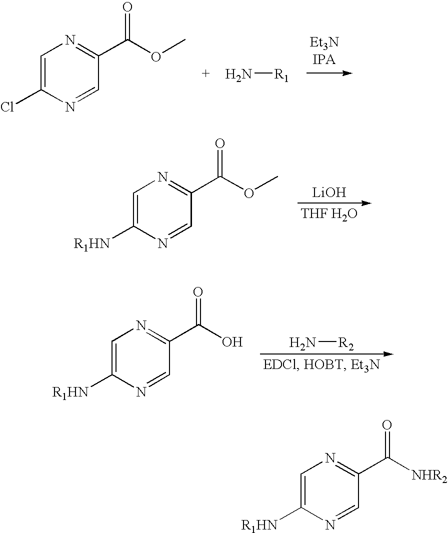

### Synthesis of ENMD-1571: 4-aminobutyl-6-cyclohexanemethylamino-2-pyrazine amide

Target was prepared by first coupling cyclohexylmethyl amine followed by ester hydrolysis and amide formation with N-Boc-1,4-diaminobutane. Deprotection was accomplished with TFA.

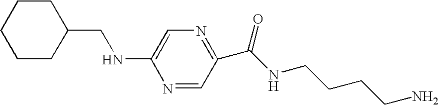

### Synthesis of ENMD-1572: 4-(piperidinethyl)-6-cyclohexanemethylamino-2-pyrazine amide

Target was prepared as ENMD-1571 except 1-Boc-piperidine-4-ethylamine was used for the amide coupling.

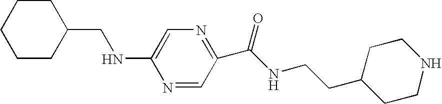

### Synthesis of ENMD-1775: cyclohexylmethyl-6-(5-aminopentaneamino)-2-pyrazine amide

Target was prepared by first coupling N-Boc-1,5-diaminopentane, followed by ester hydrolysis. The amide was prepared by coupling cyclohexyl methyl amine to the acid with EDCI. Boc was removed with TFA.

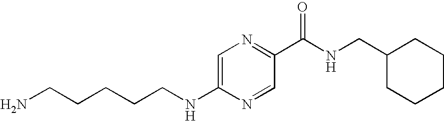

### Synthesis of ENMD-1778: cyclohexylethyl-6-(4-aminoethylpiperdine)-2-pyrazine amide

Target was prepared by coupling 4-N-Boc-piperidine ethylamine, ester hydrolysis and then amide formation with cyclohexylmethyl amine.

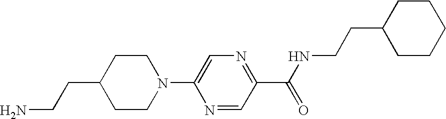

### Example 21

### General Scheme for Synthesis of Pyrroles

Commercially available 2-carboxypyrrole was esterified under acidic conditions to yield a methyl ester. The pyrrole was alkylated using potassium carbonate, followed by ester hydrolysis to give the acid. The acid was converted to an amide using oxalyl chloride and the appropriate amine.

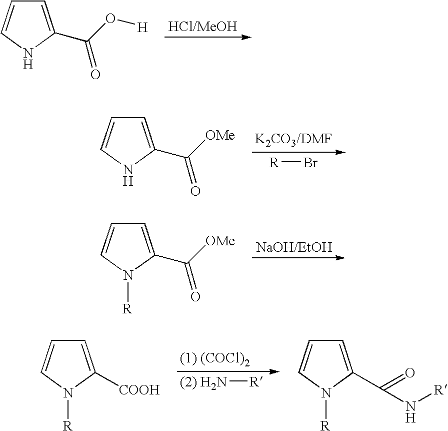

### Synthesis of ENMD-1537: N-(6-aminohexyl)-1-(2-methylpropyl)-2-pyrrolecarboxamide

Target was prepared by converting 2-carboxyacid pyrrole to the methyl ester, and alkylating the N with isobutyl bromide. The ester was hydrolyzed and the resulting acid was converted to an amide using oxalyl chloride and N-Boc-1,6-diaminohexane. Boc deprotection yielded the target.

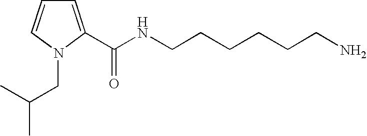

### Synthesis of ENMD-1540: N-(6-aminohexyl)-1-(2-cyclohexylmethyl)-2-pyrrolecarboxamide

Target was prepared as in ENMD-1537 except cyclohexyl methyl amine was used in the first coupling reaction.

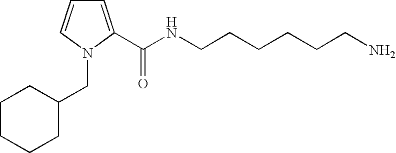

### Synthesis of ENMD-1569: 1-(6-aminohexyl)-2-[N′-isobutyl]-pyrrole carboxamide

Target was prepared as in ENMD-1537 except the first coupling used 6-tosyl-1-N-Boc-aminohexane in the first coupling reaction and isobutyl amine was used for the amide formation.

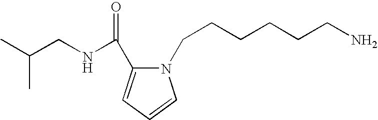

### Synthesis of ENMD-1570: 1-(6-aminohexyl)-2-[N′-cyclohexylmethyl)]-pyrrole carboxamide

Target was prepared as in ENMD-1537 except 6-tosyl-1-N-Boc-aminohexane was used in the first coupling reaction and cyclohexyl methyl amine was used in the second coupling reaction.

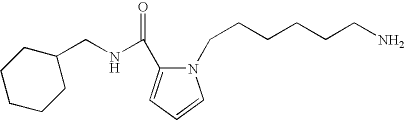

### Example 22

### General Scheme for Synthesis of Pyridines

Pyridines were prepared by converting the acid chloride to an amide followed by heating with the appropriate amine neat at 120° C. to displace the chloride.

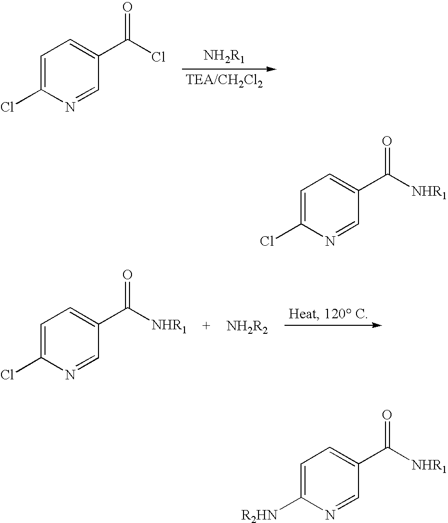

### Synthesis of ENMD-1538: 5-(cyclohexylmethylamino)-3-(4-aminobutyl)-nicotinoylamide

Following the general schemes provided, 6-chloropyridine-3-carbonyl chloride was reacted with N-Boc-1,4-diaminobutane in TEA and CH₂Cl₂. The second coupling was accomplished by heating with cyclohexylmethylamine (neat) at 120° C., and removing the Boc group to give ENMD-1538.

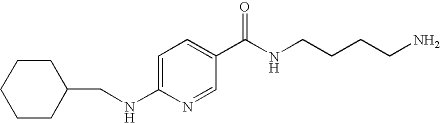

### Synthesis of ENMD-1541: 5-(isobutylamino)-3-(4′-aminobutyl)-nicotinoyl amide

Following the general schemes provided, 6-chloropyridine-3-carbonyl chloride was reacted with N-Boc-1,4-diaminobutane in TEA and CH₂Cl₂. The second coupling was accomplished by heating with isobutylamine (neat) at 120° C., and removing the Boc group to give ENMD-1541.

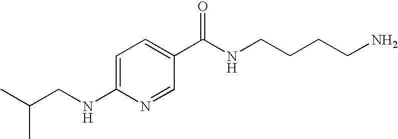

### Synthesis of ENMD-1543: 5-(4-aminobutylamine)-3-(cyclohexylmethyl)-nicotinoyl amide

Following the general schemes provided, 6-chloropyridine-3-carbonyl chloride was reacted with cyclohexylmethylamine in TEA and CH₂Cl₂. The second coupling was accomplished by heating with N-Boc-1,4-diaminobutane (neat) at 120° C., and removing the Boc group to give ENMD-1543.

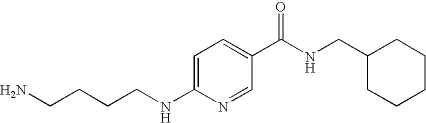

### Synthesis of ENMD-1562: 6-(4-aminobutylamine)-3-(isobutyl)-nicotinoyl amide

Following the general schemes provided, 6-chloropyridine-3-carbonyl chloride was reacted with isobutylamine in TEA and CH₂Cl₂. The second coupling was accomplished by heating with N-Boc-1,4-diaminobutane (neat) at 120° C., and removing the Boc group to give ENMD-1562.

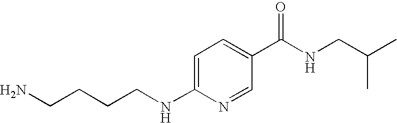

### Example 23

### General Schemes for Preparation of Amides

Amide analogs were prepared by coupling the appropriate amines and acids. In some cases coupling agents including DCC, CDI, or EDCI were used, while in some cases acids were activated as acid chlorides. The amine side chains and other reactive groups were protected with Cbz or tBoc or as esters, and protection groups were removed after the coupling reaction/s using standard conditions known to those skilled in the art. In one set of examples shown in the following scheme, target compounds were synthesized by coupling methyl 4-aminobenzoate with the appropriate acid side chain with either DCC/HOBT or CDI, hydrolyzing the ester with base, and coupling the second side chain with DCC/HOBT.

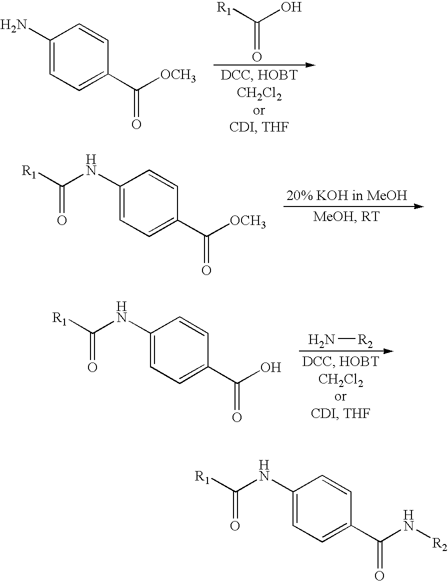

### Synthesis of ENMD-1511: N-(4-aminobutyl)-4-(cyclohexanecarboxamido)-benzamide hydrochloride

Methyl 4-aminobenzoate was coupled with cyclohexanecarboxylic acid using DCC/HOBT in CH₂Cl₂, hydrolyzed with 20% KOH in MeOH, then coupled with N-Cbz-1,4-diaminobutane hydrochloride using DCC/HOBT in CH₂Cl₂. Cbz was removed with Pd—C (10%) at 50 psi of H₂(g) and then converted to the hydrochloride salt using HCl (g) in MeOH.

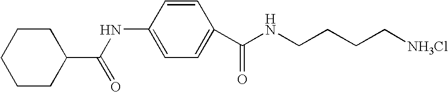

### Synthesis of ENMD-1568: 4-(5-aminopentanamide)-N-(cyclohexylmethyl)-benzamide

Methyl 4-aminobenzoate was coupled with 5-(Cbz-amino)pentanoic acid using CDI in THF, hydrolyzed with 20% KOH in MeOH, and then coupled with cyclohexylmethyl amine using CDI in THF. Cbz protection group was then removed with Pd—C (10%) at 50 psi of H₂(g).

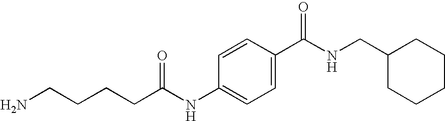

### Synthesis of ENMD-1391: N-(4-(3-methylbutanamido)phenyl)-6-aminohexanamide

Synthesis by the general methods shown for amide couplings. Cbz-6-aminocaproic acid was coupled with p-phenylendiamine (1,4 diaminobenzene) using DCC/HOBT in dichloromethane. Second coupling with isovaleryl chloride was performed in pyridine to give 70% yield. Deprotection of Cbz with Pd—C 10% in methanol at 50 psi of H₂ gas gave final product.

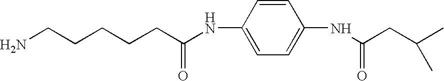

### Synthesis of ENMD-1397: N-(4-(3-methylbutanamido)phenyl)-4-aminobutyl-amide hydrobromide

Cbz-4-aminobutyric acid was coupled with p-phenylendiamine using DCC/HOBT in dichloromethane. Second coupling with isovaleric acid was performed using DCC/HOBT in dichloromethane to give 70% yield. Deprotection of Cbz with HBr/HOAc gave final product.

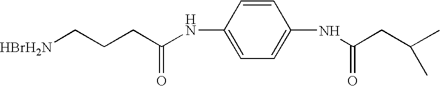

### Synthesis of ENMD-1393: N-(2-(3-methylbutanamido)ethyl)-6-aminohexan-amide hydrobromide

Isovaleryl chloride was coupled with mono-Cbz-1,2-diaminoethane hydrochloride in pyridine. The Cbz-group was deprotected using HBr/HOAc and then second coupling with Cbz-6-aminohexanoic acid was performed using CDI in THF to give 70% yield. Deprotection of Cbz with HBr/HOAc gave final product.

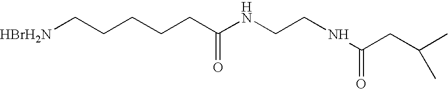

### Synthesis of ENMD-1416: N-(2-(2-(4-aminophenyl)acetamido)ethyl)-3-methylbutan-amide hydrochloride

Isovaleric acid was coupled with mono-Cbz-1,2-diaminoethane hydrochloride using CDI in THF. The Cbz protecting group was deprotected using HBr/HOAc and then second coupling with t-Boc-4-aminophenylacetic acid was performed using CDI in THF to give 60% yield. Deprotection of t-Boc with TFA in dichloromethane gave final product.

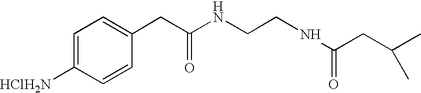

### Synthesis of ENMD-1417: N-(2-(2-cyclohexylacetamido)ethyl)-6-aminohexan-amide

Cyclohexylacetic acid was coupled with Cbz-protected-1,2-diaminoethane hydrochloride using CDI in THF. The Cbz-group was deprotected using HBr/HOAc and then second coupling with CBZ-6-amino caproic acid was performed using CDI in THF to give 60% yield. Deprotection of Cbz with Pd—C 10% in methanol at 50 psi of H2 gas gave final product.

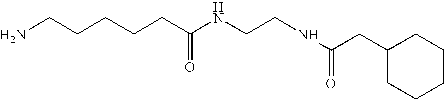

### Synthesis of ENMD-1418: N-(2-(2-cyclohexylacetamido)ethyl)-6-aminohexan-amide

Cyclohexylacetic acid was coupled with Cbz-protected-1,2-diaminoethane hydrochloride using CDI in THF. The Cbz-group was deprotected using HBr/HOAc and then second coupling with t-BOC-4-aminophenylacetic acid was performed using CDI in THF to give 60% yield. Deprotection of t-Boc with TFA in dichloromethane gave final product.

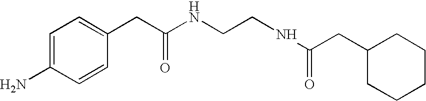

### Synthesis of ENMD-1504: N-isobutyl-3R-(6-aminohexanamido)-cyclopentane-1R-carboxamide

Synthesis by the general methods shown for amide couplings. (1R,3R)-N-Boc-1-aminocyclopentane-3-carboxylic acid was coupled with isobutylamine using carbonyldiimidazole (CDI) in THF. The second coupling with Cbz-6-aminocaproic acid was performed using CDI in THF to give 50% yield. Deprotection of Cbz with Pd—C 10% in methanol gave final product.

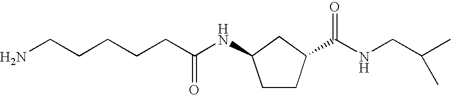

### Synthesis of ENMD-1505: N-cyclohexylmethyl-3S-(6-aminohexanamido)-cyclopentane-1R-carboxamide

(1R,3S)-N-Boc-1-aminocyclopentane-3-carboxylic acid was coupled with cyclohexanemethylamine using CDI in THF. The second coupling with Cbz-6-aminocaproic acid was performed using CDI in THF to give 50% yield. Deprotection of Cbz with Pd—C 10% in methanol gave final product.

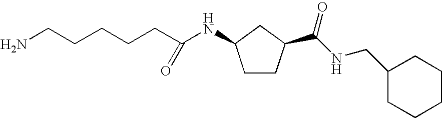

### Synthesis of ENMD-1536: N-isobutyl-3S-(6-aminohexanamido)-cyclopentane-1R-carboxamide

(1R,3S)-N-Boc-1-aminocyclopentane-3-carboxylic acid was coupled with isobutylamine using CDI in THF. The second coupling with (1R,3S)-1-aminocyclopentane-3-isobutylcarboxyamide and Cbz-6-aminocaproic acid was performed using CDI in THF to give 50% yield. Deprotection of Cbz with Pd—C 10% in methanol gave final product.

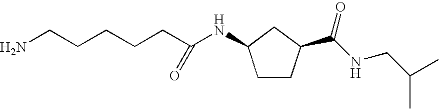

### Synthesis of ENMD-1766: N¹-(5-aminopentyl)-N-4-(cyclohexylmethyl)-terephthalamide

Mono-methyl-terephthalic acid was coupled with cyclohexylmethylamine using CDI in THF. The methyl ester was demethylated with Claisin alkali in MeOH and then secondnd coupling with N-Cbz-1,5-diaminopentane-HCl was performed using CDI in THF to give 50% yield. Deprotection of Cbz with H2/Pd—C 10% in MeOH gave final product.

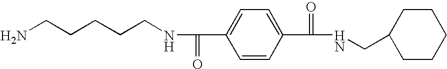

¹H NMR (300 MHz, DMSO-d₆) δ 8.64 (t, 1H, J=5.5 amide), 8.54 (t, 1H, J=5.5, amide), 7.9 (s, 4H, aromatic), 7.85 (s, 2H, broad), 3.34 (s, 2H), 3.30 (t, 2H, J=6.27), 3.13 (t, 2H, J=6.1), 2.78 (s, 2H), 1.78-1.48(m, 10H), 1.4 (q, 2H, J=7.0) 1.25-1.10 (m, 3H), 0.95 (2H, t J=7.57 Hz).

### Example 24

### General Schemes for Syntheses with Heterocyclic and Carbocyclic Amine Substituents

Methods described elsewhere for synthesis of amides were used. Examples were prepared with aromatic, saturated, carbocyclic, and/or heterocyclic linkers. For example, in one example of a target with a saturated heterocyclic core, the first coupling linked 1-Boc-piperazine with 2-cyclohexylacetic acid using CDI in THF. The Boc protecting group was removed with TFA in CH₂Cl₂ and converted to HCl salt with HCl (g) in MeOH. The resulting amine was then coupled with the appropriate side chains using DCC/HOBT in CH₂Cl₂. Removal of Boc protecting group with TFA in CH₂Cl₂, followed by conversion to HCl salt with HCl (g) in MeOH produced the desired products.

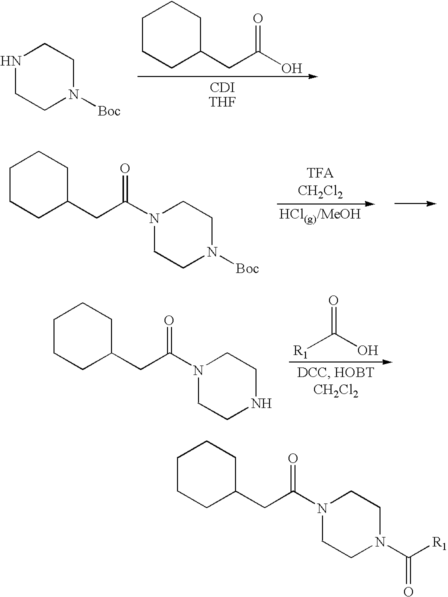

### Synthesis of ENMD-1768: (1R,3S)-N-(4-(2-cyclohexylacetamido)phenyl)-3-aminocyclopentanecarboxamide hydrochloride

Cyclohexylacetic acid was coupled with p-phenylendiamine using carbonyldiimidazole (CDI) in THF. The second coupling with (1R,3S)-N-Boc-1-aminocyclopentane-3-carboxylic acid was performed using DCC/HOBT in DMF to give 50% yield. Deprotection of t-Boc with TFA in dichloromethane and conversion to the hydrochloride using HCl in isopropyl alcohol gave final product.

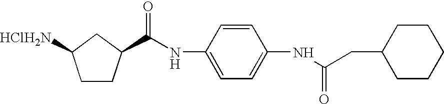

¹H NMR (300 MHz, DMSO-d₆) δ 8.64 (t, 1H, J=5.5 amide), 8.54 (t, 1H, J=5.5, amide), 7.9 (s, 4H, aromatic), 7.85 (s, 2H, broad), 3.34 (s, 2H), 3.30 (t, 2H, J=6.27), 3.13 (t, 2H, J=6.1), 2.78 (s, 2H), 1.78-1.48(m, 10H), 1.4 (q, 2H, J=7.0) 1.25-1.10 (m, 3H), 0.95 (2H, t J=7.57 Hz).

### Synthesis of ENMD-1770: N¹-2-cyclohexylethanoyl-N-4-piperidine-4-carbonyl-piperazine hydrochloride

1-Boc-piperazine was coupled with 2-cyclohexylacetic acid using CDI in THF. The Boc protecting group was removed with TFA in CH₂Cl₂ and converted to HCl salt with HCl in MeOH. The resulting amine was coupled with 1-Boc-isonipecotic acid using DCC/HOBT in CH₂Cl₂. Removal of Boc protecting group with TFA in CH₂Cl₂, followed by conversion to HCl salt with HCl in MeOH.

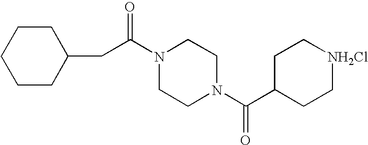

### Synthesis of ENMD-1771: N-(4-(2-cyclohexylacetamido)phenyl) piperidine-3-carboxamide hydrochloride

Cyclohexylacetic acid was coupled with p-phenylendiamine using CDI in THF. The second coupling with N-Boc-DL-nipecotic acid was performed using DCC/HOBT in DMF to give 54% yield. Deprotection of t-Boc with TFA in dichloromethane and conversion to the hydrochloride using HCl in isopropyl alcohol gave final product.

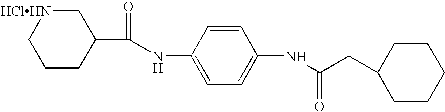

### Synthesis of ENMD-1772: N-(4-(2-cyclohexylacetamido)phenyl) piperidine-4-carboxamide hydrochloride

Cyclohexylacetic acid was coupled with p-phenylendiamine using CDI in THF. The second coupling with N-Boc-isonipecotic acid was performed using DCC/HOBT in DMF to give 58% yield. Deprotection of t-Boc with TFA in dichloromethane and conversion to the hydrochloride using HCl in isopropyl alcohol gave final product.

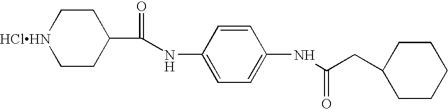

### Synthesis of ENMD-1773: N1-2-cyclohexylethanoyl-N-4-piperidine-3-carbonyl-piperazine hydrochloride

1-Boc-piperazine was coupled with 2-cyclohexylacetic acid using CDI in THF. The Boc protecting group was removed with TFA in CH₂Cl₂ and converted to HCl salt with HCl in MeOH. The resulting amine was coupled with N-Boc-DL-nipecotic acid using DCC/HOBT in CH₂Cl₂. Removal of Boc protecting group with TFA in CH₂Cl₂, followed by conversion to HCl salt with HCl in MeOH.

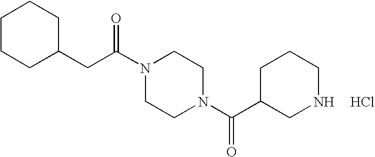

### Synthesis of ENMD-1774: N¹-2-cyclohexylethanoyl-N-4-cis-3-amino-cyclohexane-carbonyl piperazine hydrochloride

1-Boc-piperazine was coupled with 2-cyclohexylacetic acid using CDI in THF. The Boc protecting group was removed with TFA in CH₂Cl₂ and converted to HCl salt with HCl in MeOH. The resulting amine was coupled with cis-3-(Boc-amino)cyclohexane carboxylic acid using DCC/HOBT in CH₂Cl₂. Removal of Boc protecting group with TFA in CH₂Cl₂, followed by conversion to HCl salt with HCl in MeOH.

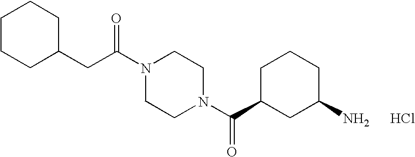

### Synthesis of ENMD-1779: N1-2-cyclohexylethanoyl-N-4-(1R,3R)-1-amino-cyclopentane-3-carbonyl piperazine hydrochloride

1-Boc-piperazine was coupled with 2-cyclohexylacetic acid using CDI in THF. The Boc protecting group was removed with TFA in CH₂Cl₂ and converted to HCl salt with HCl in i-PrOH. The resulting amine was coupled with (1R,3R)-N-Boc-1-aminocyclo pentane-3-carboxylic acid using DCC/HOBT in CH₂Cl₂. Removal of Boc protecting group with TFA in CH₂Cl₂, followed by conversion to HCl salt with HCl in MeOH.

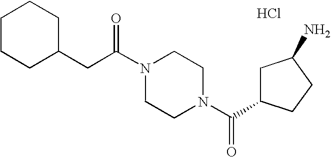

### Synthesis of ENMD-1780: N¹-2-cyclohexylethanoyl-N-4-morpholine-2-carbonyl-piperazine hydrochloride

1-Boc-piperazine was coupled with 2-cyclohexylacetic acid using CDI in THF. The Boc protecting group was removed with TFA in CH₂Cl₂ and converted to HCl salt with HCl (g) in MeOH. The resulting amine was coupled with (R,S)-Boc-2-carboxymorpholine using DCC/HOBT in CH₂Cl₂. Removal of Boc protecting group with TFA in CH₂Cl₂, followed by conversion to HCl salt with HCl (g) in MeOH.

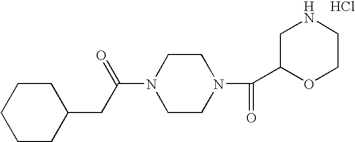

### Synthesis of ENMD-1781: N¹-2-cyclohexylethanoyl-N-4-cis-4-amino-cyclohexane-carbonyl piperazine hydrochloride

1-Boc-piperazine was coupled with 2-cyclohexylacetic acid using CDI in THF. The Boc protecting group was removed with TFA in CH₂Cl₂ and converted to HCl salt with HCl in MeOH. The resulting amine was coupled with cis-4-(Boc-amino)cyclohexane carboxylic acid using DCC/HOBT in CH₂Cl₂. Removal of Boc protecting group with TFA in CH₂Cl₂, followed by conversion to HCl salt with HCl in MeOH.

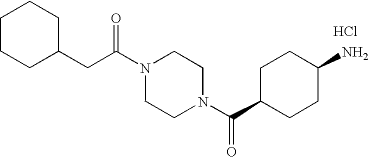

### Example 25

### General Schemes for Synthesis of Hydroxyphenyls

Ethers of alcohols and phenols can be prepared using standard methods which are known to those skilled in the art. Targets containing both ethers and amides were prepared by combinations of the schemes shown for preparation of ethers and for preparation of amides. For example, ENMD-1405 was prepared by alkylating methyl 4-hydroxybenzoate with the appropriate alkyl halide using K₂CO₃ in acetone under reflux, hydrolyzing the ester using either acid or base, and coupling the resulting acid with the second side chains with DCC/HOBT in CH₂Cl₂ to produce the amide. Amine side chains were protected with either t-Boc or Cbz which were removed after the coupling reactions using standard conditions.

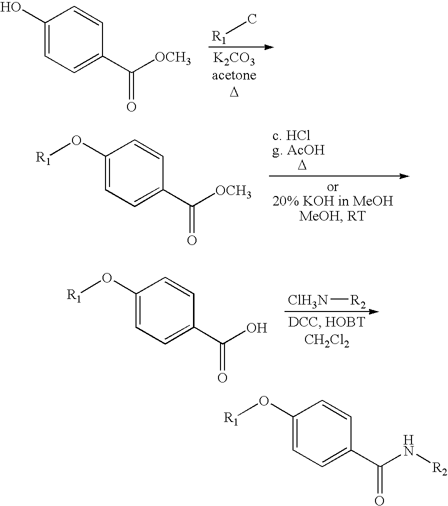

### Synthesis of ENMD-1405: N-(4-aminobutyl)-4-isobutoxy-benzamide

Methyl 4-hydroxybenzoate was alkylated with 1-iodo-2-methylpropane, and the ester was hydrolyzed with concentrated HCl in refluxing glacial acetic acid. The resulting acid was coupled with N-Cbz-1,4-diaminobutane hydrochloride using DCC/HOBT in CH₂Cl₂. Removal of Cbz with Pd—C (10%) in 2:1 CHCl₃:MeOH at 50 psi of H₂(g) gave ENMD-1405.

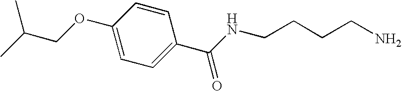

### Synthesis of ENMD-1406: N-(4-aminobutyl)-4-(cyclohexylmethoxy)-benzamide

Methyl 4-hydroxybenzoate was alkylated with (bromomethyl)cyclohexane using K₂CO₃ in acetone under reflux, and the ester was hydrolyzed with concentrated HCl in refluxing glacial acetic acid. The second side chain was introduced by coupling the resulting acid with N-Cbz-1,4-diaminobutane hydrochloride using DCC/HOBT in CH₂Cl₂. Removal of Cbz with Pd—C (10%) in 2:1 CHCl₃:MeOH at 50 psi of H₂(g) gave ENMD-1406.

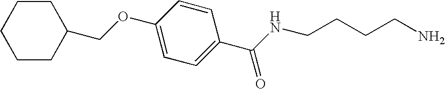

### Synthesis of ENMD-1408: N-(3-aminopropyl)-4-isobutoxy-benzamide

Methyl 4-hydroxybenzoate was alkylated with 1-iodo-2-methylpropane using K₂CO₃ in acetone under reflux, and the ester was hydrolyzed with concentrated HCl in refluxing glacial acetic acid. The second side chain was introduced by coupling the resulting acid with N-Cbz-1,3-diaminopropane hydrochloride using DCC/HOBT in CH₂Cl₂. Removal of Cbz with Pd—C (10%) in CHCl₃ at 50 psi of H₂ gave ENMD-1408.

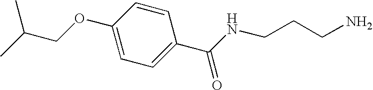

### Synthesis of ENMD-1409: N-(3-aminopropyl)-4-(cyclohexylmethoxy)-benzamide

Synthesis as ENMD-1408 except first alkylation was with bromomethyl-cyclohexane. Removal of Cbz with Pd—C (10%) in 4:1 CHCl₃:MeOH at 50 psi of H₂ gave ENMD-1409.

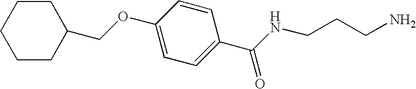

### Synthesis of ENMD-1410: 4-(3-aminopropoxy)-N-isobutyl-benzamide

Methyl 4-hydroxybenzoate was alkylated with Boc protected 3-bromopropyl amine using K₂CO₃ in acetone under reflux, and the ester was hydrolyzed with 20% KOH in MeOH. The second side chain was introduced by coupling the resulting acid with 2-methylpropyl amine using DCC/HOBT in CH₂Cl₂. Boc protection group was then removed with TFA in CH₂Cl₂ to give ENMD-1410.

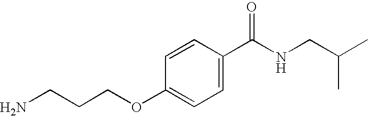

### Synthesis of ENMD-1411: 4-(3-aminopropoxy)-N-(cyclohexylmethyl)-benzamide

Synthesis as for ENMD-1410 except the second side chain was introduced by coupling the resulting acid with cyclohexyl-methylamine using DCC/HOBT in CH₂Cl₂.

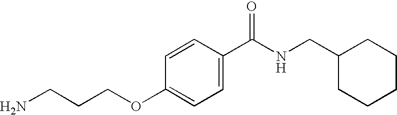

### Synthesis of ENMD-1485: N-(4-aminobenzyl)-4-(cyclohexylmethoxy)-benzamide hydrochloride

Methyl 4-hydroxybenzoate was alkylated with (bromomethyl)cyclohexane using K₂CO₃ in acetone under reflux, and the ester was hydrolyzed with concentrated HCl in refluxing glacial acetic acid. The second side chain was introduced by coupling the resulting acid with 4-(aminomethyl)-N-Boc-benzenamine using DCC/HOBT in CH₂Cl₂. Boc protection group was then removed with TFA in CH₂Cl₂ and converted to HCl salt with HCl in MeOH to give ENMD-1485.

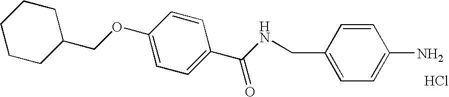

### Synthesis of ENMD-1566: 4-(4-(benzyloxy)phenoxy)butan-1-amine hydrochloride

Hydroquinone (p-hydroxyphenol) was reacted with benzyl bromide by refluxing with K₂CO₃ in acetone. The second coupling with 3-bromobutyl-(carbamic acid t-butyl ester) was performed using same procedure to give 60% yield. Deprotection of t-Boc with TFA in CH₂Cl₂ gave target.

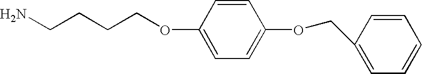

### Synthesis of ENMD-1567: 3-(4-(cyclohexylmethoxy)phenoxy)propan-1-amine hydrochloride

Hydroquinone was reacted with 1-bromomethylcyclohexane by refluxing with K₂CO₃ in acetone. The second coupling with 3-bromopropyl-(carbamic acid t-butyl ester) was performed using same procedure to give 54% yield. Deprotection of t-Boc with TFA in CH₂Cl₂ gave target.

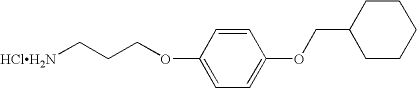

### Example 26

### Synthesis of ENMD-1509: N-(4-aminobutyl)-4-(2-cyclohexylethyl)-benzamide

4-formylbenzoic acid was coupled with N-Cbz-1,4-diaminobutane hydrochloride using DCC/HOBT in CH₂Cl₂. Wittig reaction of the aldehyde with cyclohexyl-methyl triphenyl phosphonium bromide followed by reduction of the alkene and removal of Cbz resulted in ENMD-1509.

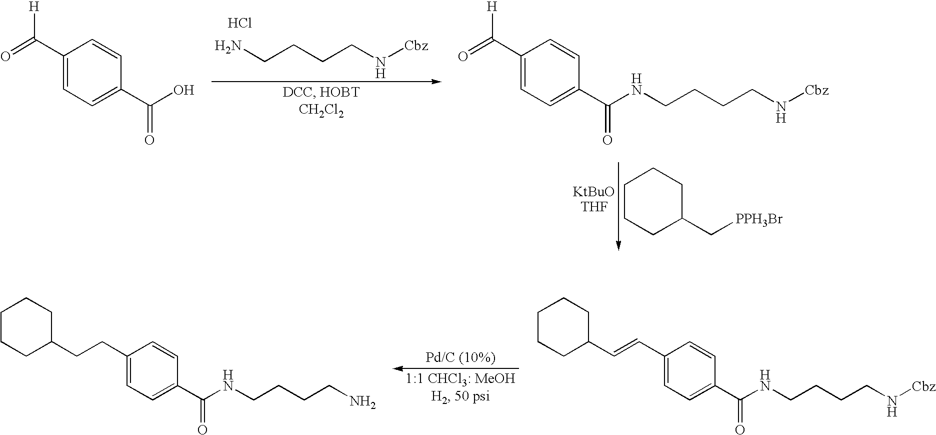

### Example 27

### Synthesis of ENMD-1535: N-cyclohexyl-2-(1-(4-(1,3-dioxoisoindolin-2-yl)butyl)-1H-imidazol-4-yl)acetamide

4-imidazolacetic acid-HCl was protected with trityl chloride in pyridine at 70° C. for 3 hr and coupled with cyclohexylmethylamine using CDI in THF. The trityl protecting group was removed by catalytic hydrogenation, and then reacted with NaH and N-(4-bromobutyl)-phthalamide in 3:1 THF:DMF to give the product.

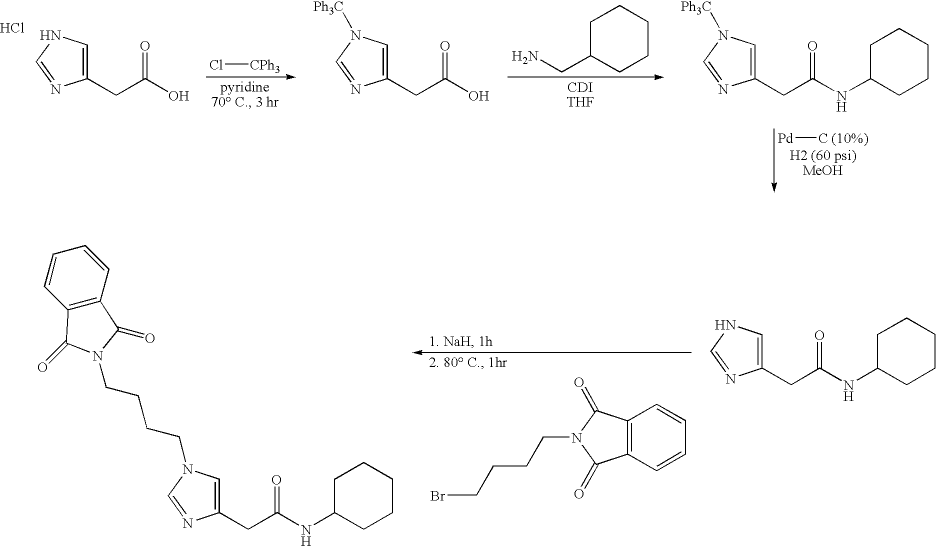

### Example 28

### Synthesis of ENMD-1552: N-(4-aminobutyl)-4-isopentyl-benzamide

Commercially available 4-(3-methylbut-3-enyl)benzoic acid was coupled to N-Cbz-1,4-diaminobutanehydrochloride and then reduced with Pd—C 10% H₂ (50 psi) to give ENMD-1552.

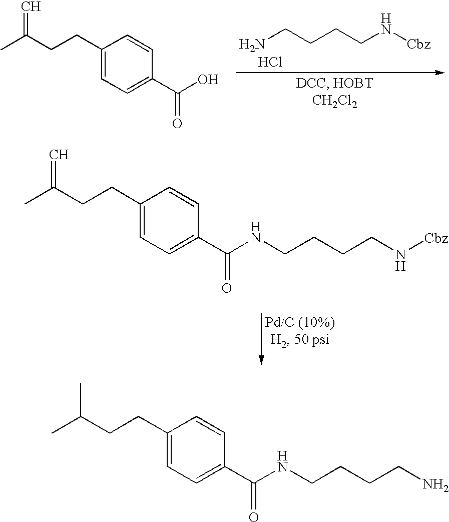

### Example 29

### General Scheme for Synthesis of Fused Bicyclic Amides

Using the commercially available t-Boc protected bicyclic ring amine shown, a mixed anhydride was prepared and coupled to the free amine. t-Boc was removed with TFA and the second coupling reaction was done using a mixed anhydride.

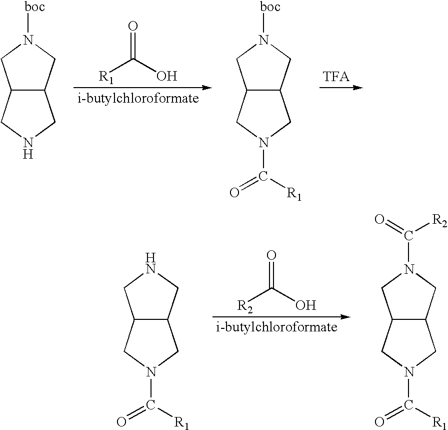

### Synthesis of ENMD-1763: 1-(2-(4-amino)phenyl)acetyl)hexahydropyrrolo-[3,4-c]-pyrrol-5(1H)-yl)-(3-methyl)butan-1-one

Boc-protected bicyclic amine was coupled to isovaleric acid chloride with triethyl amine. The Boc group was removed with TFA and 4-(Boc)aniline acetic acid was coupled using isobutyl chloroformate. Deprotection with TFA gave target.

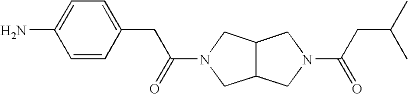

### Synthesis of ENMD-1764: 1-(2-(6-amino)hexanoyl)hexahydropyrrolo[3,4-c]-pyrrol-5(1H)-yl)-(3-methyl)butan-1-one

Target was prepared as ENMD-1763 except the second coupling used 6-CBz-aminocaproic acid.

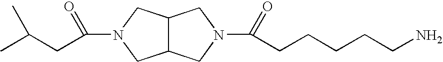

### Synthesis of ENMD-1776: 1-(2-(6-aminohexanoyl)hexahydropyrrolo[3,4-c]pyrrol-5-(1H)-yl)-2-cyclohexyl)ethan-1-one

Target was prepared as ENMD-1763 except the first coupling used 2-cyclohexyl acetic acid with isobutyl chloroformate. The second coupling was accomplished with 6-CBz-amino caproic acid in the presence of isobutyl chloroformate. Protecting groups were removed under the same conditions as above.

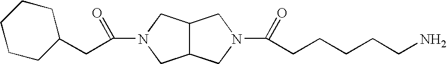

### Synthesis of ENMD-1777: 1-(2-(2-(4-aminophenyl)acetyl-hexahydro-pyrrolo[3,4-c]pyrrol-5-(1H)-yl)-2-cyclohexyl)ethan-1-one

Target was prepared as in ENMD-1776 except 4-(BOC)aniline acetic acid was used as the acid in the second amide coupling step.

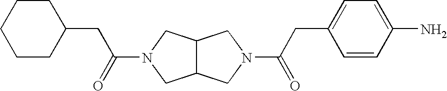

It should be understood that the foregoing relates only to preferred embodiments of the present invention, and that numerous modifications or alterations may be made therein without departing from the spirit and the scope of the invention as set forth in the appended claims.

Where specified enantiomers are shown or are chemically possible, both the R and S or the D and L enantiomers or the racemates or mixtures of the enantiomers in any ratio are envisioned by this invention.

1. A composition comprising a molecule;
wherein the molecule comprises a first component, a linker and a second component;
wherein the first component comprises a basic portion, a polar portion or a hydrogen-bonding portion;
wherein the second component comprises a hydrophobic moiety.

2. The composition of claim 1, wherein the first component comprises an alcohol, amine, acid, guanine, ester or amide functional groups.

3. The composition of claim 2, wherein the first component further comprises linear or branched, saturated or unsaturated carbocyclic rings.

4. The composition of claim 2, wherein the first component further comprises linear or branched, saturated or unsaturated heterocyclic rings.

5. The composition of claim 2, wherein the first component comprises a chemical moiety that structurally, spatially, chemically, or electronically mimics lysine.

6. The composition of claim 1, wherein the second component comprises a substituted or unsubstituted straight or branched aliphatic.

7. The composition of claim 1, wherein the second component further comprises saturated carbocyclic rings, unsaturated carbocyclic rings, saturated heteroatom-containing rings or unsaturated heteroatom-containing rings.

8. The composition of claim 1, wherein the second component comprises a chemical moiety that stereochemically mimics leucine.

9. The composition of claim 1, wherein the linker comprises a chemical moiety which structurally, spatially, chemically, or electronically mimics the spacing provided by the Ile and Gly residues in LIGK.

10. The composition of claim 1, wherein the linker comprises saturated aromatic ring systems, unsaturated aromatic ring systems, aromatic ring systems, linear unsaturated hydrocarbon chains, branched unsaturated hydrocarbon chains, linear saturated hydrocarbon chains, branched saturated hydrocarbon chains, sugars, nucleotides or nucleosides, single ring unsaturated carbocycles, single ring saturated carbocycles, multiple ring unsaturated carbocycles, multiple ring saturated carbocycles, single ring unsaturated heterocycles, single ring saturated heterocycles, multiple ring unsaturated heterocycles, multiple ring saturated heterocycles, heteroatoms, halides, nitrogen, oxygen, sulfur, silicon, selenium, or phosphorous.

11. The composition of claim 1, wherein the linker is non-cyclic, wherein the terminal R groups are bound to any position on the linker, wherein the linkers have heteroatom-containing substituent groups, or wherein the linkers can have aliphatic groups other than simple linear or branched hydrocarbon chains.

12. The composition of claim 11, wherein the heteroatom-containing substituent groups comprise imidazoles, aminos, arginyls, aminophenyls, pyridyls, thiols, alcohols, acids, esters, halides or amides.

13. The composition of claim 1, wherein the linker comprises substituted or unsubstituted phenyls, benzyls, saturated or unsaturated branched or linear hydrocarbons (including alkanes, alkenes, or alkylenes), sugars (including glucuronic acids, glucosamines, and glucoses), polyols polyamines, phosphates, sulfates, sulfonates, phosphoramides, cyclopropanes, cyclobutanes, cyclopentanes, cyclohexanes, cyclopheptanes, furans, thiophenes, 2H-pyrroles, pyrroles, 2-pyrrolines, 3-pyrrolines, pyrrolidines, 1,3-dioxanes, oxazoles, oxazolines, thiazoles, imidazoles, 1-imidazolines, imidazolidines, pyrazoles, 2-pyrazolines, 3-pyrazolines, pyrazolidines, isoxazoles, isothiazoles, 1,2,3-oxadiazoles, 1,2,3-triazoles, 1,3,4-thiadiazoles, 2H-pyrans, thiazolidines, 4H-pyrans, pyridines, piperidines, 1,2-dioxanes, 1,4-dioxanes, 1,2-morpholines, 1,3-morpholines, 1,4-morpholines, 1,2-dithianes, 1,3-dithianes, 1,4-dithianes, 1,2-thiomorpholines, 1,3-thiomorpholines, 1,4-thiomorpholines, pyridazine, pyrimidines, pyrazines, 1,2-piperazines, 1,3-piperazines, 1,4-piperazines, 1,3,5-triazines, tetrazoles, 1,3,5-trithianes, 1,2,3,4-tetrahydro-1,3-diazines, indolizines, indoles, isoindoles, 3H-indoles, indolines, benzo[b]furans, benzo[b]thiophenes, 1H-indazoles, benzimidazoles, benzthiozoles, benzthioxoles, purines, 4H-quinolizines, quinolines, isoquinolines, cinnolines, phthalazines, quinazolines, quinoxalines, 1,8-naphthyridines, pteridines, quinuclidines, carbazoles, acridines, phenazines, phenothiazines, phenoxazines, indenes, naphthalenes, azulenes, fluorenes, anthracenes, norboranes, adamantanes, b-carbolines, perimidines, furazans, phenanthridines, phenanthrolines, phenarsazines, chromans, and isochromans.

14. The composition of claim 1, wherein the molecule comprises:

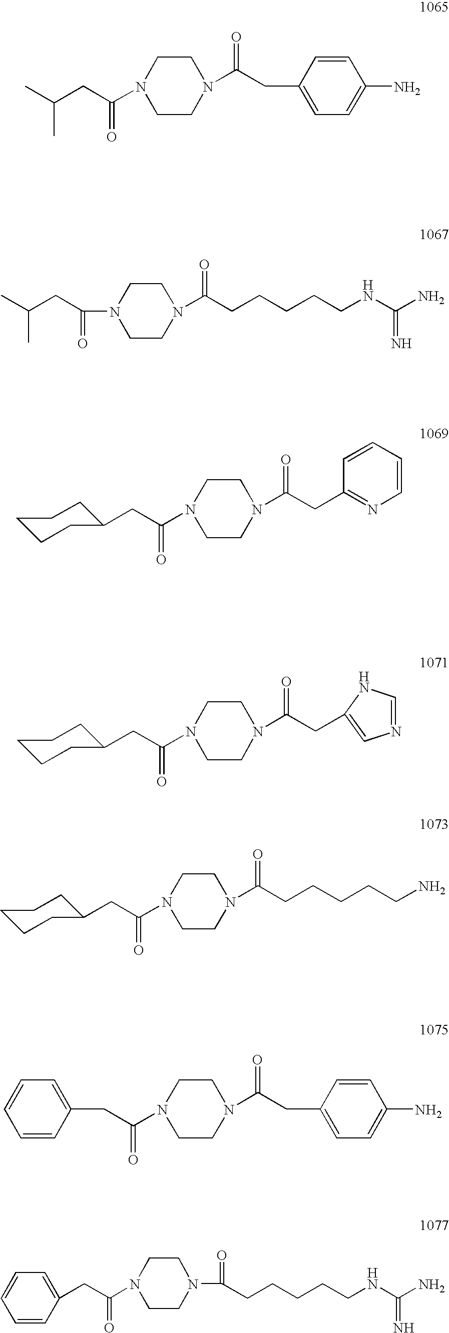

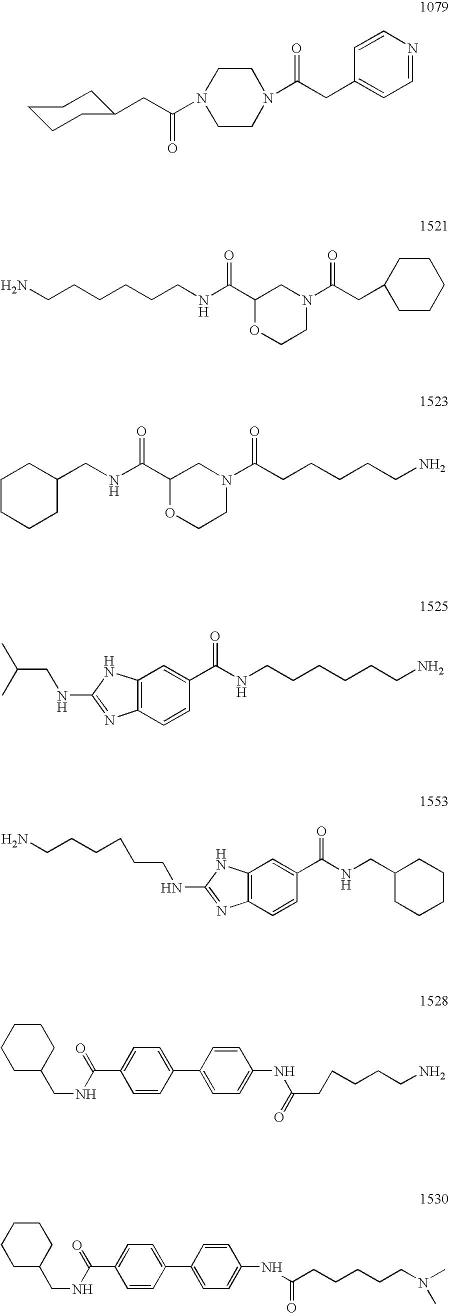

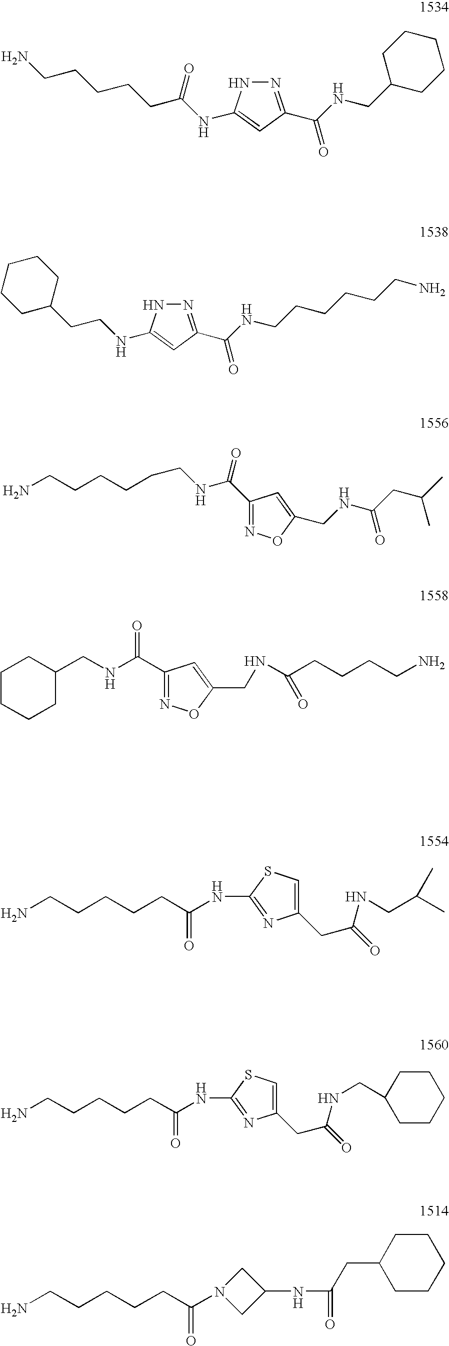

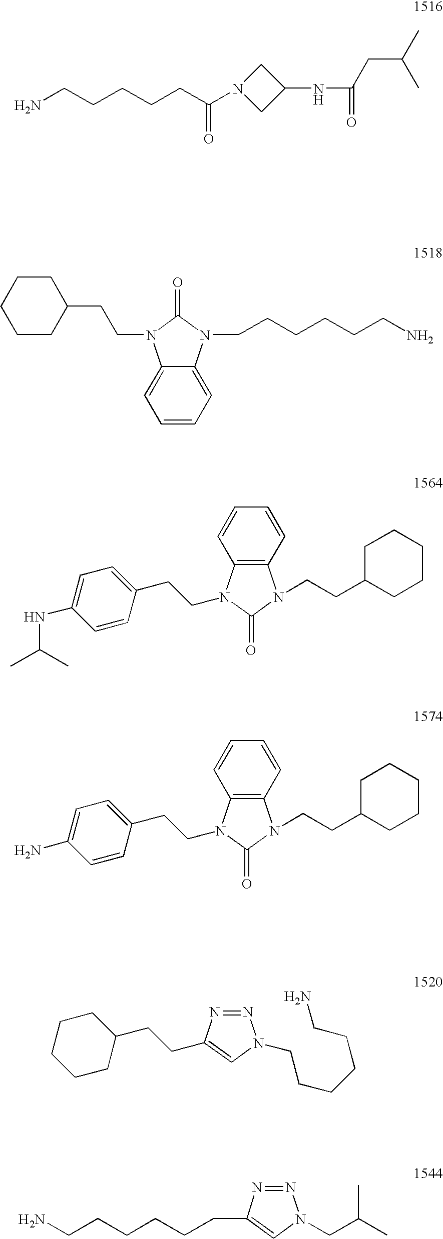

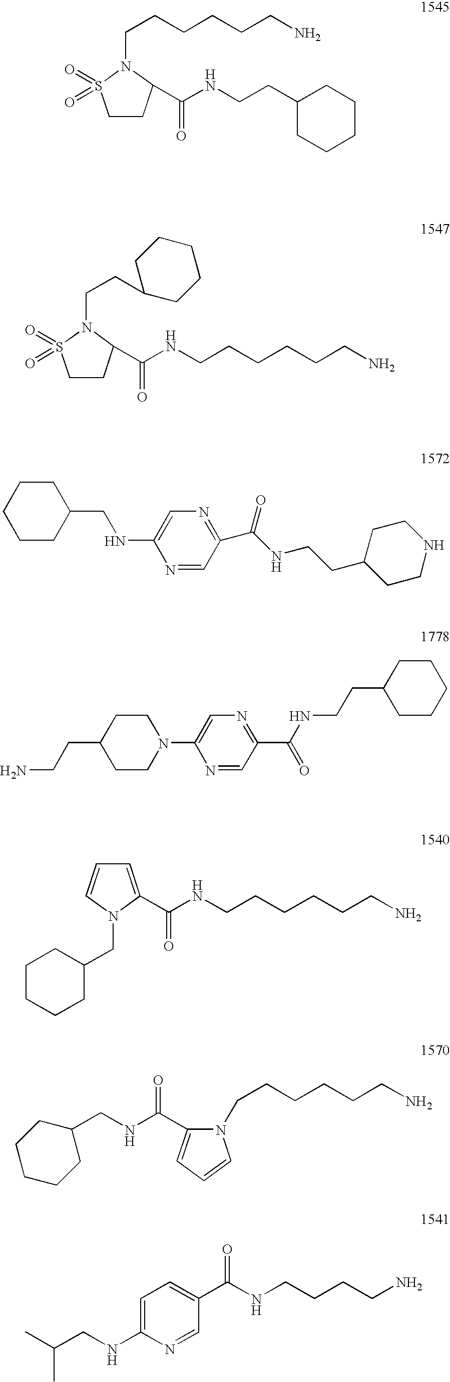

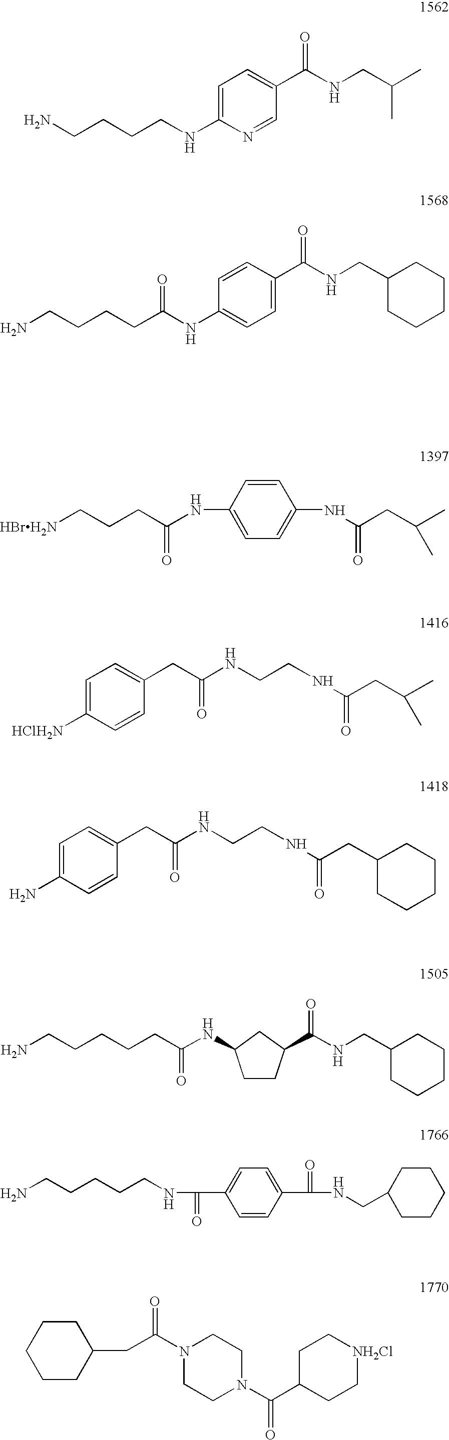

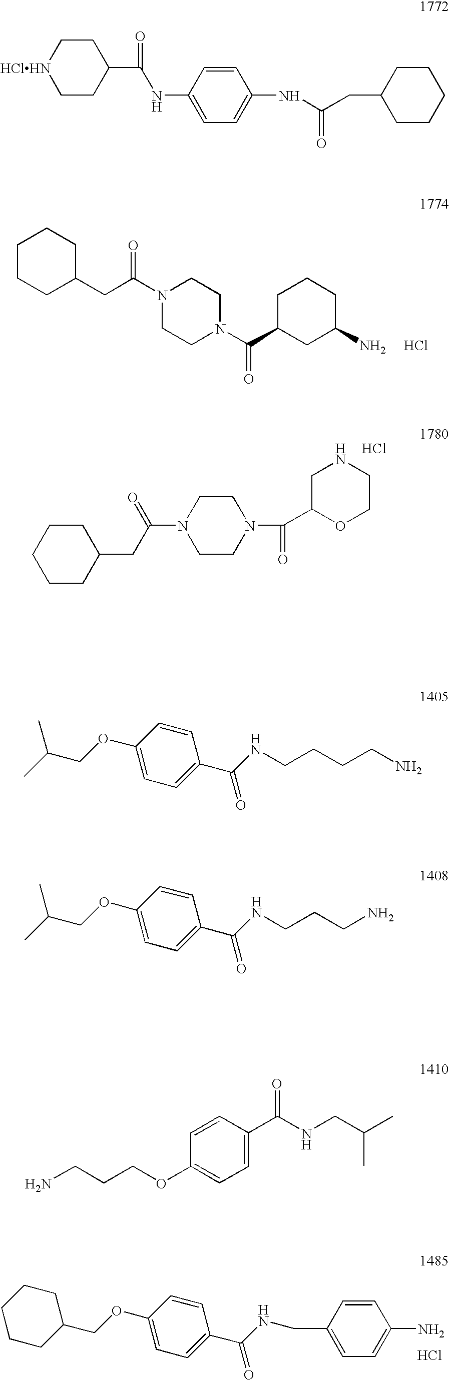

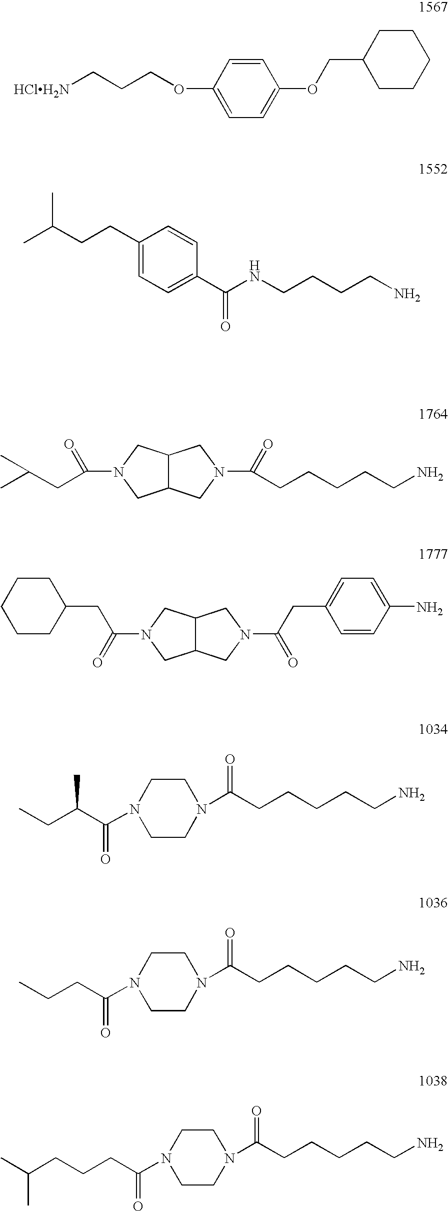

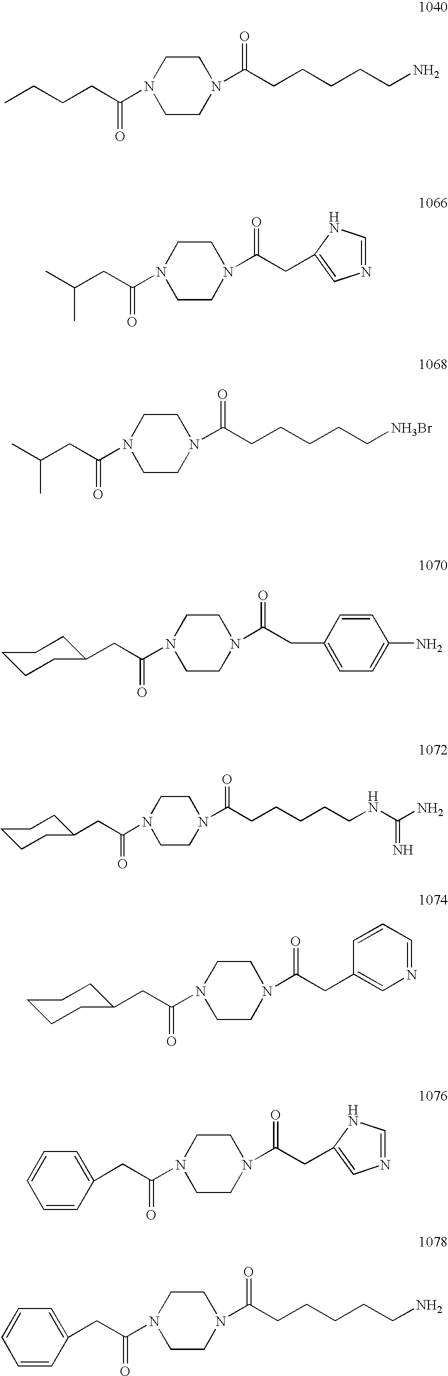

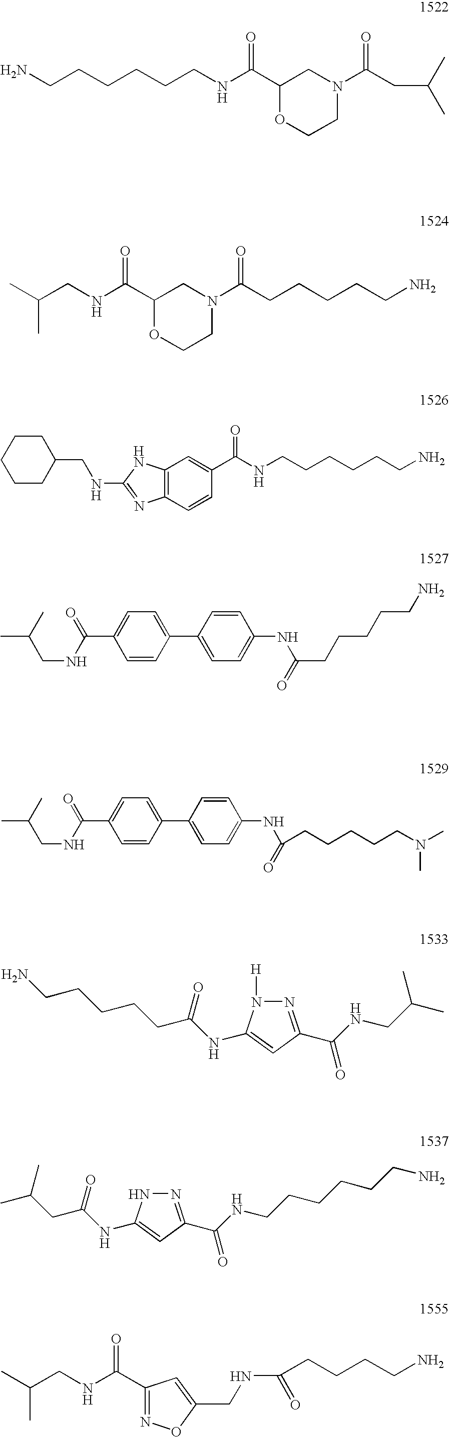

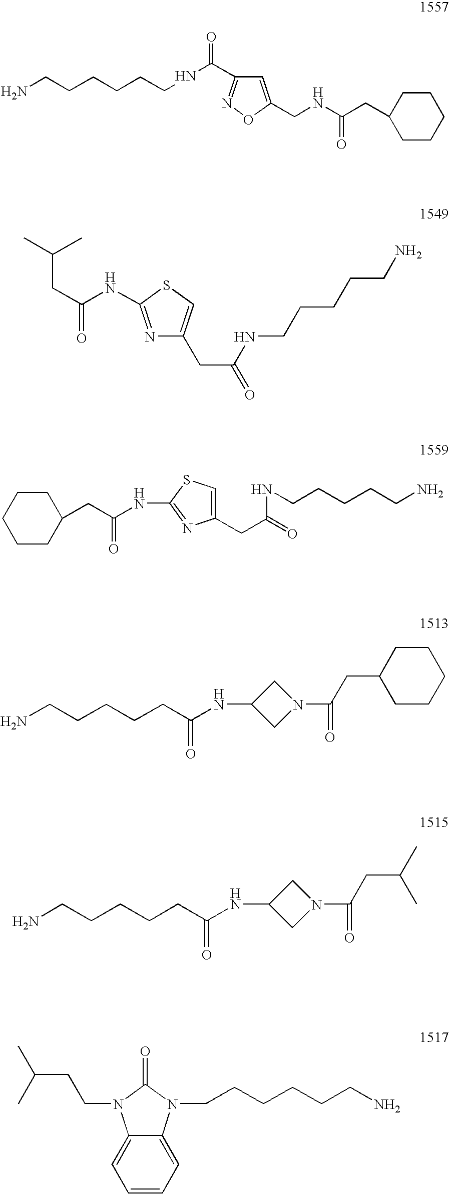

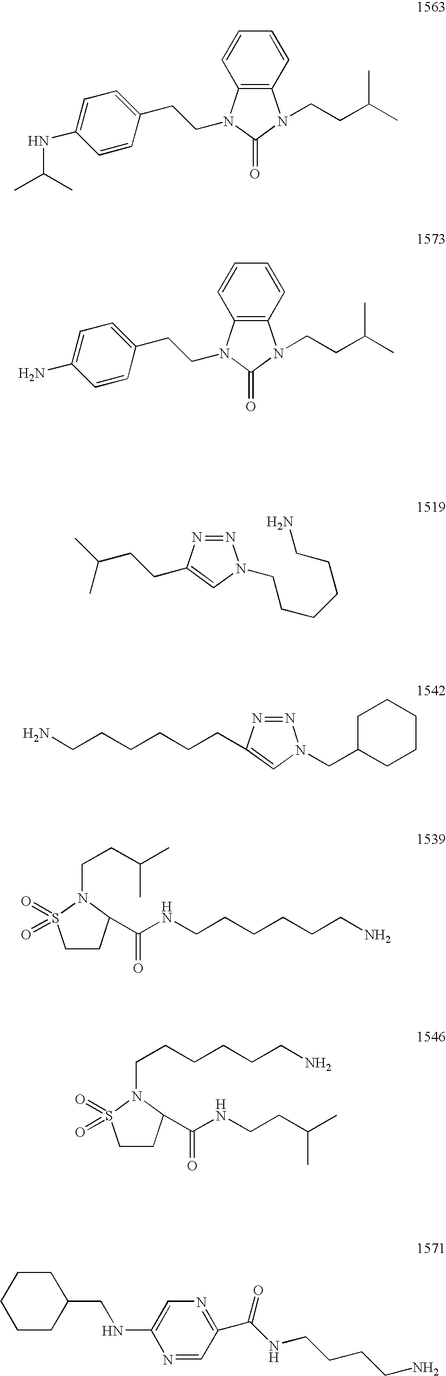

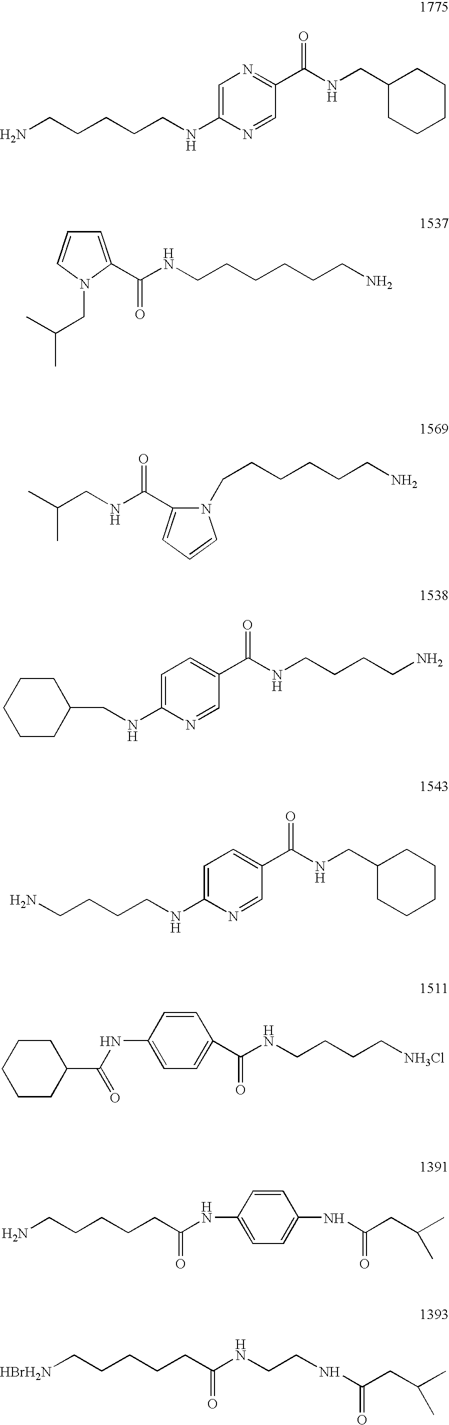

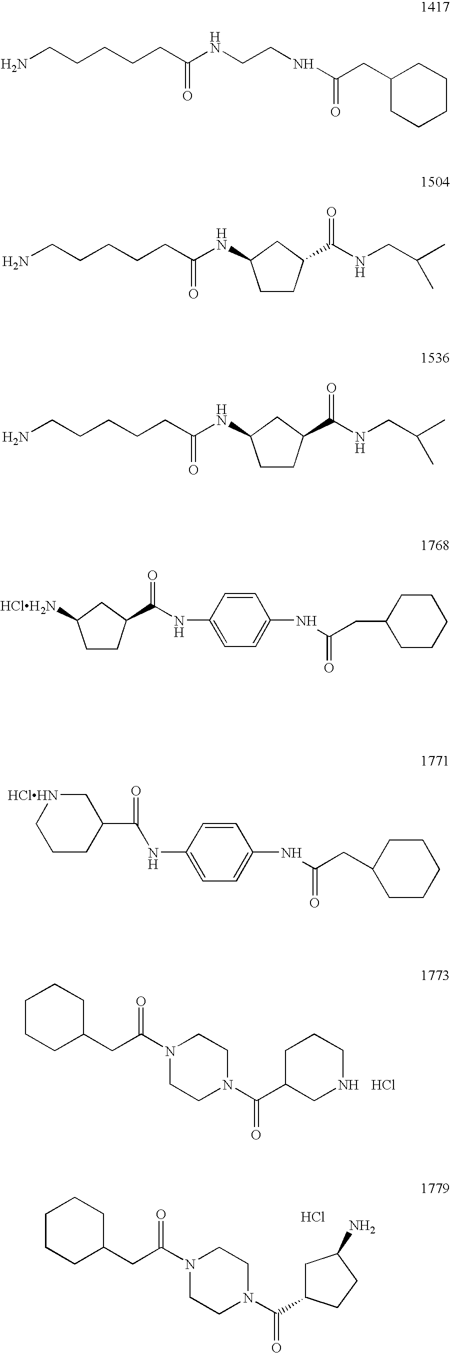

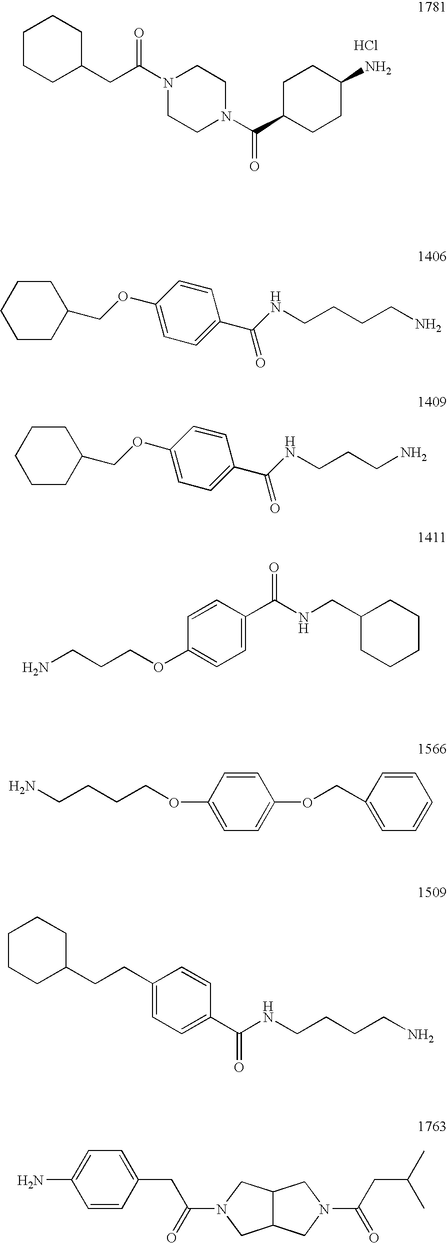

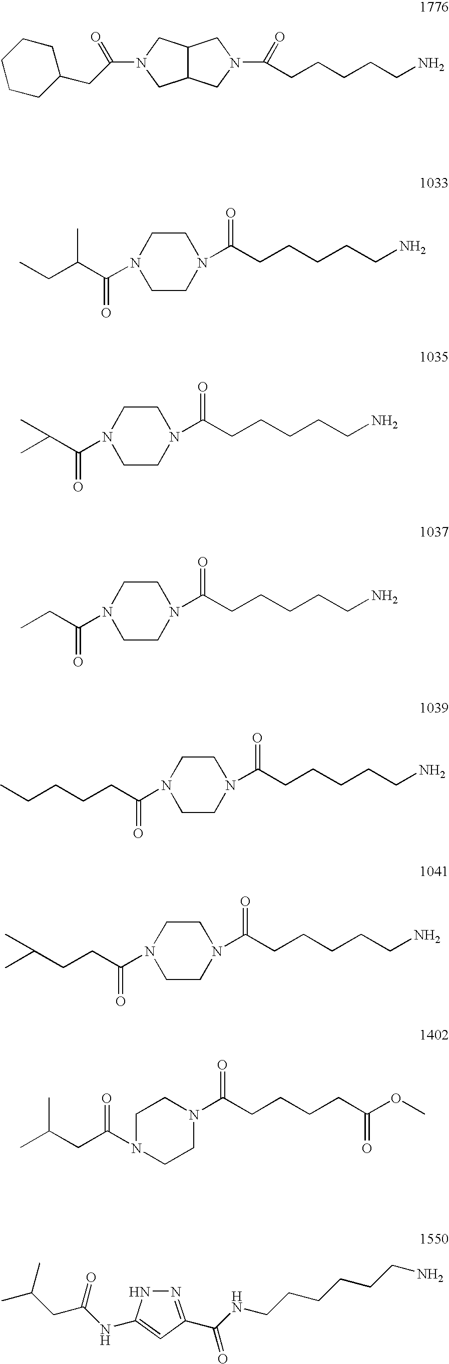

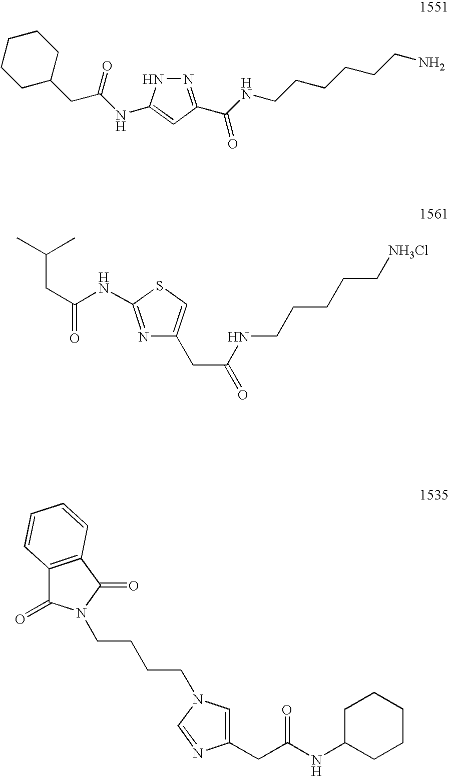

15. The composition of claim 1, further comprising a pharmaceutically acceptable carrier.

16. The composition of claim 1, wherein the composition is in the form of a solid, a liquid, an aerosol, a pill, a cream, an implantable dosage unit or an implantable dosage unit in a biodegradable polymer.

17. The composition of claim 1, wherein the composition comprises ENMD-1068.

18. A method for treating a human or animal having undesirable cellular proliferation, cancer or inflammation comprising administering to the human or animal a composition comprising a molecule;
wherein the molecule comprises a first component, a linker and a second component;
wherein the first component comprises a basic portion, a polar portion or a hydrogen-bonding portion;
wherein the second component comprises a hydrophobic moiety.

19. The method of claim 18, wherein the first component comprises an alcohol, amine, acid, guanine, ester or amide functional groups.

20. The method of claim 19, wherein the first component further comprises linear or branched, saturated or unsaturated carbocyclic rings.

21. The method of claim 19, wherein the first component further comprises linear or branched, saturated or unsaturated heterocyclic rings.

22. The method of claim 19, wherein the first component comprises a chemical moiety that structurally, spatially, chemically, or electronically mimics lysine.

23. The method of claim 18, wherein the second component comprises a substituted or unsubstituted straight or branched aliphatic.

24. The method of claim 18, wherein the second component further comprises saturated carbocyclic rings, unsaturated carbocyclic rings, saturated heteroatom-containing rings or unsaturated heteroatom-containing rings.

25. The method of claim 18, wherein the second component comprises a chemical moiety that stereochemically mimics leucine.

26. The method of claim 18, wherein the linker comprises a chemical moiety which structurally, spatially, chemically, or electronically mimics the spacing provided by the Ile and Gly residues in LIGK.

27. The method of claim 18, wherein the linker further comprises saturated aromatic ring systems, unsaturated aromatic ring systems, aromatic ring systems, linear unsaturated hydrocarbon chains, branched unsaturated hydrocarbon chains, linear saturated hydrocarbon chains, branched saturated hydrocarbon chains, sugars, nucleotides or nucleosides, single ring unsaturated carbocycles, single ring saturated carbocycles, multiple ring unsaturated carbocycles, multiple ring saturated carbocycles, single ring unsaturated heterocycles, single ring saturated heterocycles, multiple ring unsaturated heterocycles, multiple ring saturated heterocycles, heteroatoms, halides, nitrogen, oxygen, sulfur, silicon, selenium, or phosphorous.

28. The method of claim 18, wherein the linker is non-cyclic, wherein the terminal R groups are bound to any position on the linker, wherein the linkers have heteroatom-containing substituent groups, or wherein the linkers can have aliphatic groups other than simple linear or branched hydrocarbon chains.

29. The method of claim 28, wherein the heteroatom-containing substituent groups comprise imidazoles, aminos, arginyls, aminophenyls, pyridyls, thiols, alcohols, acids, esters, halides or amides.

30. The method of claim 18, wherein the linker comprises substituted or unsubstituted phenyls, benzyls, saturated or unsaturated branched or linear hydrocarbons (including alkanes, alkenes, or alkylenes), sugars (including glucuronic acids, glucosamines, and glucoses), polyols polyamines, phosphates, sulfates, sulfonates, phosphoramides, cyclopropanes, cyclobutanes, cyclopentanes, cyclohexanes, cyclopheptanes, furans, thiophenes, 2H-pyrroles, pyrroles, 2-pyrrolines, 3-pyrrolines, pyrrolidines, 1,3-dioxanes, oxazoles, oxazolines, thiazoles, imidazoles, 1-imidazolines, imidazolidines, pyrazoles, 2-pyrazolines, 3-pyrazolines, pyrazolidines, isoxazoles, isothiazoles, 1,2,3-oxadiazoles, 1,2,3-triazoles, 1,3,4-thiadiazoles, 2H-pyrans, thiazolidines, 4H-pyrans, pyridines, piperidines, 1,2-dioxanes, 1,4-dioxanes, 1,2-morpholines, 1,3-morpholines, 1,4-morpholines, 1,2-dithianes, 1,3-dithianes, 1,4-dithianes, 1,2-thiomorpholines, 1,3-thiomorpholines, 1,4-thiomorpholines, pyridazine, pyrimidines, pyrazines, 1,2-piperazines, 1,3-piperazines, 1,4-piperazines, 1,3,5-triazines, tetrazoles, 1,3,5-trithianes, 1,2,3,4-tetrahydro-1,3-diazines, indolizines, indoles, isoindoles, 3H-indoles, indolines, benzo[b]furans, benzo[b]thiophenes, 1H-indazoles, benzimidazoles, benzthiazoles, benzthioxoles, purines, 4H-quinolizines, quinolines, isoquinolines, cinnolines, phthalazines, quinazolines, quinoxalines, 1,8-naphthyridines, pteridines, quinuclidines, carbazoles, acridines, phenazines, phenothiazines, phenoxazines, indenes, naphthalenes, azulenes, fluorenes, anthracenes, norboranes, adamantanes, b-carbolines, perimidines, furazans, phenanthridines, phenanthrolines, phenarsazines, chromans, and isochromans.

31. The method of claim 18, wherein the composition comprises:

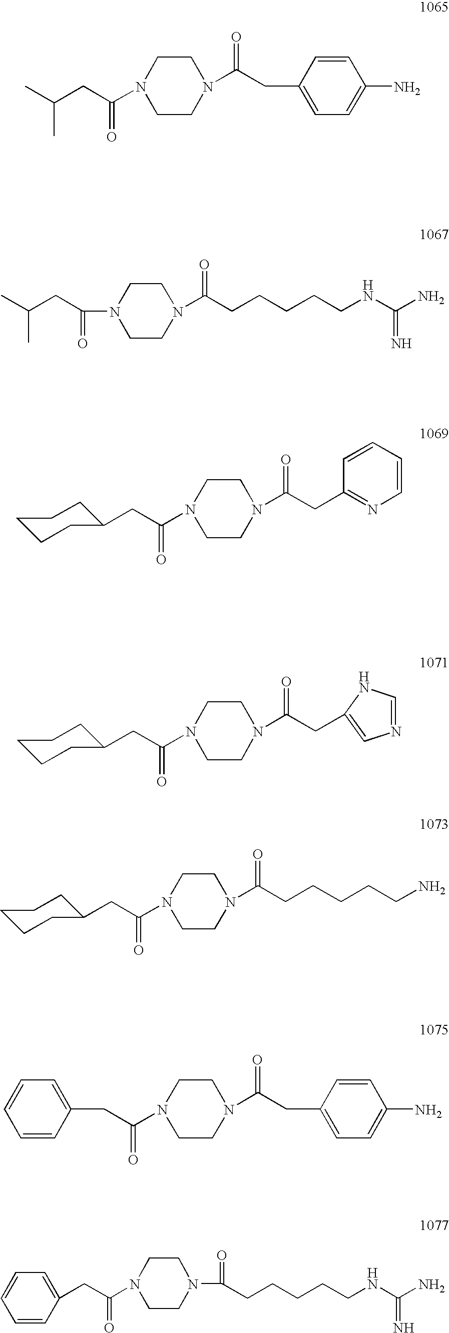

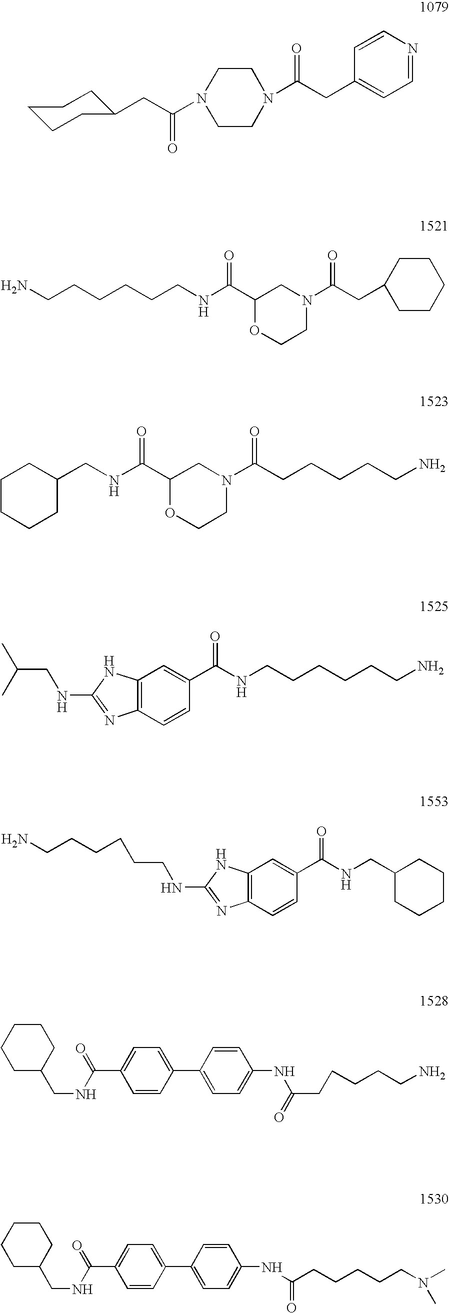

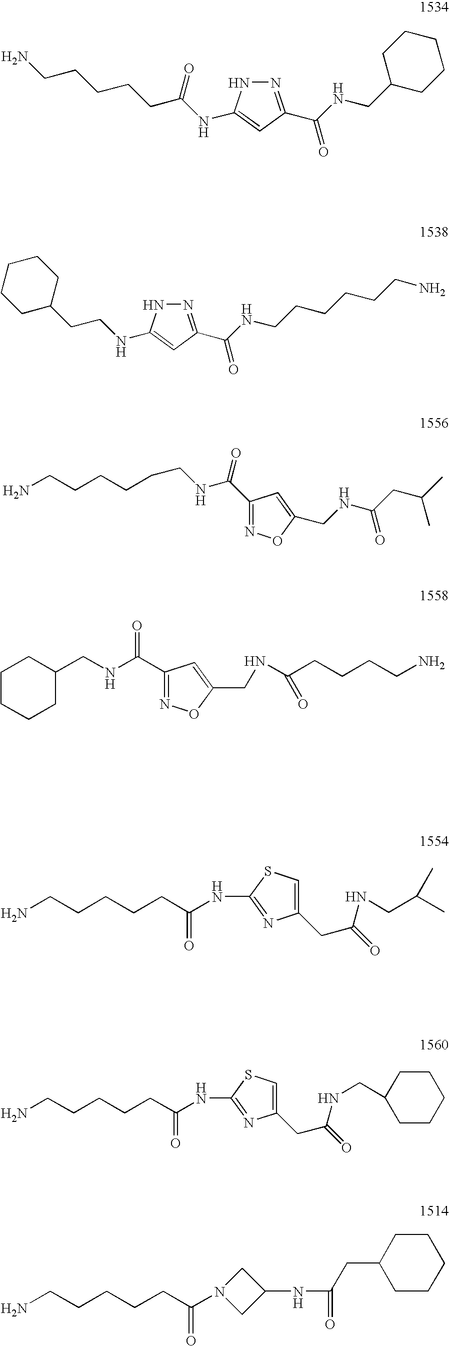

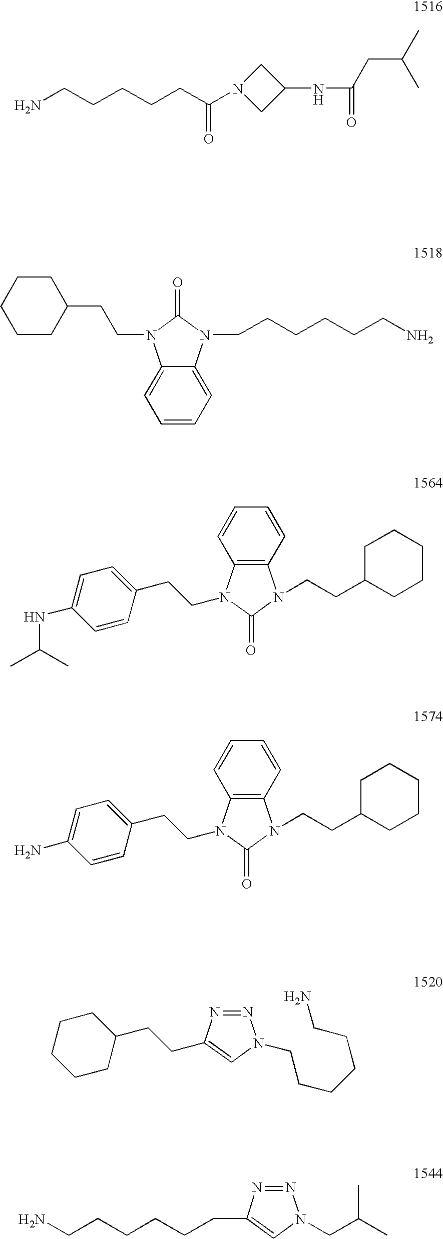

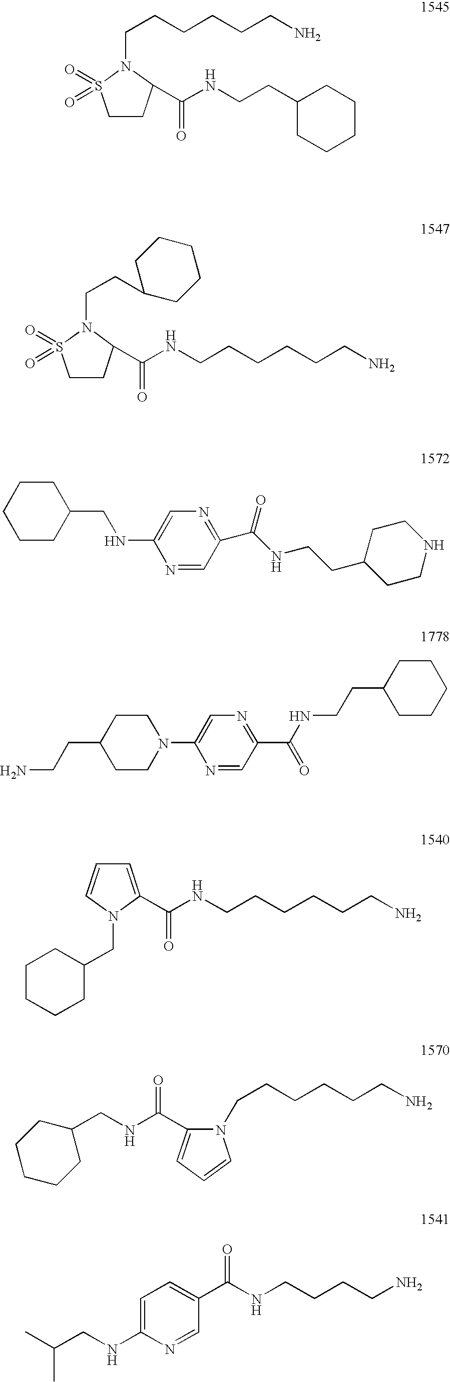

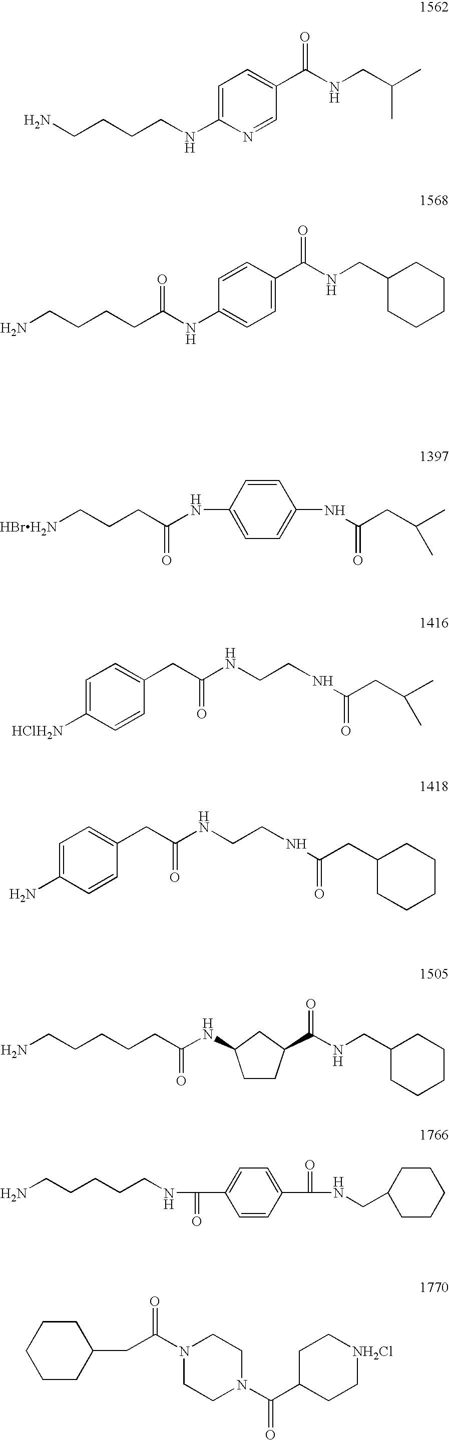

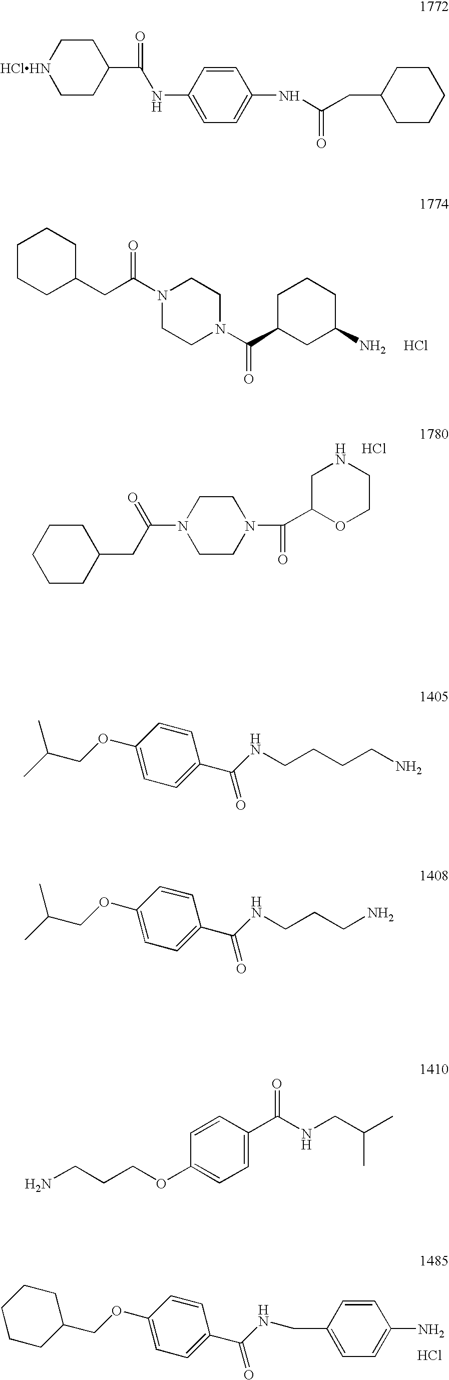

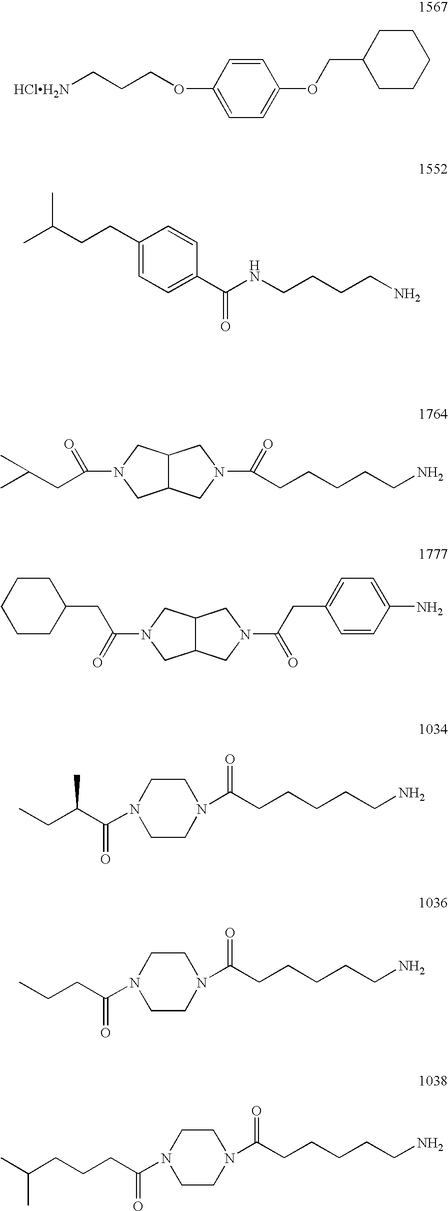

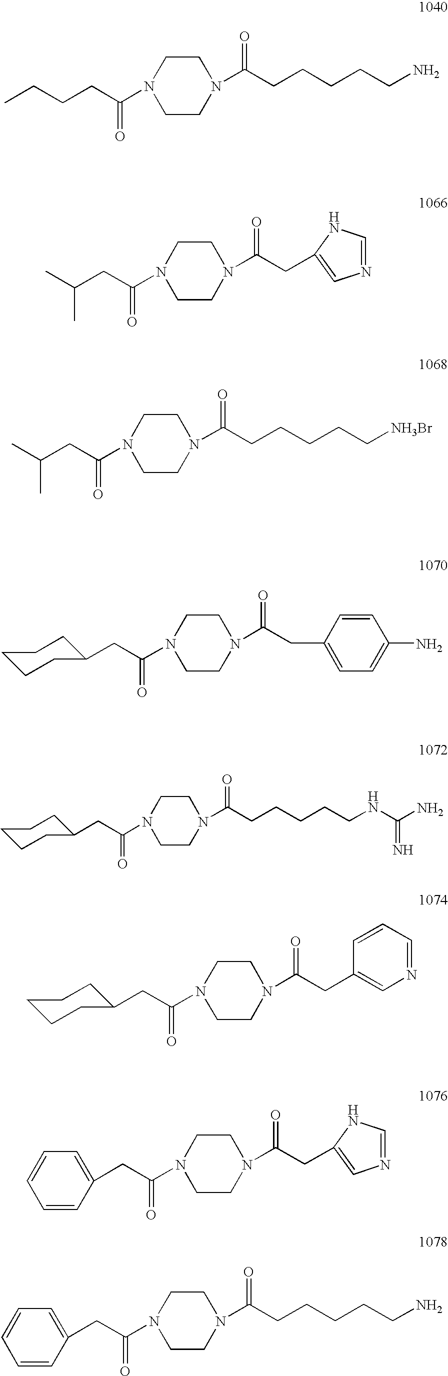

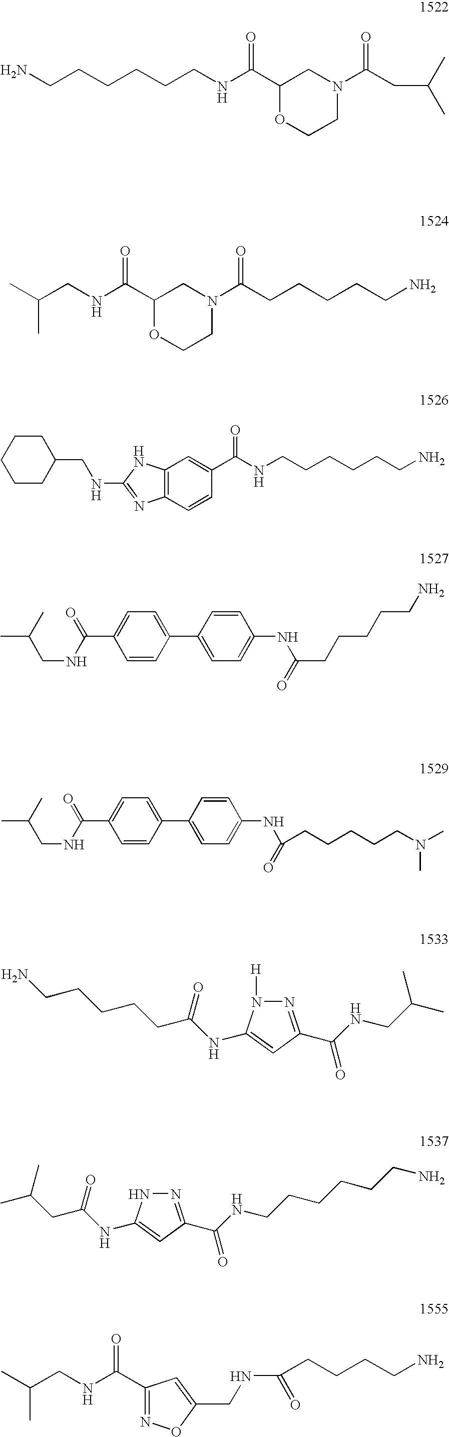

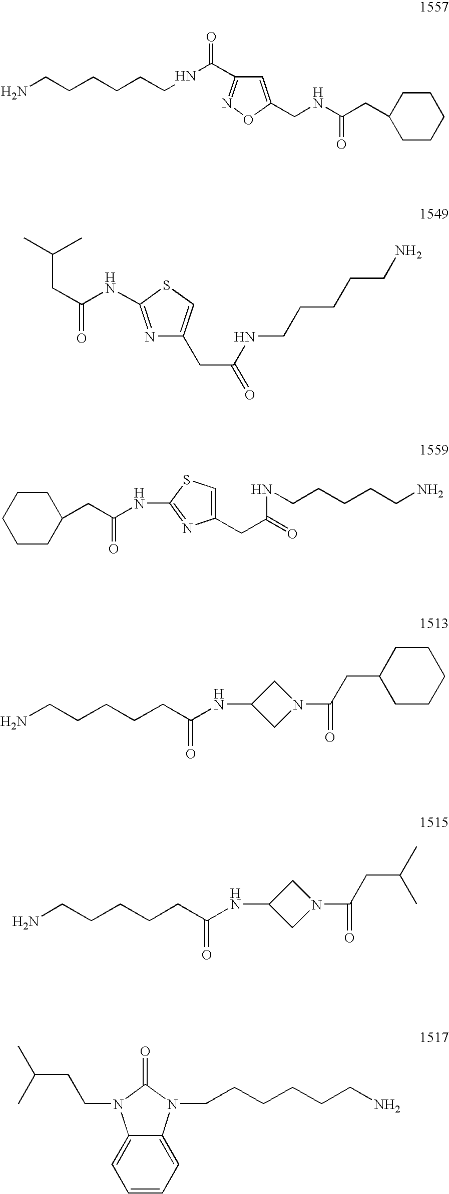

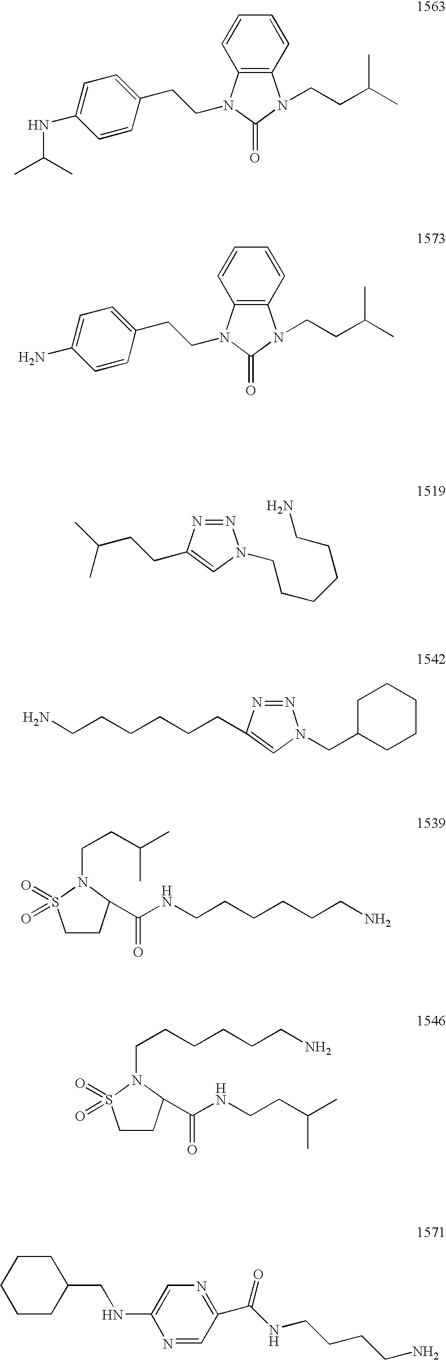

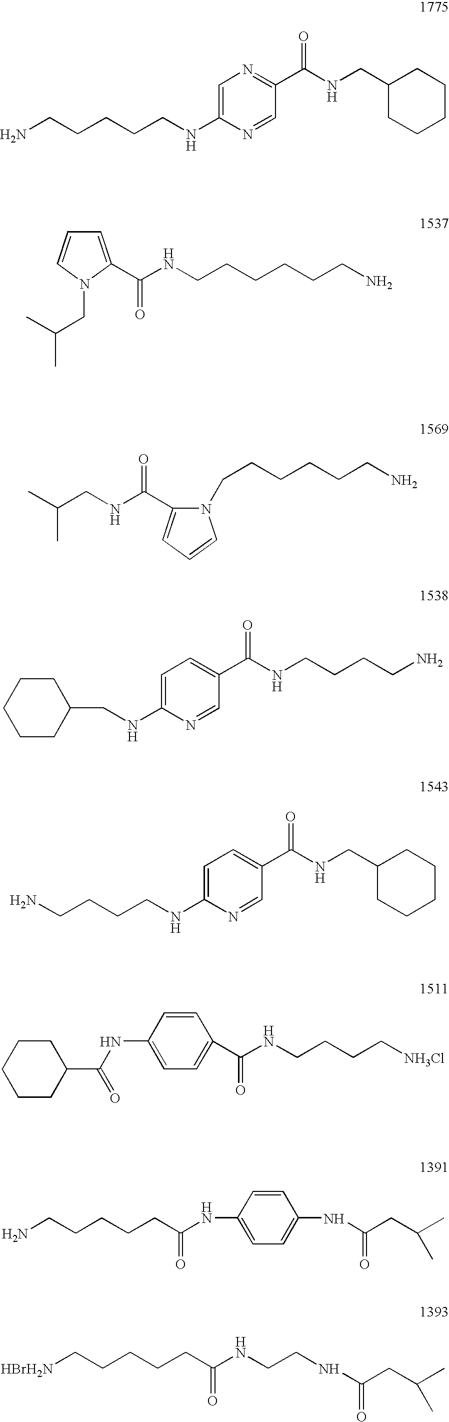

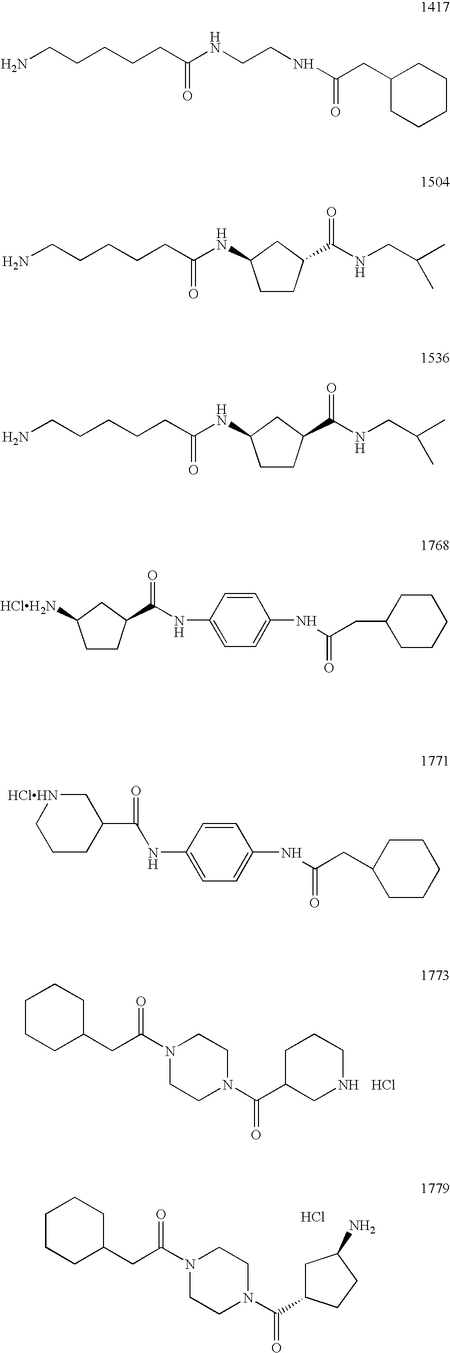

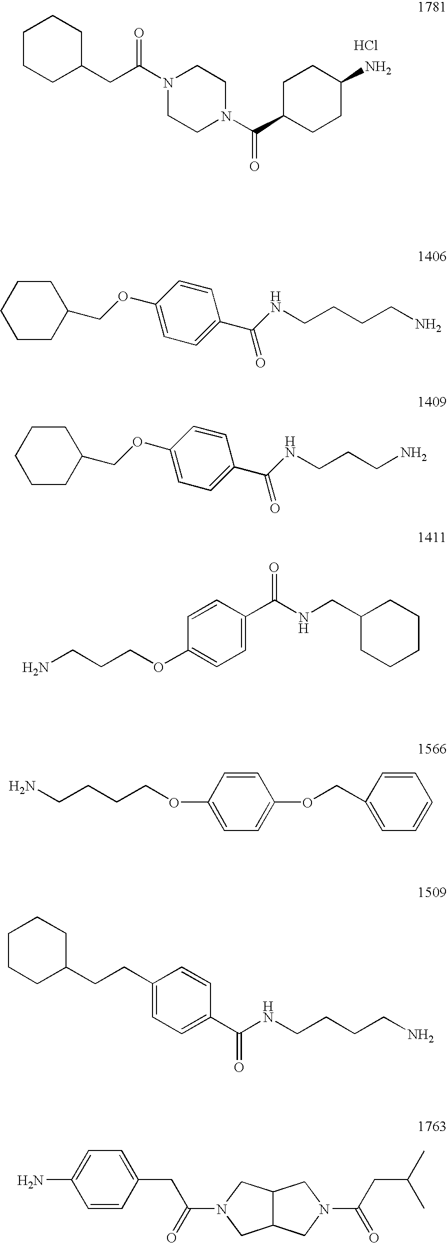

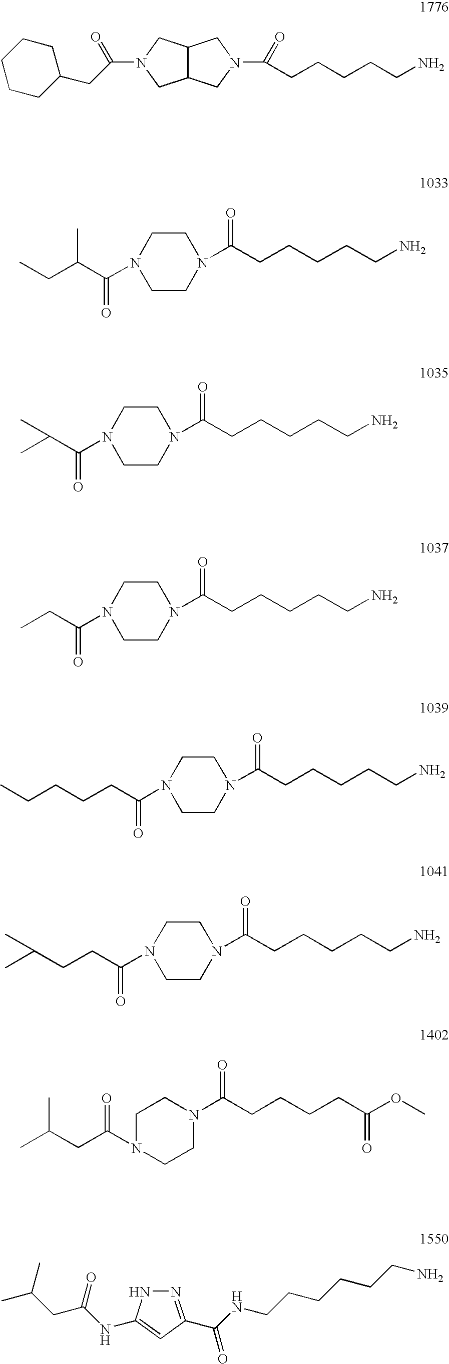

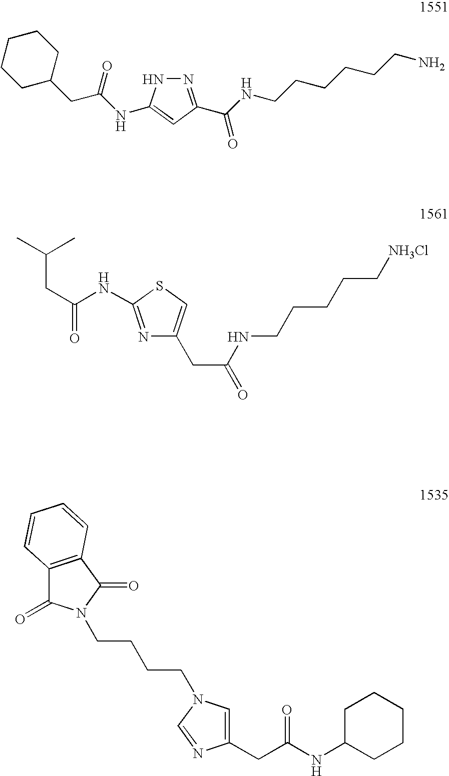

32. The method of claim 23, wherein the condition, disease or disorder comprises abnormal growth by endothelial cells, acne rosacea, acoustic neuroma, adhesions, angiofibroma, arteriovenous malformations, artery occlusion, arthritis, asthma, atherosclerosis, capillary proliferation within plaques, atherosclerotic plaques, atopic keratitis, bacterial ulcers, bartonelosis, Behcet's disease, benign tumors (such as, neurofibromas, trachomas, pyogenic granulomas), benign, premalignant and malignant vulvar lesions, Best's disease, bladder cancers, block implantation of a blastula, block menstruation (induce amenorrhea), block ovulation, blood-borne tumors (including leukemias, and neoplastic diseases of the bone marrow), bone marrow abnormalities including any of various acute or chronic neoplastic diseases of the bone marrow in which unrestrained proliferation of white blood cells occurs including multiple myeloma, bone growth and repair, breast cancer, burns, hypertrophy following cancer (including solid tumors: rhabdomyosarcomas, retinoblastoma, Ewing's sarcoma, neuroblastoma, osteosarcoma, blood-borne tumors, leukemias, neoplastic diseases of the bone marrow, multiple myeloma diseases and hemangiomas), carotid obstructive disease, central nervous system malignancy, certain immune reactions (for example immune disorders/reactions), cervical cancers, chemical burns, cholesteatoma especially of the middle ear, choroidal neovascularization, choroiditis, chronic or acute inflammation, chronically exercised muscle, cirrhotic liver, contact lens overwear, corneal diseases, corneal graft neovasularization, corneal graft rejection, corneal neovascularization diseases (including, but not limited to, epidemic keratoconjunctivitis, Vitamin A deficiency, contact lens overwear, atopic keratitis, superior limbic keratitis, and pterygium keratitis sicca), corpus luteum formation, Crohn's disease, delayed wound healing, diabetes, diabetic (proliferative) retinopathy, diseases caused by the abnormal proliferation of fibrovascular or fibrous tissue including all forms of prolific vitreoretinopathy, Eales disease, embryo development, empyema of the thorax, endometriosis, endometrium, epidemic keratoconjuctivitis, Ewing's sarcoma, excessive or abnormal stimulation of endothelial cells (such as atherosclerosis), eye-related diseases (including rubeosis (neovascularization of the angle), abnormal proliferation of fibrovascular or fibrous tissue, including all forms of prolific vitreoretinopathy), female reproductive system conditions (including neovascularization of ovarian follicles, corpus luteum, maternal decidua, repair of endometrial vessels, angiogenesis in embryonic implantation sites (ovarian hyperstimulation syndromes), embryonic development, folliculogenesis, luteogenesis, normal menstruating endometrium), fibrinolysis, fibroplasias (retrolental and excessive repair in wound healing), fibrosing alveolitis, fungal ulcers, gastrointestinal infections, peptic ulcer, ulcerative colitis, inflammed polyps, intestinal graft-vs-host reaction, neoplastic tumors, mastocytosis, intestinal ischemia, neovascular glaucoma, gout or gouty arthritis, graft versus host rejection (also chronic and acute rejection), granulation tissue of healing wounds, burn granulations, haemangiomatoses (systemic forms of hemangiomas), hand foot and mouth disease, hair growth, hemangioma, hemophiliac joints, hereditary diseases (including hereditary hemorrhagic telangiectasia), herpes simplex, herpes zoster, HHT (hereditary hemorrhagic telangiectasia), hypertrophic scars, hypertrophy following surgery, burns and injury, hyperviscosity syndromes, immune disorders, immune reactions, implantation of embryo (2-8 weeks), infections causing retinitis, infectious diseases caused by microorganisms, inflammation, inflammatory disorders, immune and non-immune inflammatory reactions, inflammed joints, Kaposi's sarcoma, leprosy, leukemias, lipid degeneration (lipid keratopathy), lipoma, lung cancer, lupus (lupus erythematosis, systemic lupus erythematosis), lyme disease, age-related macular degeneration (subretinal neovascularization), marginal keratolysis, melanoma, meningiomas, mesothelioma, metastasis of tumors, Mooren's ulcer, mycobacteria diseases, myeloma, multiple myeloma diseases, myopia, neoplasias, neoplastic diseases of the bone marrow, neovascular glaucoma, neurofibroma, neurofibromatosis, neurofibrosarcoma, non-union fractures, ocular angiogenic diseases (including diabetic retinopathy, retinopathy of prematurity, and retrolental fibroplasia, macular degeneration, corneal graft rejection, neovascular glaucoma), ocular histoplasmosis, ocular neovascular disease, ocular tumors, optic pits, oral cancers, Osler-Weber syndrome (Osler-Weber-Rendu disease), osteoarthritis, osteomyelitis, osteosarcoma, Paget's disease (osteitis deformans), parasitic diseases, pars planitis, pemphigoid, phlyctenulosis, polyarteritis, post-laser complications, proliferation of white blood cells (such as any of various acute or chronic neoplastic diseases of the bone marrow, in which unrestrained proliferation of white blood cells occurs), prolific vitreoretinopathy (PVR), prostate cancer, protozoan infections, pseudoxanthoma elasticum, psoriasis, pterygium (keratitis sicca), pulmonary fibrosis, radial keratotomy, chronic and acute rejection, retinal detachment, retinitis, retinoblastoma, retinopathy of prematurity, retrolental fibroplasia, rhabdomyosarcomas, rheumatoid arthritis, rheumatoid synovial hypertrophy (arthritis), rosacea, rubeosis, sarcoidosis, scleritis, scleroderma, sicca (including pterygium (keratitis sicca) and Sjogren's (sicca) syndrome), sickle cell anemia, Sjogren's (sicca) syndrome, skin disease (including melanoma), pyogenic granulomas, psoriasis, hemangioma, skin warts, and HPV type 2 (human papillomavirus)), solid tumors, Stargard's disease, Stevens-Johnson's disease, superior limbic keratitis (superior limbic keratoconjuctivitis, SLK), hypertrophic scars, wound granulation and vascular adhesions, syphilis, Terrien's marginal degeneration, toxoplasmosis, trachoma, trauma, tuberculosis, tumors, tumor associated angiogenesis, tumor growth, ulcerative colitis, ulcers (including fungal, Mooren's, peptic and bacterial), undesired angiogenesis in normal processes (including wound healing, female reproductive functions, bone repair, hair growth, chronic uveitis, and vascular malfunction), vascular tumors, vein occlusion, vitamin A deficiency, chronic vitritis, Wegener's sarcoidosis, white blood cells diseases (including any acute or chronic neoplastic diseases of the bone marrow in which unrestrained proliferation of white blood cells occurs), wound healing and inappropriate wound healing, delayed wound healing (for instance in angiofibroma, arteriovenous malformations, arthritis, atherosclerotic plaques, corneal graft neovascularization, diabetic retinopathy, hemangioma, hemophilic joints, hypertrophic scars, neovascular glaucoma, non-union fractures, Osler-Weber syndrome, psoriasis, pyogenic granuloma, retrolental fibroplasias, scleroderma, solid tumors, trachoma, corpus luteum formations, chronically exercised muscle, psoriasis, diabetic retinopathy, tumor vascularization, rheumatoid arthritis, psoriasis, solid tumors, and chronic inflammatory diseases, inflamed joints, rheumatoid synovial hypertrophy (arthritis), atherosclerosis, proliferative (diabetic) retinopathy, tumor growth, metastasis, oral cancers, cervical cancers, bladder and breast cancers, melanomas, pyogenic granulomas, tumors, diabetic retinopathy, psoriasis, rheumatoid arthritis, haemangiomatoses, Kaposi's sarcoma, adhesions, acute and/or chronic inflammation and inflammatory reactions, and chronic and acute rejection), asthma, bronchogenic carcinoma, sarcoidosis, ankylosing spondylosis, chronic obstructive pulmonary disease, thyroiditis (including subacute, acute and chronic thyroiditis, granulomatous (or DeQuervain's thyroiditis) lymphocytic thyroiditis (Hashimoto's thyroiditis), invasive fibrous (Riedel's) thyroiditis, pyogenic or suppurative thyroiditis), dermatitis (including psoriasis, eczema, dermatitis, seborrheic dermatitis, contact dermatitis, atopic dermatitis, nummular dermatitis, chronic dermatitis, lichen simplex chronicus, stasis dermatitis, generalized exfoliative dermatitis and Behçt's Syndrome), adenomatous polyposis coli, Alagille syndrome, appendicitis, Barrett esophagus, biliary atresia, biliary tract diseases, Caroli disease, celiac disease, cholangitis, cholecystitis, cholelithiasis, ulcerative colitis, Crohn's disease, digestive system diseases, duodenal ulcer, dysentery, pseudomembranous enterocolitis, esophageal achalasia, esophageal atresia, esophagitis, fatty liver, gastritis, hypertrophic gastritis, gastroenteritis, gastroesophageal reflux, gastroparesis, hepatitis, chronic hepatitis, Hirschsprung disease, inflammatory bowel diseases, intestinal neoplasms, intestinal neuronal dysplasia, liver cirrhosis, Meckel diverticulum, pancreatic diseases (including pancreatic insufficiency, pancreatic neoplasms, and pancreatitis), peptic ulcer, Peutz-Jeghers syndrome, proctitis, Whipple disease, Zollinger-Ellison syndrome, multiple sclerosis, neuritis, Alzheimer's disease and other neurological diseases, bronchiolitis obliterans organising pneumonia, bronchiectasis, pulmonary fibrosis, chronic obstructive pulmonary syndrome, systemic sclerosis, pleural inflammation, seronegative spondylarthropathies, septic arthritis, prolonged pulmonary eosinophilia, simple pulmonary eosinophilia, Löffler's syndrome, pulmonary eosinophilia with asthma, polyarteritis nodosa, chronic eosinophilic pneumonia, acute eosinophilic pneumonia, idiopathic hypereosinophilic syndrome, allergic bronchopulmonary aspergillosis, bronchocentric granulomatosis, allergic angiitis and granulomatosis (Churg-Strauss Syndrome), idiopathic pulmonary fibrosis, Langerhan's cell granulomatosis (Eosinophilic Granuloma), chronic bronchitis, emphysema, interstitial pneumonia, cutaneous mastocytoma, urticaria pigmentosa, telangiectasia macularis eruptiva perstans (TMEP), systemic mast cell disease, mast cell leukemia, eosinophilic fasciitis, eosinophilic gastroenteritis, eosinophilia myalgia syndrome, systemic mastocytosis, mastocytosis, reactive mastocytosis, neuritis, vestibular neuritis, optic neuritis, lupus nephritis, nephritis, and Parkinson's diseases.

33. The method of claim 25, wherein the composition is administered orally, topically, implanted locally, implanted for systematic release, implanted for sustained release, implanted in a biodegradable particle, subcutaneously, subcutaneously, intravenously, intra-arterially, intraocularly, transdermally, or transbuccally.

34. A composition comprising a molecule containing a linker, said molecule having the general structure of: 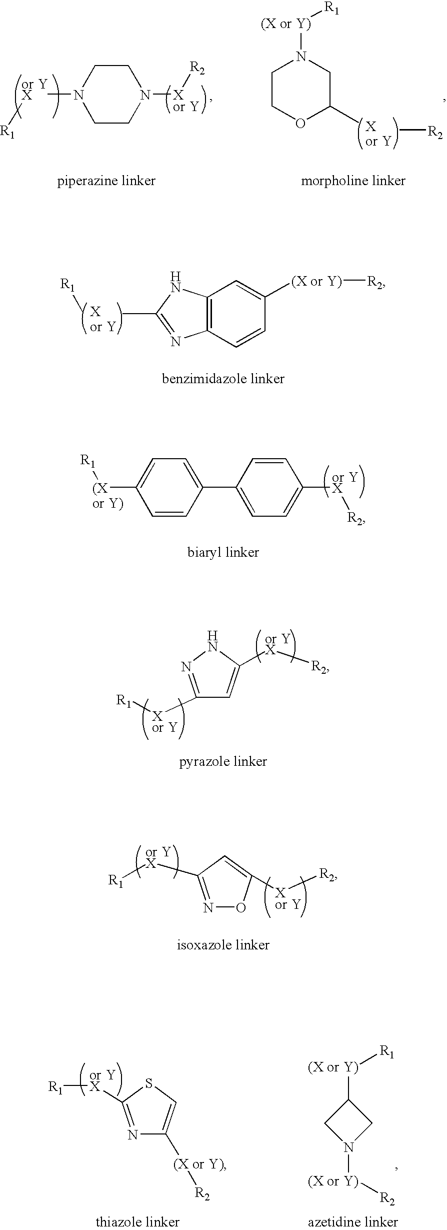 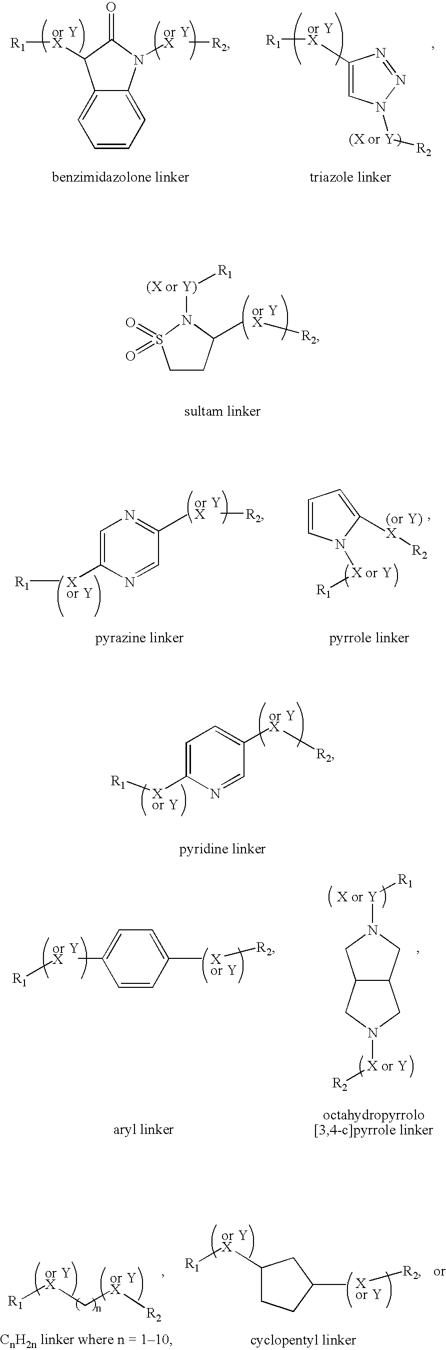 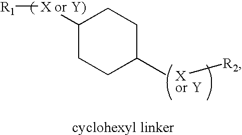
wherein the coupling group X or Y to a C of the linker can be independently —CₙH₂ₙ (n=1 to 4)-, —O—, —NH—, —CH₂NH—, —CH═CH—, —CH₂CH═CH—, —C≡C—, —CH₂C≡C—, —C(═O)—, —CH₂—C(═O)—, —C(═O)O—, —CH₂C(═O)O, —O—C(═O), —C(═O)NH—, —CH₂C(═O)NH—, —NH—C(═O)—, —CH2NH—C(═O)—, —NH—C(═O)—NH—, —CH₂NH—C(O)—NH—, —O—C(═O)—NH—, —CH₂O—C(═O)—NH—, —CH2NH—C(═O)—O—, or —NH—C(═O)—O—;
wherein coupling group X or Y to an N of the linker can be independently —CₙH₂ₙ (n—1 to 4), —CH═CH—, —CH₂CH═CH—, —C≡C—, —CH₂—C≡C—, —C(═O)—, —CH₂—C(═O)—, —C(═O)O—, —CH₂C(═O)O— —C(═O)NH—, or —CH₂C(═O)NH—;
wherein R₁ or R₂ can be independently either a hydrophobic or a hydrophilic substituent, wherein the hydrophobic substituent can be straight or branched aliphatic chains of 1-10 carbons, linear, branched or cyclic, and may be saturated or unsaturated or aromatic; and
wherein the hydrophilic substituent can be −2-morpholine, -3-morpholine, -4-morpholine, -2-thiomorpholine, -3-thiomorpholine, -4-thiomorpholine, -2-pyridine, -3-pyridine, -4-pyridine, -2-cyclohexylamine, -3-cyclohexylamine, -4-cyclohexylamine, -2-cyclopentylamine, -3-cyclopentylamine, -2-cyclobutylamine, -3-cyclobutylamine, -2-piperidine, -3-piperidine, -4-piperidine, -2-piperazine, -3-piperazine, -2-pyrrolidine, -3-pyrrolidine, -2-pyrrole, -3-pyrrole, -3-pyrazole, -4-pyrazole, -5-pyrazole, -2-imidazole, -4-imidazole, -5-imidazole, -2-azetidine, -3-azetidine,
—CH₂ₙ—NR₃R₄ where n=2-8 and R₃ and R₄ are independently hydrogen, methyl, ethyl, propyl or iso-propyl,
—CₙH₂ₙ—NHC(═NH)NH₂ where n=2-8,
—C₆H₄—NR₃R₄ where R₃ and R₄ are independently hydrogen, methyl, ethyl, propyl or iso-propyl,
—CH₂, —OH where n=2-8,
—CH₂, —COOR₃ where n=2-8 and R₃ is independently hydrogen, methyl, ethyl, propyl or iso-propyl, or
—CₙH₂ₙ—CONR₃R₄ where n=2-8 and R₃ and R₄ are independently hydrogen, methyl, ethyl, propyl, iso-propyl.
